(19) United States
(12) Patent Application Publication
(10) Pub. No.: US 2019/0385718 A1
(43) Pub. Date: Dec. 19, 2019

(54) METHODS FOR LOWERING TRIGLYCERIDE LEVELS WITH A CONCENTRATED FISH OIL-BASED PHARMACEUTICAL COMPOSITION

(71) Applicant: AstraZeneca UK Limited, Cambridge (GB)

(72) Inventors: Richard L. Skelly, Flourtown, PA (US); Judy Firor, Landenberg, PA (US)

(21) Appl. No.: 16/440,708

(22) Filed: Jun. 13, 2019

Related U.S. Application Data
(60) Provisional application No. 62/685,214, filed on Jun. 14, 2018.

(51) Int. Cl. G16H 20/10; G16H 10/20; G06F 16/335; A61K 35/63; A61K 35/60; A61K 31/202

(52) U.S. Cl. CPC G16H 20/10; G16H 10/20; G06F 16/335; G06Q 30/0635; A61K 35/60; A61K 31/202; A61K 35/63

(57) ABSTRACT

A method is provided for lowering triglyceride levels in a subject in need thereof by administering a concentrated fish oil-based pharmaceutical composition to a subject qualified for over-the-counter access to the concentrated fish oil-based pharmaceutical composition. In some embodiments, the concentrated fish oil-based pharmaceutical composition includes an omega-3-carboxylic acid pharmaceutical composition. In some embodiments, the concentrated fish oil-based pharmaceutical composition includes an omega-3 acid ethyl ester pharmaceutical composition, an ethyl eicosapentaenoic acid pharmaceutical composition, or a krill oil pharmaceutical composition.

### CROSS REFERENCE TO RELATED APPLICATIONS

This application claims priority to U.S. Provisional Patent Application Ser. No. 62/685,214, filed Jun. 14, 2018, which is hereby incorporated by reference in its entirety.

### TECHNICAL FIELD

The present disclosure relates generally to methods for lowering triglyceride levels, e.g., thereby treating or preventing hypertriglyceridemia and/or heart disease, by administering an over-the-counter concentrated fish oil-based pharmaceutical composition to a subject in need thereof, who has been qualified for over-the-counter access to the composition.

### BACKGROUND

Cardiovascular disease is the leading global cause of death, claiming more lives than all forms of cancer combined. The number of cardiovascular deaths is expected to increase to approximately 24 million annually by 2030. The direct and indirect annual costs total more than $316 billion dollars. This exceeds the entire GDP of all the world's countries except the top 30 countries. Hyperlipidemia, e.g., high cholesterol, is a major risk factor for cardiovascular disease. Hyperlipidemia may be caused by either, or both, high cholesterol levels (e.g., hypercholesterolemia) or high triglyceride levels (hypertriglyceridemia) in the blood.

Fortunately, hypertriglyceridemia can be managed, for example, using concentrated fish oils, which are well established prescription pharmaceuticals used to lower triglycerides, thereby treating and/or preventing heart disease. For instance, the efficacy of very high purity omega-3-fatty acids, which was first approved in the U.S. for the treatment of hypertriglyceridemia in 2004, to reduce triglyceride levels has been demonstrated in numerous clinical trials. Gutstein A S et al., Cardiovascular disease and omega-3s: Prescription products and fish oil dietary supplements are not the same., J Am Assoc Nurse Pract., 29(12):791-801 (2017). Although supplements containing fish oil are available, e.g., at health and nutrition stores, concerns about the quality, purity, safety, and variability of content in these supplements abound. Id; Fialkow J., Am J Cardiovasc Drugs, 16(4):229-39 (2016). In contrast, well regulated, highly-concentrated fish oil is only available through a prescription. Unfortunately, long-term trends demonstrate many people avoid prescription medications, including concentrated fish oils.

One approach to improving the public's accessibility to high purity concentrated fish oils is to make them available without a prescription, e.g., over the counter (“OTC”). There are a variety of public health benefits that accompany switching a pharmaceutical drug from prescription to OTC, including, generating wider availably to therapies, providing a greater number of therapeutic approaches, providing direct and rapid access to treatments, providing patients with an active role in their own health care, and allowing patients to become self-reliant in preventing and relieving minor symptoms or conditions. World Health Organization, “Guidelines for the Regulatory Assessment of Medicinal Products for use in Self-Medication” (2000). Given the large number of individuals with uncontrolled triglyceride levels, providing access to OTC high purity concentrated fish oils could provide significant societal health benefits.

However, switching distribution of a pharmaceutical from prescription-only to OTC creates a significant risk that the patient population will be unable to appropriately self-select themselves for safe use of the pharmaceutical use and then self-medicate using the drug in a responsible manner. The manifestations embodied within these concerns include incorrect self-diagnosis, incorrect drug-qualification, unrecognized drug-drug interactions (DDI), unanticipated adverse drug reactions and/or side-effects, improper dosing and/or administration, masking of a disease, addiction, inappropriate drug dependency, substance abuse, and patient delay in seeking necessary medical attention. Ruiz et al., Current Drug Safety, 5(4):315 (2010).

In order to ensure the safety of OTC distribution of concentrated fish oil, prospective patients must effectively self-select themselves for the drug. Recent studies, however, found that many prospective patients do not pay consistent attention to guidelines printed on the packaging of OTC drugs, to ensure safe and responsible use. PR Newswire Association, “Americans Should Pay More Attention to Over-the-Counter (OTC) medicine Labels According to New Survey,” Oct. 15 (2015) (citing McNeil Consumer Healthcare research). According to these studies, 40% of prospective patients consider the directions as just guidelines and 80% of patients do not re-read the label of an OTC medicine they have used before. Even more troubling, only 58% of men surveyed found it very important to pay attention to restrictions on an OTC label.

Currently, there are two regulatory pathways for legal marketing of an OTC drug in the United States. In the first pathway, marketing occurs in compliance with an OTC drug monograph, that sets regulatory standards for non-prescription drugs that are not covered by human drug applications, e.g., a New Drug Application (NDA) or Abbreviated New Drug Application (ANDA). An OTC monograph is created as a result of a three phase OTC drug review by the FDA. In phase I of the review, an advisory review panel determines whether ingredients in the proposed OTC composition could be generally recognized as safe and effective for use in self-treatment. In the second pathway, marketing occurs under the authority of an approved product-specific new drug application (NDA), or an abbreviated new drug application (ANDA). In order to support an over-the-counter label for a drug for which regulatory approval is being sought through an NDA, a consumer research study is required to assess the consumer's ability to select and deselect themselves as appropriate users of the drug, based on the proposed labeling for the drug. Oliver, A., Regulatory Rapporteur, 10(3):4-9 (2013), which is incorporated by reference herein.

However, attempts at switching distribution of cardiovascular drugs having potentially far-reaching benefits for societal health, from prescription-only to an OTC model, have repeatedly failed, in large part due to concerns over inappropriate patient selection and medication. Possibly the best documented cases relate to statins used to treat high cholesterol.

For instance, Merck has had at least three applications for sale of over the counter lovastatin rejected by the FDA, in 2000, 2005, and 2007. In 2005, their proposal to permit over the counter sales of lovastatin was rejected by an expert advisory panel at the FDA in 2005. The panel was concerned by a marketing study performed to support the proposal in which approximately one third of 3316 customers who were offered the drug over the counter decided they would purchase the drug. After reviewing the data, the panel concluded that 45% of the purchases would have been inappropriate for a variety of reasons, including the age of the subject, the subject's lack of knowledge about their condition, and contraindications associated with their condition. Dyer O., B M J, 330(7484):164 (2005). In 2007, the board again concluded that the ability of consumers to appropriately self-select and to adequately comply with chronic MEVACOR® therapy without the intervention of a physician had not been demonstrated. Division of Metabolic and Endocrine Drug Products, 2005, “NDA 21-213 Non-prescription MEVACOR® 20 mg Joint Advisory Committee Meeting.”

Similarly, Pfizer announced in 2011 its intention to switch LIPITOR® from prescription-only to OTC status. Sett OTC bulletin, 16 Nov. 2011, page 7. However, they abandoned their attempt in 2014 when a phase 3 “actual use” trial, intended to simulate the OTC use of LIPITOR® (atorvastatin calcium) 10 mg, failed to meet its primary objectives on the basis that patient compliance with the direction to check their low-density lipoprotein cholesterol (LDL-C) level and, after checking their LDL-C level, take appropriate action based on their test results was unsatisfactory. Pfizer Inc., “Pfizer Reports Second-Quarter 20150Results,” (2015).

In fact, in the nearly two decades since Bristol-Myers Squibb and Merck & Co first failed in their attempts to switch PRAVACHOL® and lovastatin, respectively, to OTC, a statin has never been granted OTC status in the United States. This is despite that nearly 35 million American adults who are eligible for cholesterol-lowering medications, under the current guidelines, are not taking anything. Mercado et al., MMWR Morb Mortal Wkly Rep., 64(47): 1305-11 (2015).

The information disclosed in this Background section is only for enhancement of understanding of the general background of the invention and should not be taken as an acknowledgment or any form of suggestion that this information forms the prior art already known to a person skilled in the art.

### SUMMARY

Given the above background, what is needed in the art are systems and methods for qualifying a human subject for delivery of a concentrated fish oil-based pharmaceutical composition over-the-counter for lowering triglyceride levels, e.g., thereby treating or preventing hypertriglyceridemia and/or heart disease.

The present disclosure addresses the need in the art for systems and methods configured for qualifying a human subject for over-the-counter delivery of a concentrated fish oil-based pharmaceutical composition (e.g., omega-3-carboxylic acid) for lowering triglyceride levels, e.g., thereby treating or preventing hypertriglyceridemia and/or heart disease. In the present disclosure, systems and methods are provided for over-the-counter delivery of a concentrated fish oil-based pharmaceutical composition to a subject. Survey results from the subject are run against a first plurality of filters. When a filter in the first plurality is fired, the subject is deemed not qualified for delivery of the concentrated fish oil-based pharmaceutical composition. The survey results are also run against a second plurality of filters. When a respective filter in the second plurality is fired, the subject is provided with a corresponding warning. The method proceeds to a fulfillment process when no filter in the first plurality is fired and the subject has acknowledged each warning associated with each fired filter in the second plurality of filters. The fulfillment process stores the composition order, communicates a drug facts label for the concentrated fish oil-based pharmaceutical composition to the subject, and authorizes, upon subject confirmation that the label has been read, provision of the concentrated fish oil-based pharmaceutical composition to the subject.

Accordingly, one aspect of the present disclosure provides a method for qualifying a subject for over-the-counter delivery of a concentrated fish oil-based pharmaceutical composition for lowing the triglyceride level of the subject, e.g., thereby treating or preventing hypertriglyceridemia and/or heart disease. The method includes conducting a first survey of the subject in order to obtain a variety of survey results. In some embodiments, the survey results indicate one or more of: a triglyceride level of the subject, an age of the subject, a dietary status of the subject, a diabetes status of the subject, a thyroid status of the subject, whether the subject has a (e.g., a preexisting) liver problem, whether the subject has a pancreatic problem, whether the subject is one of pregnant, breastfeeding, or planning to become pregnant, whether the subject has a seafood allergy, and whether the subject is taking a medication the affects coagulation.

The method also includes running all or a portion of the survey results against a first plurality of filters of a first category class, corresponding to contraindications associated with the concentrated fish oil-based pharmaceutical composition. When a respective filter in the first plurality of filters is fired, the subject is deemed not qualified for delivery of the concentrated fish oil-based pharmaceutical composition, and the method is then terminated accordingly without delivery of the concentrated fish oil-based pharmaceutical composition to the subject. In some embodiments, the first plurality of filters includes one or more of a triglyceride level filter, an age filter, and a dietary filter.

The method also includes running all or a portion of the survey results against a second plurality of filters of a second category class, corresponding to risk factors associated with the concentrated fish oil-based pharmaceutical composition. When a respective filter in the second plurality of filters is fired, the subject is provided with a warning corresponding to the respective filter. In some embodiments, the second plurality of filters includes one or more of: a diabetes filter, a thyroid disease filter, a liver disease filter, a pancreatic disease filter, a pregnancy filter, an allergy filter, and a coagulant medication filter. However, unlike filters in the first plurality of filters, filters in the second plurality of filters do not automatically terminate the process without delivery of the concentrated fish oil-based pharmaceutical composition to the subject.

The method continues by obtaining acknowledgment from the subject for the warning issued to the subject by any filter in the second plurality of filters.

The method continues by proceeding with a fulfillment process when no filter in the first plurality of filters has been fired and the subject has acknowledged each warning associated with each filter in the second plurality of filters that was fired.

In some embodiments, the fulfillment process includes storing an indication in a subject profile of an initial order for the concentrated fish oil-based pharmaceutical composition, communicating an over-the-counter drug label for concentrated fish oil-based pharmaceutical composition, and authorizing, upon confirmation from the subject that the over-the-counter drug label has been received and read, provision of the concentrated fish oil-based pharmaceutical composition to the subject.

In some embodiments, the concentrated fish oil-based pharmaceutical composition includes an omega-3-carboxylic acid pharmaceutical composition. In some embodiments, the concentrated fish oil-based pharmaceutical composition includes an omega-3 acid ethyl ester pharmaceutical composition, an ethyl eicosapentaenoic acid (EPA) pharmaceutical composition, or a krill oil pharmaceutical composition. In some embodiments, the concentrated fish oil-based composition includes a docosahexaenoic acid (DHA) pharmaceutical composition. In some embodiments, the concentrated fish oil-based composition includes a combination of an EPA pharmaceutical composition and a DHA pharmaceutical composition.

In one aspect, the present disclosure provides a method for qualifying a subject (e.g., a subject who was previously qualified to receive a provision of the concentrated fish oil-based pharmaceutical composition) for a re-order of the concentrated fish oil-based pharmaceutical composition (e.g., which is optionally performed in conjunction with a method for qualifying the subject for a first order of the concentrated fish oil-based pharmaceutical composition). The method includes a re-fulfillment procedure that includes conducting a second survey of the subject in order to obtain a second plurality of survey results. In some embodiments, the second plurality of survey results indicates one or more of: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation.

The method also includes running all or a portion of the second plurality of survey results against a third plurality of filters of the first category class, corresponding to contraindications associated with the concentrated fish-oil based pharmaceutical composition. When a respective filter in the third plurality of filters is fired, the subject is deemed not qualified for the concentrated fish oil-based pharmaceutical composition, and the re-fulfillment process is terminated without delivery of the concentrated fish oil-based pharmaceutical composition to the subject. In some embodiments, the third plurality of filters includes a dietary filter and a triglyceride level filter.

The method also includes running all or a portion of the second plurality of survey results against a fourth plurality of filters of the second category class, corresponding to risk factors associated with the metformin pharmaceutical composition. When a respective filter in the fourth plurality of filters is fired the subject is provided with a warning corresponding to the respective filter. In some embodiments, the fourth plurality of filters includes one or more of: a diabetes filter, a thyroid filter, a liver disease filter, a pancreatic disease filter, a pregnancy filter, and a coagulant medication filter.

The method continues by obtaining acknowledgment from the subject for the warning issued to the subject by any filter in the fourth plurality of filters. When the re-fulfillment process is not already terminated by the firing of a filter in the third plurality of filters and the subject has acknowledged each warning associated with each filter in the fourth plurality of filters that was fired, the method continues with a re-fulfillment procedure.

In some embodiments, the re-fulfillment procedure includes storing an indication in the subject profile of a re-order for the concentrated fish oil-based pharmaceutical composition, communicating an over-the-counter drug facts label for the concentrated fish oil-based pharmaceutical composition to the subject, and authorizing, upon confirmation from the subject that the over-the-counter drug facts label has been received and read, a re-order provision of the concentrated fish oil-based pharmaceutical composition to the subject.

In some embodiments, the concentrated fish-oil based pharmaceutical composition includes an omega-3-carboxylic acid pharmaceutical composition.

### BRIEF DESCRIPTION OF THE DRAWINGS

FIG. 1 illustrates an exemplary system topology that includes a concentrated fish oil-based pharmaceutical composition over-the-counter (OTC) dispensing device for qualifying a human subject for over-the-counter delivery of a concentrated fish oil-based pharmaceutical composition for lowering triglyceride levels, a data collection device for collecting subject data, one or more user devices associated with human subjects, and one or more dispensary destinations for distributing the concentrated fish oil-based pharmaceutical composition over-the-counter, where the above-identified components are interconnected, optionally through a communications network, in accordance with an embodiment of the present disclosure.

FIG. 2 illustrates an example device for qualifying a human subject for delivery of a concentrated fish oil-based pharmaceutical composition over-the-counter to lower triglycerides, in accordance with various embodiments of the present disclosure.

FIGS. 3A and 3B collectively illustrate an example device associated with a human subject for qualifying the human subject for over-the-counter delivery of a concentrated fish oil-based pharmaceutical composition to lower triglycerides, in accordance with an embodiment of the present disclosure, where it will be appreciated that the example device of FIGS. 3A and 3B works in conjunction with the device of FIG. 2 to perform the methods illustrated in FIGS. 4 through 8 in some embodiments by, for instance providing the device of FIG. 2 with survey results and/or the results of firing filters of the present disclosure against such survey results but that, in alternative embodiments, the device of FIG. 2 performs all the methods of the present disclosure and the device of FIGS. 3A and 3B is not used. In still further alternative embodiments, the device of FIGS. 3A and 3B performs the methods of the present disclosure and the device of FIG. 2 is not used.

FIGS. 4A, 4B, 4C, 4D, 4E, 4F, and 4G collectively provide a flow chart of processes for qualifying a human subject for over-the-counter delivery of a concentrated fish oil-based pharmaceutical composition to lower triglycerides, where elements in dashed boxes are optional, in accordance with various embodiments of the present disclosure.

FIGS. 5A, 5B, 5C, 5D, 5E, 5F, and 5G illustrate example survey questions for obtaining survey results, in accordance with an embodiment of the present disclosure.

FIG. 6 illustrates feedback from a first survey, in accordance with an embodiment of the present disclosure.

FIGS. 7A, 7B, and 7C collectively illustrate an example method for qualifying a subject for an over-the-counter provision of a concentrated fish oil-based pharmaceutical composition, in accordance with an embodiment of the present disclosure.

FIGS. 8A, 8B, and 8C collectively illustrate an example method for qualifying a subject for a refill of an over-the-counter provision concentrated fish oil-based pharmaceutical composition, in accordance with an embodiment of the present disclosure.

In the figures, reference numbers refer to the same or equivalent parts of the present invention throughout the several figures of the drawing.

### DETAILED DESCRIPTION

Hypertriglyceridemia is a growing health problem, in the United States and worldwide. Although hypertriglyceridemia can be effectively treated and/or prevented using established pharmaceutical compositions, access to these drugs is hindered by to the requirement for a prescription, as many individuals do not have adequate access and/or avoid the healthcare system for a variety of reasons. Accordingly, many people are not managing their hypertriglyceridemia or heart disease conditions appropriately. While over-the-counter alternatives to these prescription pharmaceuticals would increase access to these compositions, thereby improving population management of hypertriglyceridemia and heart disease around the world, patients often have difficulty self-selecting themselves for an appropriate over-the-counter medication. Because inappropriate use of these drugs can result in ineffective treatment and/or serious side-effects, better methods for selecting for, and treating patients with, other-the-counter hypertriglyceridemia medications are needed. The present disclosure provides, among other aspects, methods, systems, and computer readable media that solve these problems.

Reference will now be made in detail to implementations, examples of which are illustrated in the accompanying drawings. In the following detailed description of implementations, numerous specific details are set forth in order to provide a thorough understanding of the present invention. However, it will be apparent to one of ordinary skill in the art that the present invention may be practiced without these specific details.

It will also be understood that, although the terms first, second, etc. may be used herein to describe various elements, these elements should not be limited by these terms. These terms are only used to distinguish one element from another. For example, a first filter could be termed a second filter, and, similarly, a second filter could be termed a first filter, without departing from the scope of the present disclosure. The first filter and the second filter are both filters, but they are not the same filter.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “over-the-counter” means to provide by retail purchase, subject to the constraints disclosed herein, but without a prescription or license from a physician or medical practitioner.

As used herein, the term “pharmaceutical compound” refers to any physical state of a material. Pharmaceutical compounds include capsules, tablets, liquids, topical formulations, and inhaled formulations.

As used herein, the term “contraindication” refers to a condition that makes a treatment, e.g., over-the-counter use of a concentrated fish oil-based pharmaceutical composition, inadvisable. Contraindications include physical characteristics of a subject, e.g., pregnancy or liver disease, and contemporaneous drug use, e.g., concentrated fish oil-based pharmaceutical composition use. In the present context, identification of a contraindication fires a filter of a first category class, which prevents authorizing provision of a concentrated fish oil-based pharmaceutical composition, in accordance with some implementations of the methods, systems, and software disclosed herein.

As used herein, the term “risk factor” refers to a condition that makes a treatment, e.g., over-the-counter use of a concentrated fish oil-based pharmaceutical composition, possibly inadvisable. Risk factors include physical characteristics of a subject, e.g., a triglyceride level reading, and contemporaneous drug use, e.g., use of a coagulant medication. In the present context, identification of a risk factor fires a filter of a second category class, which prevents authorizing provision of a concentrated fish oil-based pharmaceutical composition without confirmation that the subject has discussed the risk factor with a medical professional, in accordance with some implementations of the methods, systems, and software disclosed herein.

As used herein, “drug interactions,” e.g., with a concentrated fish oil, include pharmacokinetic drug interactions and pharmacodynamics drug interactions. Generally, a pharmacokinetic drug interaction is an interaction between two drugs (e.g., a concentrated fish oil and a second drug) that result in alterations in the absorption, transport, distribution, metabolism, and/or excretion of either drug. Generally, a pharmacokinetic drug interaction is an interaction between two drugs (e.g., a concentrated fish oil and a second drug) that result in a direct change in the effect of either drug. For a more comprehensive summary of pharmacokinetic drug interactions and pharmacodynamics drug interactions, see, Cascorbi, I, Dtsch Arztebl Int., 109(33-34):546-55 (2012), the content of which is hereby incorporated by reference.

In the context of the present disclosure, classification of a condition as either a contraindication or a risk factor is specific to a particular identity and dose of a concentrated fish oil-based pharmaceutical composition being authorized for over-the-counter use. Classification of a particular condition, e.g., contemporaneous concentrated fish oil-based pharmaceutical composition use, may vary between different concentrated fish oil-based pharmaceutical compositions (e.g., it may be classified as a contraindication for a first concentrated fish oil, a risk factor for a second concentrated fish oil, and/or neither for a third concentrated fish oil-based). Likewise, a particular condition may be classified as a contraindication for use of a particular concentrated fish oil at a first over-the-counter dosage, classified as a risk factor for the same particular concentrated fish oil at a second (e.g., lower) over-the-counter dosage, and/or classified as neither for the same particular concentrated fish oil at a third (e.g., lowest) over-the-counter dosage.

As used herein, whether a subject “has developed” a condition since receiving their last provision of a concentrated fish oil-based composition refers to both conditions that are new to the subject, i.e., a condition that the subject did not have at the time they received their last provision of the gliflozin Sodium-Glucose Cotransport 2 inhibitor, and conditions that have been newly diagnosed, regardless of whether the condition existed when the subject received their last provision of the gliflozin Sodium-Glucose Cotransport 2 inhibitor, i.e., a condition that the subject was not aware of when they received their last provision of the concentrated fish oil-based composition.

In one aspect of the present disclosure a survey of a subject is conducted to obtain survey results in order to determine if the subject qualifies for an over-the-counter (OTC) concentrated fish oil-based pharmaceutical composition for lowering triglyceride levels, e.g., thereby treating or preventing hypertriglyceridemia and/or heart disease. The survey results are used as the basis for running filters of a first category class. If the triggering conditions of any of the filters in the first category class are fired, the subject does not qualify for the OTC concentrated fish oil-based pharmaceutical composition. The survey results are also used as the basis for running filters of a second category class. If the triggering conditions of any of the filters in the second category class are fired, the subject is provided with warning messages associated with the respective filters of the second category class that have been fired. If none of the filters in the first category class are fired and the subject successfully addresses the warning messages associated with the respective filters of the second category class that have been fired a fulfillment process is initiated for OTC delivery of the concentrated fish oil-based pharmaceutical composition.

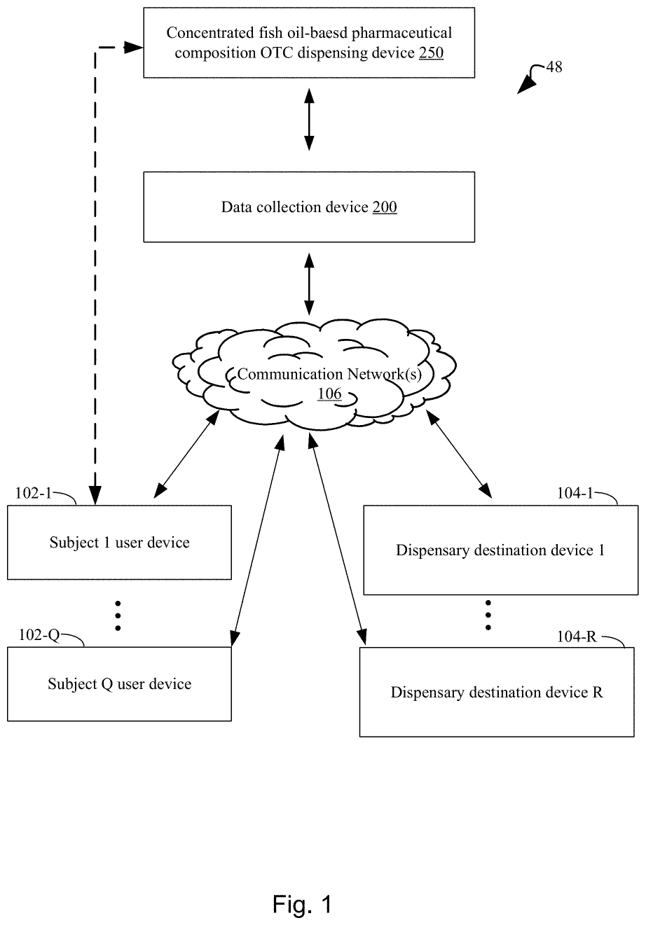

FIG. 1 illustrates an example of an integrated system 48 for conducting one or more surveys of subjects in order to qualifying the subjects for OTC delivery of a concentrated fish oil-based pharmaceutical composition. The integrated system 48 includes one or more connected user devices 102. The user devices 102 are configured for entering survey data and making requests for the concentrated fish oil-based pharmaceutical composition. The system 48 also includes one or more dispensary destination devices 104 that are configured to receive instructions in order to provide the concentrated fish oil-based pharmaceutical composition to qualifying subjects. Furthermore, the system 48 includes a concentrated fish oil-based over-the-counter (OTC) dispensing device 250 and one or more data collection devices 200 that are configured for collecting subject data.

Throughout the present disclosure, the data collection device 200 and the concentrated fish oil-based pharmaceutical composition OTC dispensing device 250 will be referenced as separate devices solely for purposes of clarity. That is, the disclosed functionality of the data collection device 200 and the disclosed functionality of the concentrated fish oil-based pharmaceutical composition OTC dispensing device 250 are contained in separate devices as illustrated in FIG. 1. However, it will be appreciated that, in fact, in some embodiments, the disclosed functionality of the data collection device 200 and the disclosed functionality of the concentrated fish oil-based pharmaceutical composition OTC dispensing device 250 are contained in a single device.

With the integrated system 48, survey results from the subjects are run against a first plurality of filters (e.g., filter 216-1, filter 216-2, filter 216-4, etc.) When a filter in the first plurality of filters (e.g., filter 216) is fired for a respective subject, the respective subject is deemed not qualified for the concentrated fish oil-based pharmaceutical composition. The survey results are also run against a second plurality of filters (e.g., filter 222-1, filter 222-2, filter 222-6, etc.) When a respective filter in the second plurality is fired for a respective subject, the respective subject is provided with a warning (e.g., filter warning 226) associated with the respective filter. In some embodiments the survey results are run against the first plurality of filters and the second plurality of filters concurrently. In some embodiments the survey results are run against the first plurality of filters and then against the second plurality of filters. The method enabled by the integrated system 48 proceeds to a fulfillment process when no filter in the first plurality fires and the subject has acknowledged, or otherwise successfully addressed, each warning associated with each filter in the second plurality of filters that fired. As part of the fulfillment process, the composition order is stored (e.g., in a user profile 234 associated with the subject to receive the drug), a drug facts label (e.g., drug facts label 230) for the concentrated fish oil is communicated to the qualifying subject. Upon subject confirmation that the label has been read, authorization is granted to dispense the concentrated fish oil.

Referring to FIG. 1, the concentrated fish oil-based pharmaceutical composition OTC dispensing device 250 qualifies a subject for over-the-counter delivery of a concentrated fish oil-based pharmaceutical composition for lowering triglycerides, e.g., thereby treating or preventing heart disease. To accomplish this, the data collection device 200, which is in electrical communication with the concentrated fish oil-based pharmaceutical composition OTC dispensing device 250, receives survey results originating from one or more user devices 102 associated with corresponding subjects. In some embodiments, the data collection device 200 receives such survey results directly from the user devices 102. For instance, in some embodiments the data collection device 200 receives this data wirelessly through radio-frequency signals. In some embodiments, such signals are in accordance with an 802.11 (Wi-Fi), Bluetooth, or ZigBee standard. In some embodiments, the data collection device 200 receives such data directly, analyzes the data, and passes the analyzed data to the concentrated fish oil-based pharmaceutical composition OTC dispensing device 250.

In some embodiments, the data collection device 200 and/or the concentrated fish oil-based pharmaceutical composition OTC dispensing device 250 is not proximate to the subject and/or does not have wireless capabilities or such wireless capabilities are not used for the purpose of acquiring survey results. In some embodiments, a communication network 106 may be used to survey questions (e.g., survey questions 208, 212) from the concentrated fish oil-based pharmaceutical composition OTC dispensing device 250 to user devices 102 and the answers to such survey questions from the user devices 102 to the data collection device 200 and/or the concentrated fish oil-based pharmaceutical composition OTC dispensing device 250. Further, in some embodiments the communication network 106 is used to communicate authorization to dispense the concentrated fish oil from the concentrated fish oil-based pharmaceutical composition OTC dispensing device 250 to dispensary destination devices 104.

Examples of networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 48 are possible. For instance, rather than relying on a communications network 106, the one or more user devices 102 and the one or more dispensary destination devices 104 may communicate directly to the data collection device 200 and/or the concentrated fish oil-based pharmaceutical composition OTC dispensing device 250. Further, the data collection device 200 and/or concentrated fish oil-based pharmaceutical composition OTC dispensing device 250 may constitute a portable electronic device, a server computer, or in fact constitute several computers that are linked together in a network, be a virtual machine in a cloud computing context, be a container in a cloud computer context, or a combination thereof. As such, the exemplary topology shown in FIG. 1 merely serves to describe the features of an embodiment of the present disclosure in a manner that will be readily understood to one of skill in the art.

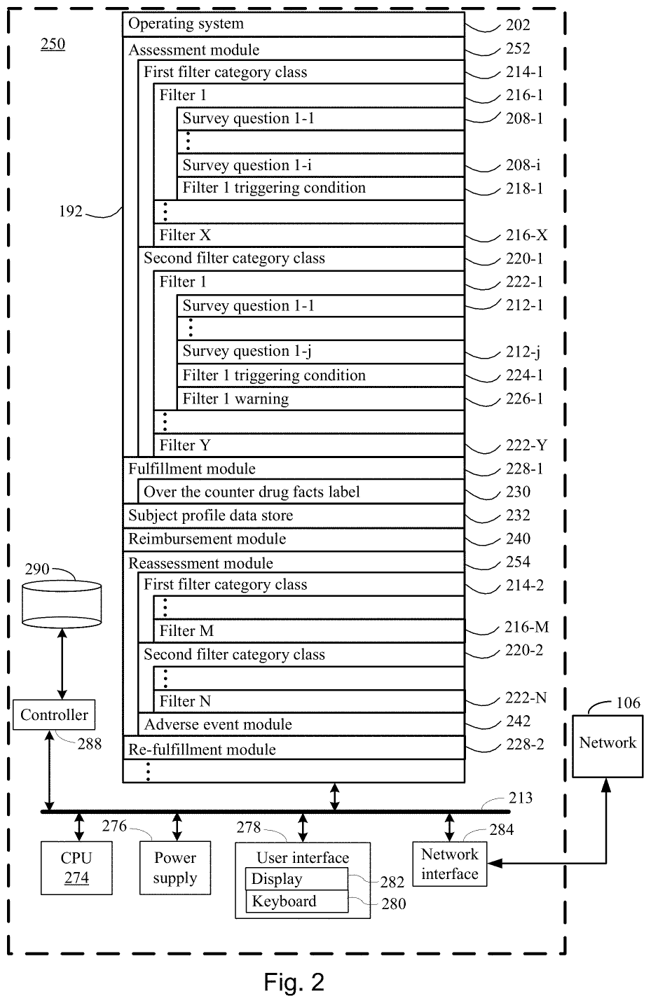

Turning to FIG. 2 with the foregoing in mind, an exemplary concentrated fish oil-based pharmaceutical composition OTC dispensing device 250 configured for determining whether a subject is qualified for OTC delivery of a concentrated fish oil is depicted. Referring to FIG. 2, in typical embodiments, the concentrated fish oil-based pharmaceutical composition OTC dispensing device 250 comprises one or more computers. For purposes of illustration in FIG. 2, the concentrated fish oil-based pharmaceutical composition OTC dispensing device 250 is represented as a single computer that includes all of the functionality for qualifying a human subject for over-the-counter delivery of a concentrated fish oil-based pharmaceutical composition for lowering triglycerides (e.g., treating or preventing heart disease). However, the present disclosure is not limited thereto. In some embodiments, the functionality for qualifying a human subject for over-the-counter delivery of a concentrated fish oil-based pharmaceutical composition for lowering triglycerides (e.g., treating or preventing heart disease) is spread across any number of networked computers and/or resides on each of several networked computers, is hosted on one or more virtual machines at a remote location accessible across the communications network 106, and/or is hosted on one or more containers at a remote location accessible across the communications network 106. One of skill in the art will appreciate that any of a wide array of different computer topologies are used for the application and all such topologies are within the scope of the present disclosure.

The concentrated fish oil-based pharmaceutical composition OTC dispensing device 250 of FIG. 2 is configured to conduct a first survey (e.g., using assessment module 252 to perform an initial qualification of the subject for provision of a concentrated fish oil-based pharmaceutical composition) and/or a second survey (e.g., using reassessment module 254 to perform a re-qualification of the subject for provision of a concentrated fish oil-based pharmaceutical composition). The first survey (e.g., the assessment) comprises a variety of questions 208, 212 associated with filters 216, 222 within a plurality of filters of the first filter category class 214-1 and a plurality of filters in the second filter category class 220-1, respectively. Answers to the questions in the first survey received by the device are run against filters of a first category class 214-1 and filters of a second category class 220-1 within the first and second pluralities of filters 216, 222, respectively. Similarly, the second survey (e.g., the re-assessment) also comprises a variety of questions associated with filters 216, 222 within a plurality of filters of a first category class 214-2 and a plurality of filters of a second category class 220-2, respectively. Answers to the questions in the second survey received by the device are run against filters of a first category class 216-2 and filters of a second category class 220-2, e.g., within the first and second pluralities of filters, respectively. Filters 216 of the first filter category class 214 are configured to terminate the qualification process when fired. Filters 222 of the second filter category class 220 are configured to provide the subject with a warning associated with a corresponding survey question. In other words, the device of FIG. 2 is configured to accumulate results from a survey (e.g., survey questions 208 and survey questions 212) and run the results against corresponding filters (e.g., filters 216 and filters 222, respectively) in order to determine if a subject is qualified for OTC delivery of a concentrated fish oil-based pharmaceutical composition.

In the present disclosure, a plurality of filters refers to a series, or set, or filters in either the first filter category class or the second category class. For instance, in some embodiments, a plurality of filters of the first filter category class 214 can comprise any subset of filters 216 of the first filter category class. As an example, in some embodiments a plurality of filters of the first category class comprises filters 216-1, 216-2, 216-3, . . . , 216-i, or any combination thereof. Similarly, a plurality of filters of the second filter category class 220 can comprise any set of filters 222 of the second filter category class. Moreover, in some embodiments a plurality of filters of the second category class comprises filters 222-1, 222-2, 222-3, . . . , 222-i, or any combination thereof

Continuing to refer to FIG. 2, in some embodiments, the dispensing device 250 comprises one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the concentrated fish oil-based pharmaceutical composition OTC dispensing device 250 but that can be electronically accessed by the concentrated fish oil-based pharmaceutical composition OTC dispensing device 250 over an Internet, intranet, or other form of network or electronic cable (illustrated as element 106 in FIG. 2) using network interface 284.

In some embodiments, the memory 192 of the concentrated fish oil-based pharmaceutical composition OTC dispensing device 250 stores one or more of:
operating system 202 that includes procedures for handling various basic system services;
assessment module 252 for qualifying a subject for an initial over-the-counter delivery of a concentrated fish oil-based pharmaceutical composition for lowering triglycerides, e.g., thereby treating or preventing heart disease, by communicating survey questions, obtaining results therefrom, and applying the results to qualifying filters, the assessment module including:
   a first filter category class 214-1, including filters 216 (e.g., a first plurality of filters), each respective filter 216 in the first filter category class 214-1 is associated with one or more survey questions 208 and one or more triggering conditions 218;
   a second filter category class 220-1, including filters 222 (e.g., a second plurality of filters), each respective filter 222 in the second filter category class 220-1 is associated with one or more survey questions 208, triggering conditions 224, and warnings 226;
a fulfillment module 228-1 for executing a fulfillment process when no filter 216 in the first filter category class 214-1 has been fired for a subject and the subject has acknowledged each warning 226 associated with each filter 222 in the second filter category class 220-1 that was fired as a result of answers by the subject to the survey questions 208, where the fulfillment process includes communicating an over-the-counter drug facts label 230 for the concentrated fish oil-based pharmaceutical composition to the subject and receiving confirmation from the subject that the over-the-counter drug facts label has been received and read;
a reassessment module 254 for qualifying a subject for a subsequent over-the-counter delivery of a concentrated fish oil-based pharmaceutical composition for lowering triglycerides, e.g., thereby treating or preventing heart disease, by communicating survey questions, obtaining results therefrom, and applying the results to qualifying filters, the assessment module including:
   a first filter category class 214-2, including filters 216 (e.g., a third plurality of filters), each respective filter 216 in the first filter category class 214-2 is associated with one or more survey questions 208 and one or more triggering conditions 218;
   a second filter category class 220-2, including filters 222 (e.g., a second plurality of filters), each respective filter 222 in the second filter category class 220-2 is associated with one or more survey questions 208, triggering conditions 224, and warnings 226;
a re-fulfillment module 228-2 for executing a re-fulfillment process when no filter 216 in the first filter category class 214-2 has been fired for a subject and the subject has acknowledged each warning 226 associated with each filter 222-2 in the second filter category class 220 that was fired as a result of answers by the subject to the survey questions 212, where the fulfillment process includes communicating an over-the-counter drug facts label 230 for the concentrated fish oil-based pharmaceutical composition to the subject and receiving confirmation from the subject that the over-the-counter drug facts label has been received and read;
a subject profile data store 232 comprising a user profile 234 for each of a plurality of subjects, each respective user profile 234 including information (e.g., shipping information, billing information, biometric information, etc.) about a corresponding subject in the plurality of subjects, an initial order date and destination 236, and any re-order date and the destination 238 for the concentrated fish oil-based pharmaceutical composition made by the corresponding subject using the concentrated fish oil-based pharmaceutical composition OTC dispensing device 250;
an adverse event module 242 for identifying and aggregating records of adverse events associated with a plurality of subjects, e.g., corresponding to the firing of a filter 216 in the first filter category class 214-2 during a re-fulfillment process;
a reimbursement module 240 for determining eligibility and/or communicating an insurance claim associated with delivery of the concentrated fish oil, e.g., based on insurance information stored in a respective user profile 234.

In some embodiments, the assessment module 252, reassessment module 254, and/or fulfillment module 228 are accessible within any browser (e.g., phone, tablet, laptop/desktop, or smartwatch). In some embodiments the assessment module 252, reassessment module 254, and/or fulfillment module 228 run on native device frameworks, and is available for download onto a user device 102 running an operating system 202 such as Android, iOS, or WINDOWS.

In some implementations, one or more of the above identified data elements or modules (e.g., assessment module 252, fulfillment module 228-1, etc.) of the concentrated fish oil-based pharmaceutical composition OTC dispensing device 250 for qualifying a human subject for over-the-counter delivery of a concentrated fish oil-based pharmaceutical composition for lowering triglycerides (e.g., treating or preventing heart disease) are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments the memory 192 and/or 290 stores additional modules and data structures not described above.

In some embodiments, a concentrated fish oil-based pharmaceutical composition OTC dispensing device 250 for qualifying a human subject for over-the-counter delivery of a concentrated fish oil-based pharmaceutical composition for lowering triglyceride levels is a smart phone (e.g., an iPhone, Blackberry, etc.), a laptop, a tablet computer, a desktop computer, a smart watch, or another form of electronic device (e.g., a gaming console). In some embodiments, the concentrated fish oil-based pharmaceutical composition OTC dispensing device 250 is not mobile. In some embodiments, the concentrated fish oil-based pharmaceutical composition OTC dispensing device 250 is mobile.

In some embodiments, the concentrated fish oil-based pharmaceutical composition OTC dispensing device 250 is not a smart phone but rather is a tablet computer, desktop computer, emergency vehicle computer, or other form or wired or wireless networked device. In the interest of brevity and clarity, only a few of the possible components of the concentrated fish oil-based pharmaceutical composition OTC dispensing device 250 are shown in FIG. 2 in order to better emphasize the additional software modules that are installed on the concentrated fish oil-based pharmaceutical composition OTC dispensing device 250.

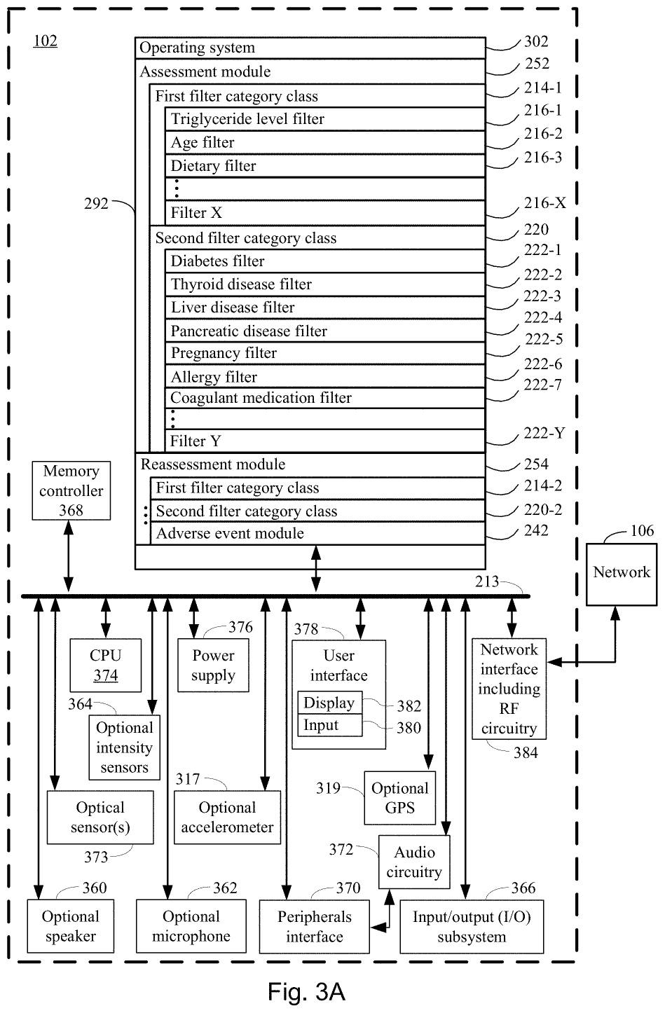

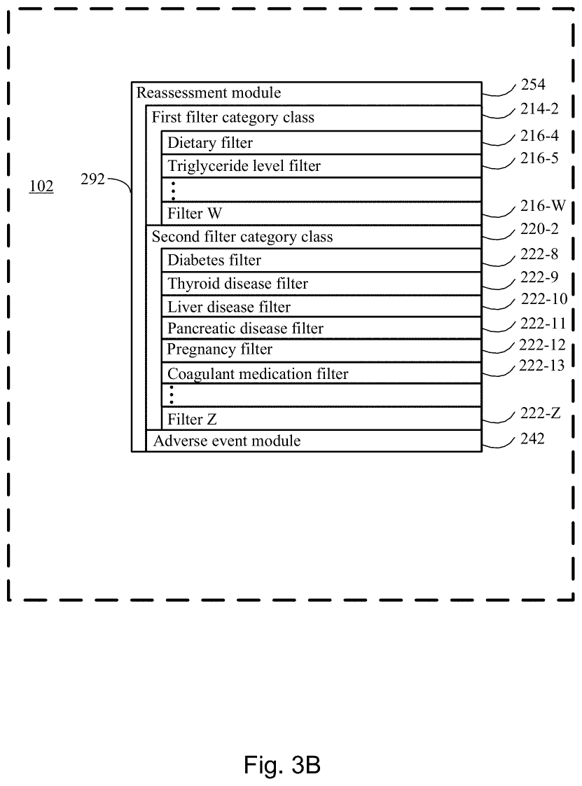

FIG. 3 provides a description of a user device 102 that can be used with the present disclosure. The user device 102 illustrated in FIG. 3 has one or more processing units (CPU's) 374, peripherals interface 370, memory controller 368, a network or other communications interface 384, a memory 392 (e.g., random access memory), a user interface 378, the user interface 378 including a display 382 and input 380 (e.g., keyboard, keypad, touch screen), an optional accelerometer 317, an optional GPS 319, optional audio circuitry 372, an optional speaker 360, an optional microphone 362, one or more optional intensity sensors 364 for detecting intensity of contacts on the user device 102 (e.g., a touch-sensitive surface such as a touch-sensitive display system 382 of the user device 102), an optional input/output (I/O) subsystem 366, one or more optional optical sensors 373, one or more communication busses 313 for interconnecting the aforementioned components, and a power supply 376 for powering the aforementioned components.

In some embodiments, the input 380 is a touch-sensitive display, such as a touch-sensitive surface. In some embodiments, the user interface 378 includes one or more soft keyboard embodiments. The soft keyboard embodiments may include standard (e.g., QWERTY) and/or non-standard configurations of symbols on the displayed icons.

The user device 102 illustrated in FIG. 3 optionally includes, in addition to accelerometer(s) 317, a magnetometer (not shown) and a GPS 319 (or GLONASS or other global navigation system) receiver for obtaining information concerning the location and orientation (e.g., portrait or landscape) of the user device 102 and/or for determining an amount of physical exertion by the subject.

It should be appreciated that the user device 102 illustrated in FIG. 3 is only one example of a multifunction device that may be used for performing a survey (e.g., first survey 206) in order to qualify for over-the-counter delivery of a concentrated fish oil-based pharmaceutical composition to lower triglycerides, and that the user device 102 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Memory 392 of the user device 102 illustrated in FIG. 3 optionally includes high-speed random access memory and optionally also includes non-volatile memory, such as one or more magnetic disk storage devices, flash memory devices, or other non-volatile solid-state memory devices. Access to memory 392 by other components of the concentrated fish oil-based pharmaceutical composition OTC dispensing device 250, such as CPU(s) 374 is, optionally, controlled by the memory controller 368. In some embodiments, the memory 392 of the user device 102 illustrated in FIG. 3 optionally includes:
an operating system 302 that includes procedures for handling various basic system services;
the assessment module 252 described above in conjunction with the concentrated fish oil-based pharmaceutical composition OTC dispensing device 250;
the first category class 214 described above in conjunction with the concentrated fish oil-based pharmaceutical composition OTC dispensing device 250 further comprising a triglyceride level filter 216-1, an age filter 216-2, a dietary filter 216-3, and a second allergy filter 216-4; and
the second category class 220 described above in conjunction with the concentrated fish oil-based pharmaceutical composition OTC dispensing device 250 comprising a diabetes filter 222-1, a thyroid disease filter 222-2, a liver disease filter 222-3, a pancreatic disease filter 222-4, a pregnancy filter 222-5, a second allergy filter 226-6, and a coagulant medication filter 222-7;

In some embodiments, the optional accelerometer 317, optional GPS 319, and/or magnetometer (not shown) of the user device 102 or such components are used to recommend to qualifying subjects one or more suitable destinations for delivery of the concentrated fish oil-based pharmaceutical composition over-the-counter. In some embodiments, the GPS 319 is used to determine if a subject is geographically restricted for OTC delivery of the concentrated fish oil-based pharmaceutical composition. Geographical restrictions include a subject residing outside of delivery or shipping regions, marketing restrictions, and/or government regulations.

The peripherals interface 370 can be used to couple input and output peripherals of the device to CPU(s) 374 and memory 392. The one or more processors 374 run or execute various software programs and/or sets of instructions stored in memory 392, such as the survey module 204, to perform various functions for the user device 102 and to process data.

In some embodiments, the peripherals interface 370, CPU(s) 374, and memory controller 368 are, optionally, implemented on a single chip. In some other embodiments, they are implemented on separate chips.

RF (radio frequency) circuitry of network interface 384 receives and sends RF signals, also called electromagnetic signals. In some embodiments, the survey module 204, survey questions 208/212, answers to survey questions 208/212, and/or the over-the-counter drug facts label 230 are communicated to the subject device 102 using this RF circuitry. In some embodiments, the RF circuitry 384 converts electrical signals to/from electromagnetic signals and communicates with communications networks and other communications devices and/or the data collection device 200 and/or the concentrated fish oil-based pharmaceutical composition OTC dispensing device 250 via the electromagnetic signals. The RF circuitry 384 optionally includes well-known circuitry for performing these functions, including an antenna system, an RF transceiver, one or more amplifiers, a tuner, one or more oscillators, a digital signal processor, a CODEC chipset, a subscriber identity module (SIM) card, memory, and so forth. RF circuitry 384 optionally communicates with the communication network 106. In some embodiments, the circuitry 384 does not include RF circuitry and, in fact, is connected to the network 106 through one or more hard wires (e.g., an optical cable, a coaxial cable, or the like).

In some embodiments, the audio circuitry 372, the optional speaker 360, and the optional microphone 362 provide an audio interface between the subject and the user device 102. The audio circuitry 372 receives audio data from the peripherals interface 370, converts the audio data to electrical signals, and transmits the electrical signals to the speaker 360. The speaker 360 converts the electrical signals to human-audible sound waves. In some embodiments, the speaker 260 converts the electrical signals to human-inaudible sound waves. The audio circuitry 372 also receives electrical signals converted by the microphone 362 from sound waves. The audio circuitry 372 converts the electrical signal to audio data and transmits the audio data to peripherals interface 370 for processing. Audio data is, optionally, retrieved from and/or transmitted to the memory 392 and/or the RF circuitry 384 by the peripherals interface 370.

In some embodiments, the power supply 376 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

In some embodiments, the user device 102 optionally also includes one or more optical sensors 373. The optical sensor(s) 373 optionally include charge-coupled device (CCD) or complementary metal-oxide semiconductor (CMOS) phototransistors. The optical sensor(s) 373 receive light from the environment, projected through one or more lens, and converts the light to data representing an image. The optical sensor(s) 373 optionally capture still images and/or video. In some embodiments, an optical sensor is located on the back of the user device 102, opposite the display 382 on the front of the user device 102, so that the input 380 is enabled for use as a viewfinder for still and/or video image acquisition. In some embodiments, another optical sensor 373 is located on the front of the user device 102 so that the subject's image is obtained (e.g., to verify the health, condition, or identity of the subject as part of qualifying the subject for over-the-counter delivery of a concentrated fish oil-based pharmaceutical composition for lowering triglyceride levels), to help diagnose a subject's condition remotely, or to acquire visual physiological measurements of the subject, etc.

As illustrated in FIG. 3, the user device 102 preferably comprises an operating system 302 that includes procedures for handling various basic system services. The operating system 302 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In some embodiments the user device 102 is a smart phone or a smart watch. In other embodiments, the user device 102 is not a smart phone or a smart watch but rather is a tablet computer, a desktop computer, an emergency vehicle computer, or other form or wired or wireless networked device. In the interest of brevity and clarity, only a few of the possible components of the user device 102 are shown in FIG. 3 in order to better emphasize the additional software modules that are installed on the user device 102.

While the system 48 disclosed in FIG. 1 can work standalone, in some embodiments it can also be linked with various electronic medical record systems to exchange information in any way.

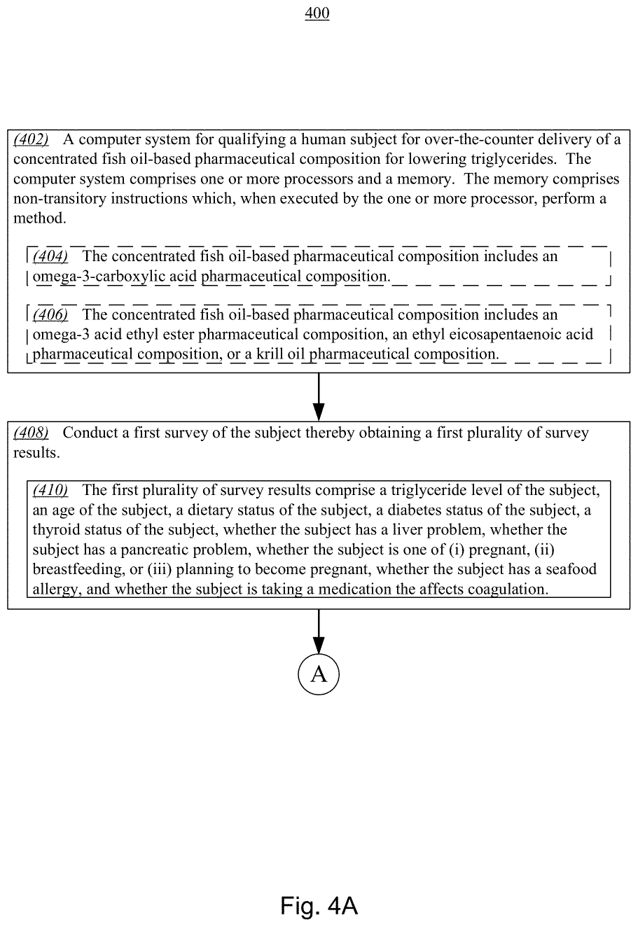

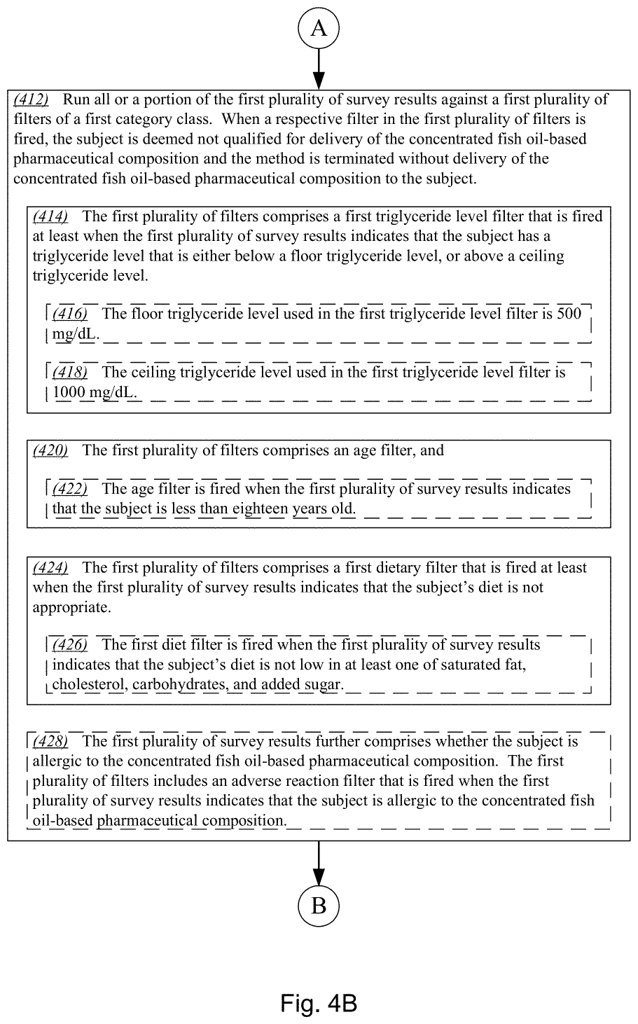

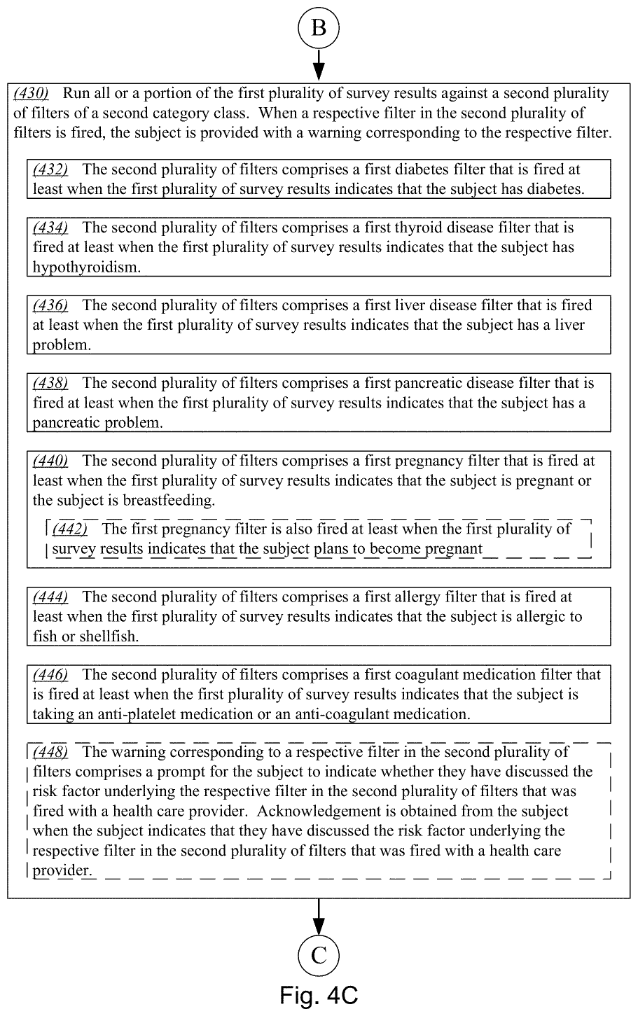

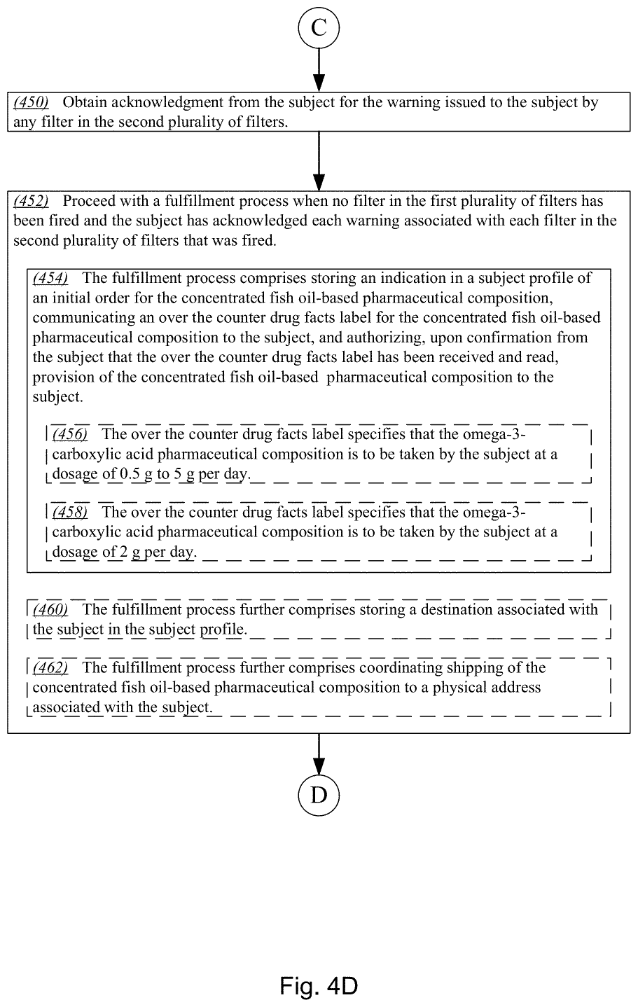

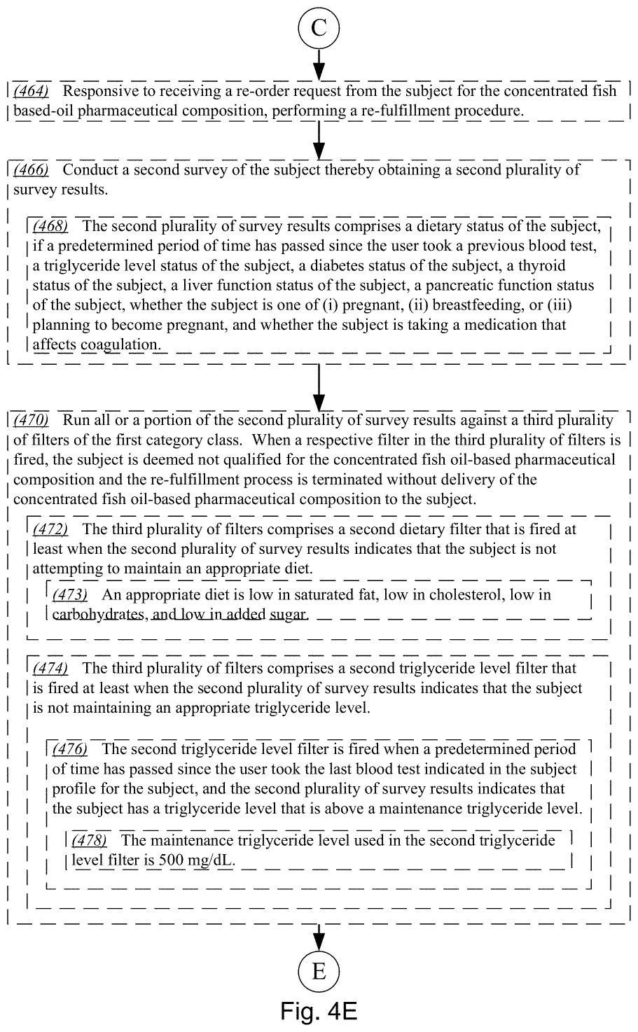

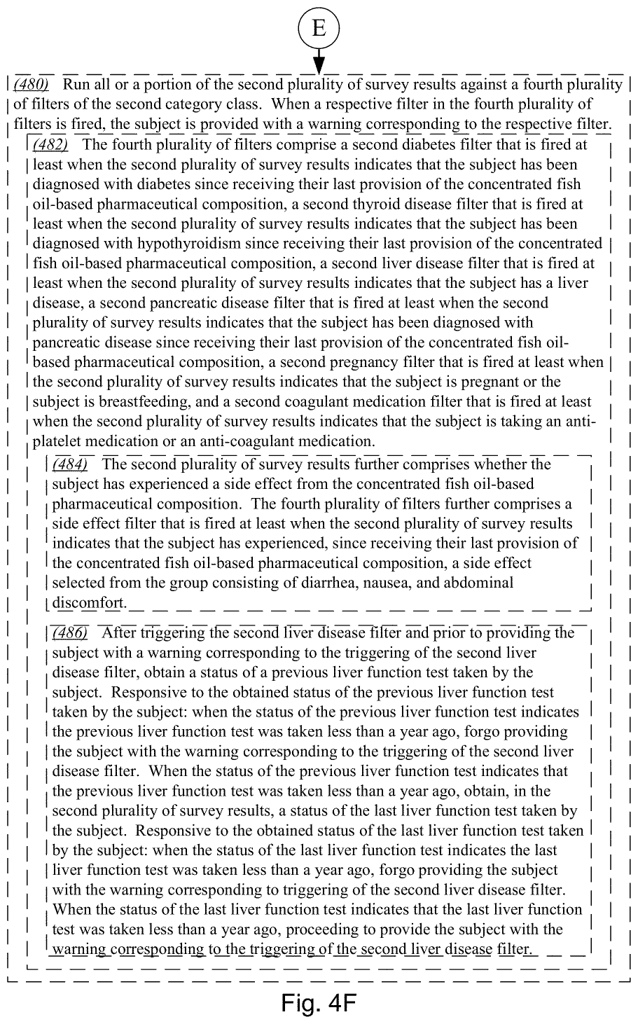

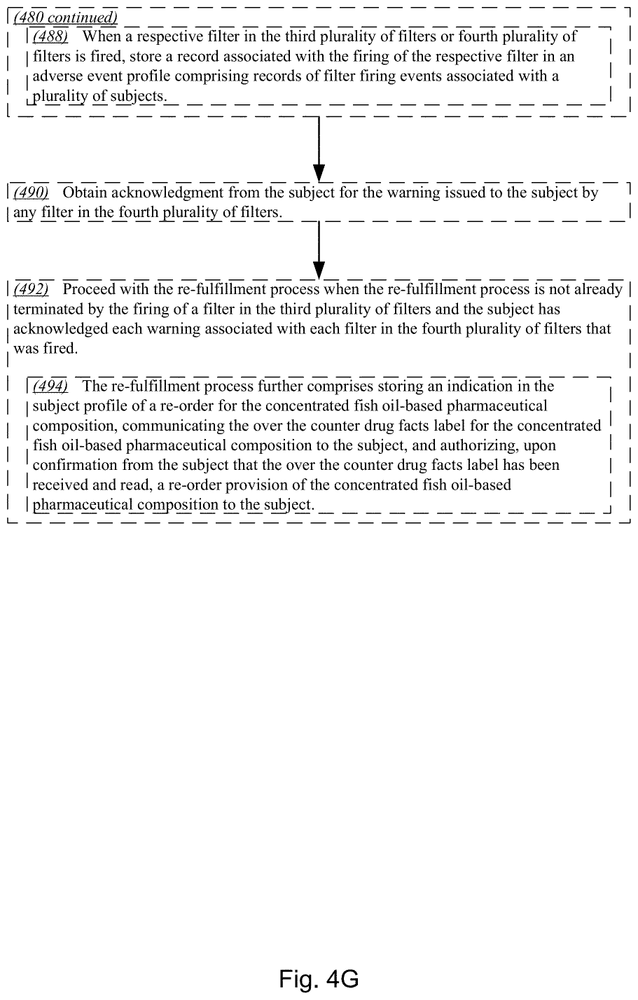

Now that details of a system 48 for qualifying a human subject for over-the-counter delivery of a concentrated fish oil-based pharmaceutical composition for lowering triglycerides (e.g., treating or preventing heart disease) have been disclosed, details regarding a method (400), including processes and features to be performed by the system, in accordance with an embodiment of the present disclosure, are disclosed with reference to FIGS. 4A through 4G. In some embodiments, such processes and features of the system are carried out by the assessment module 252, reassessment module 254, and/or fulfillment module 228 illustrated in FIGS. 2 and 3. In some embodiments, such processes and features of the system are carried out by the assessment module 252, reassessment module 254, fulfillment module 228-1, and/or re-fulfillment module 228-2 illustrated in FIGS. 2 and 3. In some embodiments, the assessment module 252, reassessment module 254, fulfillment module 228-1, and/or re-fulfillment module 228-1 are a single software module. In the flow chart, elements in dashed boxes are considered to be optional.

Blocks 402-412. Referring to block 402 of FIG. 4A, a goal of the present disclosure is to qualify subjects for over-the-counter delivery of a concentrated fish oil-based pharmaceutical composition for lowering triglyceride levels e.g., thereby, treating or preventing heart disease, using a computer system such as a concentrated fish oil-based pharmaceutical composition OTC dispensing device 250. The concentrated fish oil-based pharmaceutical composition OTC dispensing device (e.g., device 250) comprises one or more processors (e.g., processor 274) and a memory (e.g., memory 192 and/or 290). The memory stores non-transitory instructions that, when executed by the one or more processors, perform a method.

Referring to blocks 404 and 406, in some embodiments the concentrated fish oil-based pharmaceutical composition includes an omega-3-carboxylic acid (e.g., (4Z, 7Z, 10Z, 13Z, 16Z, 19Z)-docosa-4,7,10,13,16,19-hexaenoic acid;(4Z, 7Z, 10Z, 13Z, 16Z)-docosa-4,7,10,13,16-pentaenoic acid;(7Z, 10Z, 13Z, 16Z, 19Z)-docosa-7,10,13,16,19-pentaenoic acid;(6Z, 9Z, 12Z, 15Z, 18Z)-henicosa-6,9,12,15,18-pentaenoic acid;(5Z, 8Z, 11Z, 14Z, 17Z)-icosa-5,8,11,14,17-pentaenoic acid;(8Z, 11Z, 14Z, 17Z)-icosa-8,11,14,17-tetraenoic acid;(9Z, 12Z)-octadeca-9,12-dienoic acid;(6Z, 9Z, 12Z)-octadeca-6,9,12-trienoic acid;(9Z, 12Z, 15Z)-octadeca-9,12,15-trienoic acid). In some embodiments, the concentrated fish oil includes an omega-3 acid ethyl ester (e.g., ethyl (4Z, 7Z, 10Z, 13Z, 16Z, 19Z)-docosa-4,7,10,13,16,19-hexaenoate;ethyl (5Z, 8Z, 11Z, 14Z, 17Z)-icosa-5,8,11,14,17-pentaenoate), an ethyl eicosapentaenoic acid (EPA) (e.g., Ethyl (5Z, 8Z, 11Z, 14Z, 17Z)-eicosa-5,8,11,14,17-pentaenoate), and/or a krill oil composition.

In some embodiments, the concentrated fish oil-based pharmaceutical composition includes any compound disclosed in U.S. Pat. No. 7,652,068, entitled “Omega 3 Fatty Acid Formulations,” which is hereby incorporated by reference. In some embodiments, the concentrated fish oil-based pharmaceutical composition includes any compound disclosed in U.S. Pat. No. 4,895,725, entitled “Microencapsulation of Fish Oil,” which is hereby incorporated by reference.

In some embodiments, the concentrated fish oil-based pharmaceutical composition includes any compound disclosed in U.S. Pat. No. 8,446,013, entitled “Stable Pharmaceutical Composition and Methods of Using Same,” which is hereby incorporated by reference. In some embodiments, the concentrated fish oil-based pharmaceutical composition includes any compound disclosed in U.S. Pat. No. 5,502,077, entitled “Fatty Acid Composition,” which is hereby incorporated by reference.

In some embodiments, the concentrated fish oil-based pharmaceutical composition includes any compound disclosed in U.S. Pat. No. 8,188,146, entitled “Highly Purified Ethyl EPA and Other EPA Derivatives,” which is hereby incorporated by reference. In some embodiments, the concentrated fish oil-based pharmaceutical composition includes any compound disclosed in U.S. Pat. No. 8.298.554, entitled “Stable Pharmaceutical Composition and Methods of Using Same,” which is hereby incorporated by reference.

In some embodiments, in response to receiving a first request from a user to be qualified for provision of a concentrated fish oil-based pharmaceutical composition, the system creates a corresponding subject profile, e.g., containing biographic information about the subject, e.g., one or more of a subject name, date of birth, residence, delivery address, social security number, medical record number, insurance information, user name, identification password, etc. In some embodiments, the system registers a subject that has not previously received an over-the-counter provision of a concentrated fish oil-based pharmaceutical composition as a new user of the concentrated fish oil-based pharmaceutical composition, and the device will perform an initial assessment method for qualifying the subject for a provision of the concentrated fish oil-based pharmaceutical composition, e.g., regardless of whether the subject previously received a provision of a concentrated fish oil-based pharmaceutical composition via prescription.

In some embodiments, the system registers a subject that has previously received a provision of a concentrated fish oil-based pharmaceutical composition via prescription as a previous user of the concentrated fish oil-based pharmaceutical composition, and the device will perform a reassessment method for re-qualifying the subject for a provision of the concentrated fish oil-based pharmaceutical composition.

In some embodiments, where the subject previously received a provision of a different concentrated fish oil-based pharmaceutical composition via prescription, the system will perform a modified method for qualifying the subject for provision of the concentrated fish oil-based pharmaceutical composition that accounts for differences in the contraindications and risk factors of the two concentrated fish oil-based pharmaceutical compositions.

In some embodiments, in response to receiving a second or subsequent request from a user to be qualified for provision of a concentrated fish oil-based pharmaceutical composition, the system registers the subject as a returning customer, e.g., when the subject has previously received an over-the-counter provision of the concentrated fish oil-based and a corresponding subject profile 232 already exists for the subject.

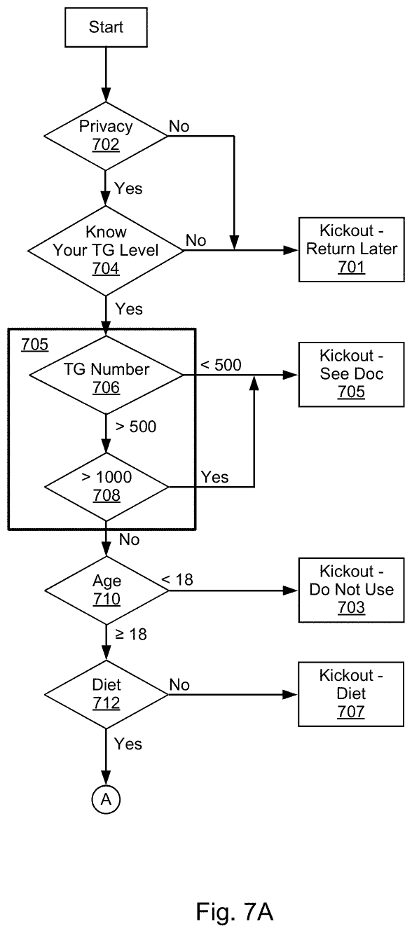

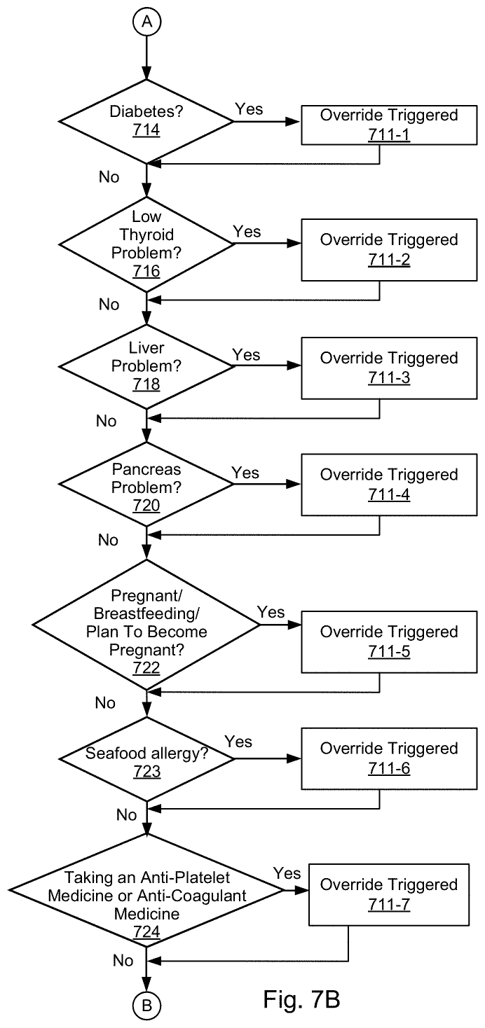

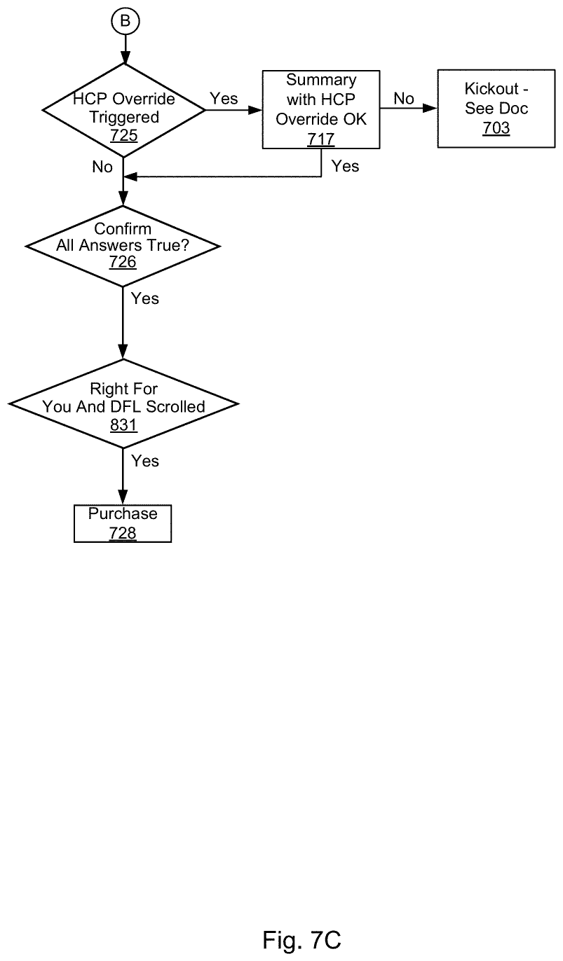

In some embodiments, prior to proceeding with the qualification or re-qualification method, the device prompts (702,704) the user to confirm that they have adequate privacy to provide sensitive medical information (e.g., prompt 702 in FIG. 7A) and/or that they are in possession of medical information required to complete the qualification process (e.g., prompt 704 to confirm that they have knowledge of their triglyceride level, in FIG. 7A)

Blocks 408-410. Referring to block 408 of FIG. 4A, the method includes conducting a first survey of the subject thereby obtaining a first plurality of survey results (e.g., in response to survey questions 208, 212, e.g., one or more of the survey questions set forth in Table 1). In some embodiments, the device transmits one or more survey questions to the user, prompting a response, and then receives a response to the one or more survey questions back from the subject. In some embodiments, the first survey results include, or at least indicate, some or all of the subject characteristics listed in Table 1. For example, in some embodiments, the first plurality of survey results includes, or at least indicates, 2, 3, 4, 5, 6, 7, 8, 9, 10, or all 11 of the characteristics listed in Table 1. In one embodiment, the first survey questions 208, 212 and results include at least characteristics 1-10 as provided in Table 1.

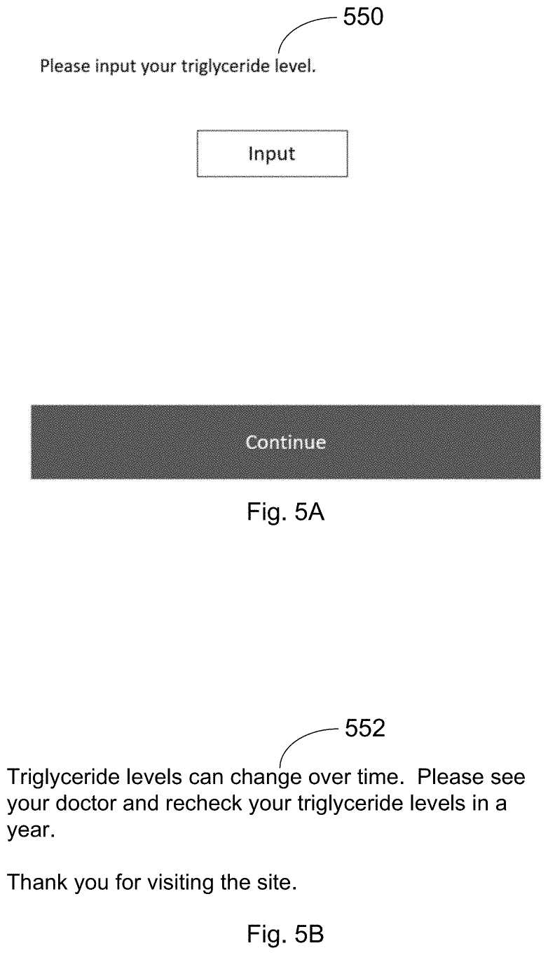

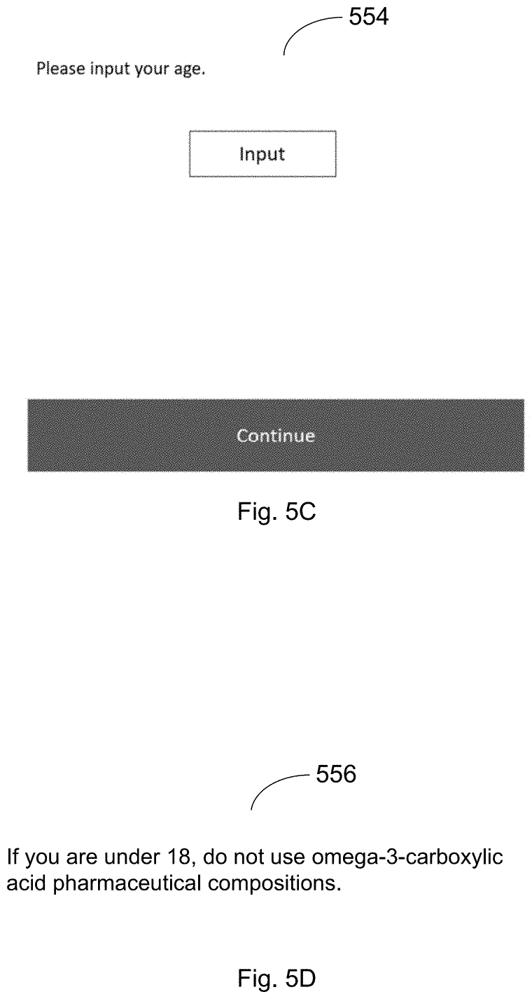

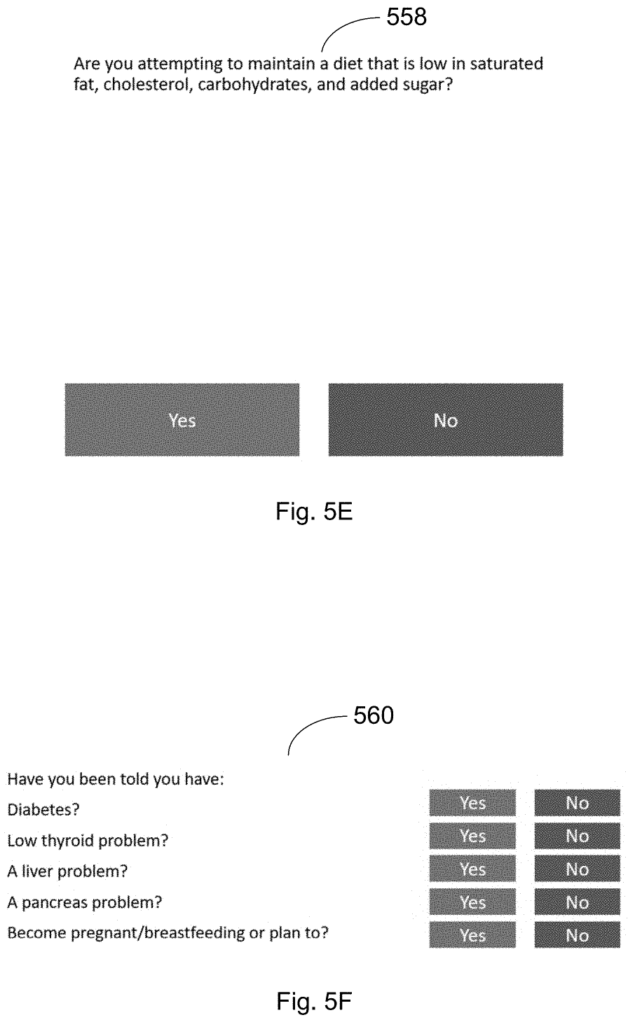

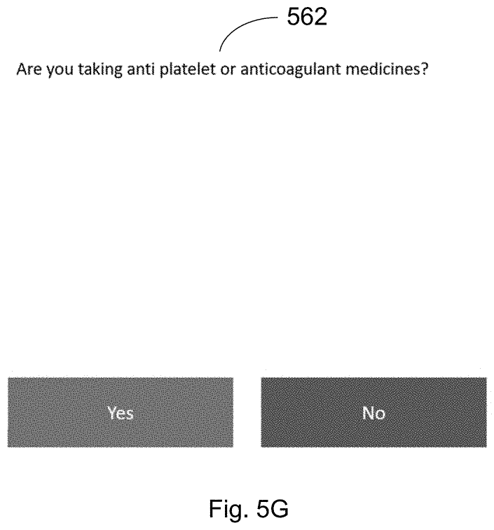

Referring to block 410, e.g., as illustrated in FIG. 7, in some embodiments the first survey results indicate a triglyceride level of the subject (e.g., responsive to a survey question 208, such as question 550 illustrated in FIG. 5A, e.g., that is associated with and/or applied to (706,708) a triglyceride level filter 216-1 of a first category class 214-1), an age of the subject (e.g., responsive to a survey question 208 that is associated with and/or applied to (710) an age filter 216-2 of a first category class), a dietary status of the subject (e.g., responsive to a survey question 558 that is associated with and/or applied to (712) a dietary filter 216-3 of a first category class), a diabetes of the subject (e.g., responsive to a survey question 212 that is associated with and/or applied to (714) a diabetes filter 222-1 of a second category class 220-1), a thyroid status of the subject (e.g., responsive to a survey question 212 that is associated with and/or applied to (716) a thyroid disease filter 222-2 of a second category class), whether the subject has a liver problem (e.g., responsive to a survey question 212 that is associated with and/or applied to (718) a liver disease filter 222-3 of a second category class), whether the subject has a pancreatic problem (e.g., responsive to a survey question 212 that is associated with and/or applied to (720) a pancreatic disease filter 222-4 of a second category class), whether the subject is one of pregnant, breastfeeding, or planning to become pregnant (e.g., responsive to a survey question 212 that is associated with and/or applied to (722) a pregnancy filter 222-5 of a second category class), whether the subject has a seafood allergy (e.g., responsive to a survey question 208 that is associated with and/or applied to (732) a first allergy filter 222-6 of a second category class), and whether the subject is taking a medication that affects coagulation (e.g., responsive to a survey question 55 that is associated with and/or applied to (724) a coagulant medication filter 222-7 of a second category class).

In some embodiments, the first survey includes questions that elicit responses providing or indicating some or all of the characteristics listed in Table 1. In some embodiments, the survey includes questions corresponding to each of the survey results required for the methods described herein. In other embodiments, the survey includes questions corresponding to only a subset of the survey results required for the methods described herein. In some embodiments, other survey results required for the methods described herein are acquired through other means (e.g., upon registration/subscription for a service associated with qualifying the subject for over-the-counter medication, from a healthcare provider, from a prior survey, from a database associated with a pharmacy, from an electronic health record associated with the subject, from the subject profile data store 232, etc.) For example, in some embodiments, the subject provides a personal medical identification associated with an insurer, a hospital, or other healthcare provider and information about the subject required for the methods described herein, e.g., one or more survey results, is acquired from a preexisting database associated with the personal medical identification (e.g., a last cholesterol or triglyceride level measurement determined for the subject).

TABLE 1
Example subject characteristics for qualifying a subject
for an over-the-counter provision of a concentrated
fish oil-based pharmaceutical composition

| Result | Example Characteristics |
|---|---|
| 1 | a triglyceride level of the subject |
| 2 | an age of the subject |
| 3 | a dietary status of the subject |
| 4 | a diabetes status of the subject |
| 5 | a thyroid status of the subject |
| 6 | whether the subject has a liver problem |
| 7 | whether the subject has a pancreatic problem |
| 8 | whether the subject is one of pregnant, breastfeeding, or |
| | planning to become pregnant |
| 9 | whether the subject has a seafood allergy |
| 10 | whether the subject is taking a medication that affects |
| | coagulation |
| 11 | whether the subject is allergic to the concentrated fish oil-based |
| | pharmaceutical composition |

It is contemplated that, in some embodiments, any one or more of the survey questions 208, 212 provided in Table 1 will not be included in the first survey (e.g., will not be used for the assessment). For example, in some embodiments, a characteristic associated with a particular survey questions will be informative when qualifying a subject for one particular concentrated fish oil but not for another concentrated fish oil.

Accordingly, it is contemplated that the first survey questions 208 include any subset of survey results provided in Table 1. For brevity, all possible combinations of survey questions 208, 212 eliciting the characteristics provided in Table 1 are not specifically delineated here. However, the skilled artisan will be able to envision any particular subset of the survey questions 208, 212 that elicit the characteristics provided in Table 1. Likewise, the skilled artisan may know of other survey questions, eliciting informative subject characteristics not provided in Table 1, that may be combined with any subset of survey questions that elicit subject characteristics provided in Table 1 to form the first survey questions used in the methods described herein.

In some embodiments, the first and/or second survey is conducted by transmitting a plurality of questions to the subject, e.g., some or all of the survey questions, and receiving answers to the plurality of survey questions before applying any of the answers to respective filters. For example, with reference to the workflow in FIG. 7, the device transmits questions relating to all of the filters of the first category class, all of the filters of the second category class, or all of the filters in the workflow (e.g., as a virtual survey where all of the questions are displayed in a single user interface, or as a series of questions displayed in consecutive user interfaces). After receiving answers to all of the survey questions, the device then applies the answers to all of the filters (e.g., sequentially or concurrently) to determine whether the subject is qualified to receive provision of the concentrated fish oil-based pharmaceutical composition. In alternative embodiments, the device transmits questions relating to just those filters of the first category class for which it could not obtain answers to the questions from an electronic database associated with the subject, such as electronic health record of the subject, and just those filters of the second category class it could not obtain answers to the questions from an electronic database associated with the subject (e.g., as a virtual survey where such unanswered questions are displayed in a single user interface, or as a series of questions displayed in consecutive user interfaces). After receiving answers to all of the survey questions, the device then applies the answers to all of the filters e.g., sequentially or concurrently) to determine whether the subject is qualified to receive provision of the concentrated fish oil-based pharmaceutical composition.

In some embodiments, the first and/or second survey is conducted in a serial fashion, e.g., by transmitting a first question or a first group of survey questions (e.g., associated with a single filter) to the subject, receiving an answer to the single survey question or small group of survey questions, and applying the answer or answers to a filter, prior to transmitting a second question or second group of questions to the subject. For example, with reference to the workflow in FIG. 7, in some embodiments the device transmits a first question to the subject, relating to the triglyceride level of the subject (e.g., question 550 ‘Please input your triglyceride levels.’ in FIG. 5A). After receiving the answer to the survey question (e.g., ‘a triglyceride level of 150 milligrams per deciliter (mg/dL)’), the device applies the answer to a first triglyceride level filter (706,708). If the first triglyceride level filter is fired (e.g., in response to an answer that correlates to a triglyceride level below 500 mg/dL or above 1000 mg/dL), the device terminates (705) the process, and optionally provides the user with a message relating to why they are being denied a provision of the concentrated fish oil-based pharmaceutical composition (e.g., as illustrated in FIG. 5D, message 556, advising the subject that omega-3-carboxylic acid pharmaceutical compositions are not for use if the subject is under 18), a suggestion for following-up with a medical professional (e.g., as illustrated in FIG. 7A as well as FIG. 5F, message 560, when the survey answers indicate that the subject is in hypertriglyceridemia (708), the device terminates the process (705) and advises that the subject seek medical attention and/or consult their licensed medical practitioner), and/or a suggestion to make a lifestyle change (e.g., as illustrated in FIG. 7A, when the survey answers indicate that the subject's diet is not appropriate (712), the device terminates the process (707) and advises that the subject improve their diet or exercise routine), to treat or manage their triglyceride level.

Blocks 412-428. Referring to block 412 of FIG. 4B, all or a portion of the first survey results are run against a first plurality of filters of a first category class 214. As previously described, the first plurality of filters includes a subset of filters 216 of the first filter category class 214. When a respective filter in the first plurality of filters is fired (e.g., when a survey result indicates that a triggering condition 218 has been met), the subject is deemed not qualified for delivery of the concentrated fish oil-based pharmaceutical composition and the method is terminated without delivery of the concentrated fish oil-based pharmaceutical composition.

In some embodiments, when the method is terminated without delivery of the concentrated fish oil-based pharmaceutical composition, the subject is prevented from attempting to requalify for the concentrated fish oil-based pharmaceutical composition for a predetermined period of time. This prevents the subject from abusing the systems and methods of the present disclosure.

Referring to blocks 414-428, specific filters 216 in the first plurality of filters and their exemplary triggering conditions 218 that cause the corresponding filter to fire are detailed.

In some embodiments, the first plurality of filters of the first category class 214 includes some or all of the filters 216 listed in Table 2. For example, in some embodiments, the first plurality of filters results includes 2, 3, or all 4 of the filters listed in Table 2.

TABLE 2
Example filters for contraindications associated with qualifying
a subject for an over-the-counter provision of a concentrated
fish oil-based pharmaceutical composition

| Filter | Example Criteria |
|---|---|
| 1a | a triglyceride level filter |
| 2a | an age filter |
| 3a | a dietary filter |
| 4a | a fish oil allergy filter |

In one embodiment, the first plurality of filters includes at least filters 1a-4a as provided in Table 2. In another embodiment, the first plurality of filters includes at least filters 1a, 2a, and 3a as provided in Table 2. In another embodiment, the first plurality of filters includes at least filters 1a, 2a, and 4a as provided in Table 2. In another embodiment, the first plurality of filters includes at least filters 1a, 3a, and 4a, as provided in Table 2. In another embodiment, the first plurality of filters includes at least filters 2a, 3a, and 4a as provided in Table 2. In another embodiment, the first plurality of filters includes at least filters 1a and 2a as provided in Table 2. In another embodiment, the first plurality of filters includes at least filters 1a and 3a as provided in Table 2. In another embodiment, the first plurality of filters includes at least filters 1a and 4a as provided in Table 2. In another embodiment, the first plurality of filters includes at least filters 2a and 3a as provided in Table 2. In another embodiment, the first plurality of filters includes at least filters 2a and 4a as provided in Table 2. In another embodiment, the first plurality of filters includes at least filters 3a and 4a as provided in Table 2.

It is contemplated that, in some embodiments, any one or more of the filters 216 provided in Table 2 will not be included in the first plurality of filters. For example, in some embodiments, a characteristic associated with a particular survey result will be informative when qualifying a subject for one particular concentrated fish oil but not for another concentrated fish oil.

Accordingly, it is contemplated that the first plurality of filters includes any sub-set of filters 216 provided in Table 2. Likewise, the skilled artisan may know of other filters 216, not provided in Table 2, which may be combined with any subset of the filters 216 provided in Table 2 to form the first plurality of filters results used in the methods described herein. For brevity, all possible combinations of the filters 216 provided in Table 2 are not specifically delineated here.

Referring to blocks 414-418, in some embodiments the first plurality of filters includes a triglyceride filter (e.g., triglyceride filter 216-1 in FIG. 3 and/or filter 1a in Table 2). In some embodiments, the triglyceride filter is configured to be fired at least when the first plurality of survey results indicates that the subject has a triglyceride level that is either below a floor triglyceride level or above a ceiling triglyceride level. In some embodiments, the floor triglyceride level used in the first triglyceride level filter is 500 mg/dL. In some embodiments, the ceiling triglyceride level used in the first triglyceride level filter is 1000 mg/dL. In some embodiments, the floor triglyceride level is set lower, to enable more subjects over-the-counter access to the concentrated fish oil pharmaceutical composition. In some embodiments, the floor triglyceride level is set at from 150 to 500 mg/dL. In one embodiment, the floor triglyceride level is set at 180 mg/dL. In some embodiments, the ceiling triglyceride level and the ceiling triglyceride level that are used in the first triglyceride level filter are set according to a set of healthcare guidelines, which may change over time, and/or vary on a jurisdiction-by-jurisdiction basis. For example, in the United States, the American Heart Association and the American College of Endocrinology collaborated to provide guidance on management of dyslipidemia (e.g., hyperlipidemia) and prevention of cardiovascular disease. Jellinger, et al., Endocrine Practice., 23(2), (2017), the contents of which are hereby expressly incorporated by reference. These guidelines change over time as medical research and advances in treatment better inform management of triglyceride levels.

When the triglyceride filter is fired, the subject is not permitted to obtain the concentrated fish oil-based pharmaceutical composition over-the-counter (e.g., the method is terminated without authorizing provision of the concentrated fish oil-based pharmaceutical composition to the subject). For example, the device transmits prompt 550, as illustrated in FIG. 5A, to the subject and the device applies the subject's answer to the triglyceride filter (e.g., triglyceride filter 705 in FIG. 7A). If the subject's answer indicates that they are outside of a predetermined triglyceride level range (e.g., the triglyceride level of the subject is less than 500 mg/dL or greater than 1000 mg/dL, or is less than or equal to 500 mg/dL or greater than or equal to 1000 mg/dL), the triglyceride level filter is fired, and the method is terminated without authorizing provision of the concentrated fish oil-based pharmaceutical composition to the subject. In some embodiments, the device transmits a message explaining why authorization was denied, e.g., message 552 illustrated in FIG. 5B.

Referring to blocks 420-422, in some embodiments the first plurality of filters includes an age filter (e.g., age filter 216-2 in FIG. 3 and/or filter 2a in Table 2). In some embodiments, the age filter is fired when the first plurality of survey results indicates that the subject has not yet reach an age of majority e.g., is less than eighteen years old (e.g., as illustrated at 710 in FIG. 7A). If the age filter is fired, the subject is not permitted to obtain the concentrated fish oil-based pharmaceutical composition pharmaceutical composition over-the-counter (e.g., the method is terminated without authorizing provision of the concentrated fish oil-based pharmaceutical composition to the subject).

Referring to blocks 424-426, in some embodiments the first plurality of filters includes a dietary filter (e.g., dietary filter 216-3 in FIG. 3 and/or filter 3a in Table 2). The dietary pressure filter is configured to be fired at least when the first plurality of survey results indicates that the subject indicates that the subject's diet is not appropriate, e.g., according to current health guidelines. In some embodiments, an inappropriate diet is one that is not low in saturated fat, low in cholesterol, low in carbohydrates, and/or low in added sugar. If the dietary filter is fired, the subject is not permitted to obtain the concentrated fish oil-based pharmaceutical composition over-the-counter (e.g., the method is terminated without authorizing provision of the concentrated fish oil-based pharmaceutical composition to the subject).

Referring to block 428, in some embodiments the first plurality of survey results further includes whether the subject is allergic to the concentrated fish oil-based pharmaceutical composition, and the first plurality of filters includes an adverse reaction filter. The adverse reaction filter is fired when the first survey results indicate that the subject is allergic to the concentrated fish oil-based pharmaceutical composition. In some embodiments, the adverse reaction filter is fired when the first survey results indicate that the subject has developed an adverse reaction to a concentrated fish oil-based medication in the past. In some embodiments, the adverse reaction filter is fired when the first survey results indicate that the subject has developed an adverse reaction to any concentrated fish oil-based pharmaceutical composition in the past.

Referring to block 430 of FIG. 4C, the method also includes running all or a portion of the first survey results against a second plurality of filters of a second category class 220. When a respective filter in the second plurality of filters is fired, the subject is provided with a warning 226 corresponding to the respective filter (e.g., filter warning 226-4 corresponds to filter 222-4). In some embodiments, the warning 226 is provided as a next step, e.g., prior to applying survey results to any subsequent filters, after the corresponding filter is fired. For example, with respect to FIG. 7B, in some embodiments, when the liver disease filter is triggered at 718, the device would provide the subject with a warning prior to proceeding to the pancreatic disease filter at 720, e.g., requiring the subject confirm they have discussed their history of liver disease with a health care provider, e.g., and the healthcare provider still recommends taking a concentrated fish oil-based pharmaceutical composition in order to proceed with the qualification. In some embodiments the warning 226 is provided after applying survey results to all subsequent filters. For example, as illustrated in FIG. 7B, in some embodiments, when the liver disease filter is triggered at 718, the device would proceed to the pancreatic disease filter at 720 prior to transmitting a warning to the subject, and then transmit all warnings corresponding to filters of the second category class, at 726, after survey results have been applied to all subsequent filters.

In some embodiments, the second plurality of filters 222 of the second category class 220 includes some or all of the filters listed in Table 3. For example, in some embodiments, the first plurality of filters results includes 2, 3, 4, 5, 6, or all 7 of the filters listed in Table 3. In one embodiment, the first plurality of filters includes all of filters 1-7 as provided in Table 3.

TABLE 3
Example filters for risk factors associated with qualifying
a subject for an over-the-counter provision of a
concentrated fish-oil based pharmaceutical composition

| Filter | Example Criteria |
|---|---|
| 1a | a diabetes filter |
| 2a | a thyroid disease filter |
| 3a | a liver disease filter |
| 4a | a pancreatic disease filter |
| 5a | a pregnancy filter |
| 6a | an allergy filter |
| 7a | a coagulant medication filter |

Referring to block 432, in some embodiments, the second plurality of filters includes a diabetes filter (e.g., diabetes filter 222-1 in FIG. 3 and/or filter 1a in Table 3). The diabetes filter is configured to be fired at least when the first plurality of survey results indicate that the subject has diabetes (e.g., the subject has Type 1 diabetes, the subject has Type 2 diabetes, etc.) When the diabetes filter is fired, the device transmits a warning corresponding to the diabetes filter, and requires the user to acknowledge the warning before authorizing a provision of the concentrated fish oil-based pharmaceutical composition.

Referring to block 434, in some embodiments, the second plurality of filters includes a thyroid disease filter (e.g., thyroid disease filter 222-2 in FIG. 3 and/or filter 2a in Table 3). The thyroid disease filter is configured to be fired at least when the first plurality of survey results indicates that the subject has a thyroid problem (e.g., hypothyroidism). When the thyroid disease filter is fired, the device transmits a warning corresponding to the thyroid disease filter, and requires the user to acknowledge the warning before authorizing a provision of the concentrated fish oil-based pharmaceutical composition.

Referring to block 436, in some embodiments, the second plurality of filters includes a liver disease filter (e.g., liver disease filter 222-3 in FIG. 3 and/or filter 3a in Table 3). The liver disease filter is configured to be fired at least when the first plurality of survey results indicates that the subject has a liver problem. In some embodiments, liver problems that are capable of triggering the first liver disease filter include impaired hepatic function, acute liver failure, and cholestasis. When the liver disease filter is fired, the device transmits a warning corresponding to the liver disease filter, and requires the user to acknowledge the warning before authorizing a provision of the concentrated fish oil-based pharmaceutical composition.

Referring to block 438, in some embodiments, the second plurality of filters includes a pancreatic filter (e.g., pancreatic disease filter 222-4 in FIG. 3 and/or filter 4a in Table 3). The pancreatic disease filter is configured to be fired at least when the first plurality of survey results indicates that the subject has a pancreatic problem (e.g., pancreatitis). When the pancreatic disease filter is fired, the device transmits a warning corresponding to the pancreatic disease filter, and requires the user to acknowledge the warning before authorizing a provision of the concentrated fish oil-based pharmaceutical composition.

Referring to blocks 440 and 442, in some embodiments, the second plurality of filters includes a pregnancy filter (e.g., pregnancy filter 222-5 in FIG. 3 and/or filter 5a in Table 3). In some embodiments, the pregnancy filter is configured to be fired at least when the first plurality of survey results indicates that the subject is pregnant or the subject is breastfeeding. In some embodiments, the pregnancy filter is also configured to be fired when the subject is planning on becoming pregnant. When the pregnancy filter is fired, the subject is not permitted to obtain the concentrated fish oil-based pharmaceutical composition over-the-counter (e.g., the method is terminated without authorizing provision of the fish oil-based pharmaceutical composition to the subject). For example, the device transmits prompt 560, as illustrated in FIG. 5F, to the subject and the device applies the subject's answer to the pregnancy filter. If the subject's answer indicates that they are pregnant, they are planning on being pregnant, they are breastfeeding, or they are planning to breastfeeding, the pregnancy filter is fired, and the method is terminated without authorizing provision of the fish oil-based pharmaceutical composition to the subject. In some embodiments, the device transmits a message explaining why authorization was denied.

Referring to block 444, in some embodiments, the second plurality of filters includes an allergy filter (e.g., allergy filter 222-6 in FIG. 3 and/or filter 6a in Table 3). This allergy filter is configured to be fired at least when the first plurality of survey results indicates that the subject is allergic to fish or shellfish (e.g., mollusks and crustaceans). In some embodiments, the allergy filter is configured to be fired at least when the first plurality of survey results indicates that the subject is allergic to seafood. When this allergy filter is fired, the device transmits a warning corresponding to the first allergy filter, and requires the user to acknowledge the warning before authorizing a provision of the concentrated fish oil-based pharmaceutical composition.

Referring to block 446, in some embodiments, the second plurality of filters includes a coagulant medication filter (e.g., coagulant medication filter 222-7 in FIG. 3 and/or filter 7a in Table 3). The coagulant medication filter is configured to be fired at least when the first plurality of survey results indicates that the subject is taking an anti-platelet medication or an anti-coagulant medication. When the coagulant medication filter is fired, the device transmits a warning corresponding to the coagulant medication filter, and requires the user to acknowledge the warning before authorizing a provision of the concentrated fish oil-based pharmaceutical composition.

It is contemplated that, in some embodiments, any one or more of the filters provided in Table 3 will not be included in the second plurality of filters. For example, in some embodiments, a characteristic associated with a particular survey result will be informative when qualifying a subject for one particular concentrated fish oil-based pharmaceutical composition but not for another concentrated fish oil-based pharmaceutical composition. Accordingly, it is contemplated that the second plurality of filters includes any sub-set of filters provided in Table 3. Likewise, the skilled artisan may know of other filters, not provided in Table 3, that may be combined with any subset of the filters provided in Table 3 to form the second plurality of filters results used in the methods described herein.

Contraindications and risk factors described in the present disclosure are non-exhaustive. The skilled artisan may know of other contraindications for a particular the concentrated fish oil-based pharmaceutical composition and/or treat risk factors as contraindications dependent upon the intended use of the concentrated fish oil-based pharmaceutical composition. In some embodiments, contraindications for use of a prescription-strength pharmaceutical agent are treated only as risk factors, or not at all, when qualifying a subject for a lower-dose OTC use of a concentrated fish oil-based pharmaceutical composition.

Accordingly, it will be appreciated that the survey questions 208, 212, and filters 216, 222 applied to the survey answers thereof, may vary depending upon the concentrated fish oil-based pharmaceutical composition being distributed. This is due to differences in the contraindication profiles of the various the concentrated fish oil-based pharmaceutical compositions, e.g., due to different drug-drug interactions, routes of drug clearance, etc. of the different the concentrated fish oil-based pharmaceutical compositions.

Referring to block 450 of FIG. 4D, the method includes obtaining acknowledgment from the subject for any warning 226 issued to the subject by any filter 222 in the second plurality of filters. In some embodiments, acknowledgment from the subject is a written acknowledgement, a verbal acknowledgment, or an electronic acknowledgment such as an electronic signature. If a filter 216 in the first plurality of filters fires, the subject is denied access to the over-the-counter concentrated fish oil-based pharmaceutical composition.

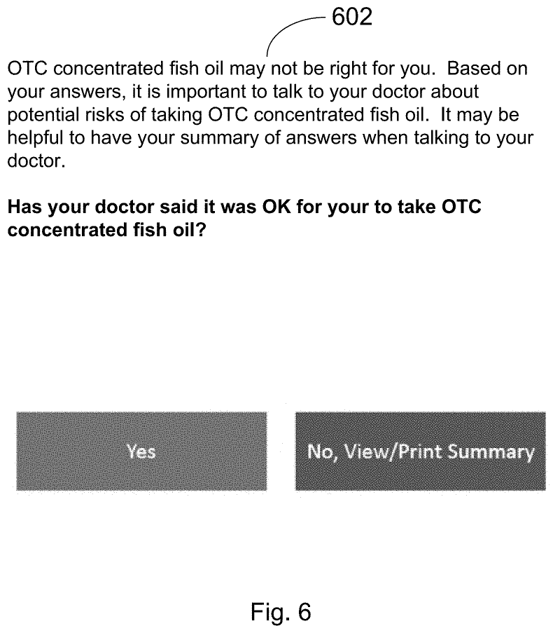

Referring to block 448, in some embodiments the warning 226 corresponding to a respective filter 222 in the second plurality of filters includes a prompt for the subject to indicate whether they have discussed the risk factor underlying the respective filter in the second plurality of filters that was fired with a health care practitioner (e.g., a licensed medical practitioner), e.g., and the health care practitioner indicated that the subject should take a concentrated fish oil-based pharmaceutical composition in view of the underlying risk factor. Accordingly, acknowledgement is obtained when the subject indicates that they have discussed the risk factor underlying the respective filter in the second plurality of filters that was fired with a health care provider. For example, message 602 in FIG. 6 illustrates an example warning that is generic to any fired filters. In some embodiments, the warning is specific to a particular filter (e.g., filter warning 226 in FIG. 2), e.g., communicating to the user why the filter was fired.

In some embodiments, an acknowledgment from the user is verified by the health care practitioner (e.g., the method requires verification in order for authorization of the provision of the concentrated fish oil-based pharmaceutical composition), e.g., in order to verify an accuracy of the survey results of the subject. In some embodiments, when the acknowledgment is verified by the heath care practitioner, the subject is deemed a trusted subject, such that verification of future results is not required.

Blocks 452-462. Referring to block 452, the process control proceeds to the fulfillment process when no filter 216 in the first plurality of filters has been fired and the subject has acknowledged each warning 226 associated with each filter 222 in the second plurality of filters that was fired. In some embodiments, the fulfillment process includes storing an indication in a user profile 234 of an initial order date and/or destination for the concentrated fish oil-based pharmaceutical composition. In some embodiments, the initial order date is utilized, for example, to verify at least a refill status of a provision of the concentrated fish oil. In some embodiments, the initial order date is utilized, for example, to verify at least an elapsed period of time between an initial order and a future re-order. Such verification is required in order to ensure that certain tests (e.g., triglyceride level tests) are taken regularly.

The fulfillment process further includes communicating an over-the-counter drug facts label 230 for the concentrated fish oil-based pharmaceutical composition to the subject. In some embodiments, the drug facts label is communicated to the subject in real-time, e.g., within the same user interface as used for the qualification process. In some embodiments, the over-the-counter drug facts label 230 specifies what the concentrated fish oil-based pharmaceutical composition is for (e.g., for lowering triglyceride levels, e.g., thereby treating or preventing hypertriglyceridemia and/or heart disease etc.), what dosage the subject is being authorized to take, and/or any risks associated with taking the concentrated fish oil-based pharmaceutical composition (e.g., drug-drug interactions, pharmacokinetic interactions, adverse reactions, etc.). For instance, in some embodiments, upon confirmation from the subject that the over the counter drug facts label 230 has been received and read, the subject is authorized for provision of a dosage of from 0.5 g to 5 g of the concentrated fish oil-based pharmaceutical composition no more than once per day. In another example embodiment, upon confirmation from the subject that the over the counter drug facts label 230 has been received and read, the subject is authorized for provision of a dosage of from 0.5 g to 2 g of the concentrated fish oil-based pharmaceutical composition no more than once per day. In yet another example embodiment, upon confirmation from the subject that the over the counter drug facts label 230 has been received and read, the subject is authorized for provision of a dosage of 2 g of the concentrated fish oil-based pharmaceutical composition no more than once per day.

In some embodiments, upon confirmation from the subject that the over the counter drug facts label 230 has been received and read, the subject is authorized for provision of a dosage of from 0.5 g to 5 g of an omega-3-carboxylic acid pharmaceutical composition no more than once per day (block 456). In some embodiments, upon confirmation from the subject that the over the counter drug facts label 230 has been received and read, the subject is authorized for provision of a dosage of from 0.5 g to 4 g of an omega-3-carboxylic acid pharmaceutical composition no more than once per day. In some embodiments, upon confirmation from the subject that the over the counter drug facts label 230 has been received and read, the subject is authorized for provision of a dosage of from 0.5 g to 2.5 g of an omega-3-carboxylic acid pharmaceutical composition no more than once per day. In some embodiments, upon confirmation from the subject that the over the counter drug facts label 230 has been received and read, the subject is authorized for provision of a dosage of from 0.5 g to 1 g of an omega-3-carboxylic acid pharmaceutical composition no more than once per day. In some embodiments, upon confirmation from the subject that the over the counter drug facts label 230 has been received and read, the subject is authorized for provision of a dosage of from 1 g to 2 g of an omega-3-carboxylic acid pharmaceutical composition no more than once per day. In some embodiments, upon confirmation from the subject that the over the counter drug facts label 230 has been received and read, the subject is authorized for provision of a dosage of 2 g of an omega-3-carboxylic acid pharmaceutical composition no more than once per day (block 458).

In some embodiments, upon confirmation from the subject that the over the counter drug facts label 230 has been received and read, the subject is authorized for provision of a dosage of from 0.5 g to 5 g of an omega-3 acid ethyl ester pharmaceutical composition no more than once per day. In some embodiments, upon confirmation from the subject that the over the counter drug facts label 230 has been received and read, the subject is authorized for provision of a dosage of from 0.5 g to 4 g of an omega-3 acid ethyl ester pharmaceutical composition no more than once per day. In some embodiments, upon confirmation from the subject that the over the counter drug facts label 230 has been received and read, the subject is authorized for provision of a dosage of from 0.5 g to 2.5 g of an omega-3 acid ethyl ester pharmaceutical composition no more than once per day. In some embodiments, upon confirmation from the subject that the over the counter drug facts label 230 has been received and read, the subject is authorized for provision of a dosage of from 0.5 g to 1 g of an omega-3 acid ethyl ester pharmaceutical composition no more than once per day. In some embodiments, upon confirmation from the subject that the over the counter drug facts label 230 has been received and read, the subject is authorized for provision of a dosage of from 1 g to 2 g of an omega-3 acid ethyl ester pharmaceutical composition no more than once per day. In some embodiments, upon confirmation from the subject that the over the counter drug facts label 230 has been received and read, the subject is authorized for provision of a dosage of 2 g of an omega-3 acid ethyl ester pharmaceutical composition no more than once per day.

In some embodiments, upon confirmation from the subject that the over the counter drug facts label 230 has been received and read, the subject is authorized for provision of a dosage of from 0.5 g to 5 g of an ethyl eicosapentaenoic acid pharmaceutical composition no more than once per day. In some embodiments, upon confirmation from the subject that the over the counter drug facts label 230 has been received and read, the subject is authorized for provision of a dosage of from 0.5 g to 4 g of an ethyl eicosapentaenoic acid pharmaceutical composition no more than once per day. In some embodiments, upon confirmation from the subject that the over the counter drug facts label 230 has been received and read, the subject is authorized for provision of a dosage of from 0.5 g to 2.5 g of an ethyl eicosapentaenoic acid pharmaceutical composition no more than once per day. In some embodiments, upon confirmation from the subject that the over the counter drug facts label 230 has been received and read, the subject is authorized for provision of a dosage of from 0.5 g to 1 g of an ethyl eicosapentaenoic acid pharmaceutical composition no more than once per day. In some embodiments, upon confirmation from the subject that the over the counter drug facts label 230 has been received and read, the subject is authorized for provision of a dosage of from 1 g to 2 g of an ethyl eicosapentaenoic acid pharmaceutical composition no more than once per day. In some embodiments, upon confirmation from the subject that the over the counter drug facts label 230 has been received and read, the subject is authorized for provision of a dosage of 2 g of an ethyl eicosapentaenoic acid pharmaceutical composition no more than once per day.

In some embodiments, upon confirmation from the subject that the over the counter drug facts label 230 has been received and read, the subject is authorized for provision of a dosage of from 0.5 g to 5 g of a krill oil pharmaceutical composition no more than once per day. In some embodiments, upon confirmation from the subject that the over the counter drug facts label 230 has been received and read, the subject is authorized for provision of a dosage of from 0.5 g to 4 g of a krill oil pharmaceutical composition no more than once per day. In some embodiments, upon confirmation from the subject that the over the counter drug facts label 230 has been received and read, the subject is authorized for provision of a dosage of from 0.5 g to 2.5 g of a krill oil pharmaceutical composition no more than once per day. In some embodiments, upon confirmation from the subject that the over the counter drug facts label 230 has been received and read, the subject is authorized for provision of a dosage of from 0.5 g to 1 g of a krill oil pharmaceutical composition no more than once per day. In some embodiments, upon confirmation from the subject that the over the counter drug facts label 230 has been received and read, the subject is authorized for provision of a dosage of from 1 g to 2 g of a krill oil pharmaceutical composition no more than once per day. In some embodiments, upon confirmation from the subject that the over the counter drug facts label 230 has been received and read, the subject is authorized for provision of a dosage of 2 g a krill oil pharmaceutical composition no more than once per day.

Referring to block 460, in some embodiments the fulfillment process further includes authorizing provision of the concentrated fish oil-based pharmaceutical composition to the subject. The authorization occurs upon confirmation from the subject that the over-the-counter drug facts label 230 has been received and read by the subject. In some embodiments, this authorization includes a destination associated with the subject. In some embodiments, the destination associated with the subject is stored in the user profile 234. In some embodiments, the destination associated with the subject is a physical address including a street address, a Post Office box, a pharmacy associated with the subject, a health care provider associated with the subject, and/or one or more coordinates (e.g., longitude, latitude, elevation). In some embodiments, the provision of the concentrated fish oil-based pharmaceutical composition to the subject comprises shipping the concentrated fish oil-based pharmaceutical composition to the physical address associated with the subject (block 462). In some embodiments, the provision of the concentrated fish oil-based pharmaceutical composition to the subject comprises shipping the concentrated fish oil-based pharmaceutical composition to a pharmacy associated and/or a location associated with a health care provider of the subject and/or an office of a medical practitioner associated with the subject.

Blocks 464-494. Referring to blocks 464-494 of FIGS. 4E-4G, a re-fulfillment process will be described infra. In some embodiments, the present disclosure provides a method for qualifying a subject for a refill of a concentrated fish oil-based pharmaceutical composition. In some embodiments, the qualification for a refill of the concentrated fish oil-based pharmaceutical composition follows an initial qualification of the subject, as described herein. In some embodiments, the qualification for a refill of the concentrated fish oil-based pharmaceutical composition follows issuance of a prescription to the subject for the concentrated fish oil-based pharmaceutical composition. For example, in some embodiments, a subject who is new to the qualification process is asked whether they previously received a prescription for the concentrated fish oil-based pharmaceutical composition and, if the subject indicates that they have not previously received a prescription, the subject is directed to an initial qualification method and, if the subject indicates that they have previously received a prescription, the subject is directed to the refill qualification method, e.g., as described below.

Referring to block 464 of FIG. 4E, in some embodiments a re-fulfillment procedure is performed. The re-fulfillment procedure is responsive to receiving a re-order request from the subject for the concentrated fish oil-based pharmaceutical composition. In some embodiments, a prompt to initiate the re-fulfillment procedure is sent to user device 102 associated with the subject after a predetermined amount of time associated with a duration of dosages previously delivered to the subject (e.g., the user is reminded to fulfill their order of the concentrated fish oil-based pharmaceutical composition just before, or just after, the user is scheduled to run out of a previously delivered provision.

Referring to blocks 466-468, in some embodiments the re-fulfillment procedure includes conducting a second survey of the subject. The second survey is configured to obtain a second plurality of survey results. These results are derived from corresponding survey questions (e.g., the device transmits one or more survey questions to the user, prompting a response, and then receives a response to the one or more survey questions back from the subject). In some embodiments, the second plurality of survey results include, or at least indicate, some or all of the subject characteristics listed in Table 4. For example, in some embodiments, the second plurality of survey results includes, or at least indicates, 1, 2, 3, 4, 5, 6, 7, 8, 9, or all 10 of the characteristic listed in Table 4. In one embodiment, the second survey questions and results include at least characteristics 1-9 as provided in Table 4.

In some embodiments, the second survey results comprises at least one of: a dietary status of the subject (e.g., responsive to a survey question that is associated with and/or applied to (806) a dietary filter 216-4 of a first category class 214-2), if a predetermined period of time has passed since the subject took a previous blood, a triglyceride level of the subject (e.g., responsive to a survey question that is associated with and/or applied to (808,810) a triglyceride filter 216-5 of a first category class), a diabetes status of the subject (e.g., responsive to a survey question that is associated with and/or applied to (812) a diabetes filter 222-8 of a second category class 220-2), a thyroid status of the subject (e.g., responsive to a survey question that is associated with and/or applied to (814) a thyroid disease filter 222-9 of a second category class), a liver function status of the subject (e.g., responsive to a survey question that is associated with and/or applied to (816) a liver disease filter 222-10 of a second category class), a pancreatic function status of the subject (e.g., responsive to a survey question that is associated with and/or applied to (818) a pancreatic disease filter 222-11 of a second category class), whether the subject is one of pregnant, breastfeeding, or planning to become pregnant (e.g., responsive to a survey question that is associated with and/or applied to (820) a pregnancy filter 222-12 of a second category class), and whether the subject is taking a medication that affects coagulation (e.g., responsive to a survey question that is associated with and/or applied to (828) a coagulant medication filter 222-13 of a second category class).

In some embodiments, the second survey includes questions that elicit responses providing some or all of the characteristics listed in Table 4. In some embodiments, the second survey includes questions corresponding to each of the survey results required for the methods described herein. In other embodiments, the second survey includes questions corresponding to only a subset of the survey results required for the methods described herein. In some embodiments, other survey results required for the methods described herein are acquired through other means (e.g., upon registration/subscription for a service associated with qualifying the subject for over-the-counter medication, from a healthcare provider, from a prior survey, from a database associated with a pharmacy, etc.) For example, in some embodiments, the subject provides a personal medical identification associated with an insurer, a hospital, or other healthcare provider and information about the subject required for the methods described herein, e.g., one or more survey results, is acquired from a preexisting database associated with the personal medical identification (e.g., a last liver function test or triglyceride measurement determined for the subject).

TABLE 4
Example characteristics for re-qualifying a subject for an over-the-counter
provision of a concentrated fish oil-based pharmaceutical composition

| Result | Example Characteristics |
|---|---|
| 1 | a dietary status of the subject, |
| 2 | if a predetermined period of time has passed since the user |
| | took a previous blood test |
| 3 | a triglyceride level status of the subject |
| 4 | a diabetes status of the subject |
| 5 | a thyroid status of the subject |
| 6 | a liver function status of the subject |
| 7 | a pancreatic function status of the subject |
| 8 | whether the subject is one of pregnant, breastfeeding, or |
| | planning to become pregnant |
| 9 | whether the subject is taking a medication that affects |
| | coagulation |
| 10 | whether the subject has developed a side effect from the |
| | concentrated fish oil-based pharmaceutical composition |

It is contemplated that, in some embodiments, any one or more of the survey questions provided in Table 4 will not be included in the second survey (e.g., will not be used for the reassessment). For example, in some embodiments, a characteristic associated with a particular survey question will be informative when qualifying a subject for one particular concentrated fish oil but not for another concentrated fish oil. For instance, in some embodiments a survey question is queried for omega-3-carboxylic acid qualifying surveys but not for krill oil qualifying surveys. The skilled artisan will recognize that different concentrated fish oils carry different risk and drug interaction profiles. Accordingly, survey information required for qualifying a subject for access to one concentrated fish oil with a known adverse drug interaction may not be necessary for qualifying the same subject for access to a second concentrated fish oil.

Accordingly, it is contemplated that the second survey questions elicit responses to any sub-set of survey results provided in Table 4. For brevity, all possible combinations of the characteristics provided in Table 4 are not specifically delineated here. However, the skilled artisan will easily be able to envision any particular subset of survey questions designed to elicit responses to any subset of characteristics provided in Table 4. Likewise, the skilled artisan may know of other survey questions, not provided in Table 4, that may be combined with any subset of the survey questions provided in Table 4 to form the second survey questions used in the methods described herein.

Referring to block 470, all or a portion of the results are run against a third plurality of filters of the first category class. When a respective filter in the third plurality of filters is fired (e.g., when a survey result indicates that a triggering condition 218 has been met), the subject is deemed not qualified for the concentrated fish oil-based pharmaceutical composition and the method is terminated without delivery of the concentrated fish oil-based pharmaceutical composition.

Referring to blocks 472-478, specific filters in the third plurality of filters and their exemplary triggering conditions that cause the corresponding filter to fire are detailed.

In some embodiments, the third plurality of filters of the first category class include some or all of the filters listed in Table 5. For example, in some embodiments, the third plurality of filters includes one or both of the filters listed in Table 5.

TABLE 5
Example filters for contraindications associated with re-qualification
of a subject for an over-the-counter provision of a concentrated
fish oil-based pharmaceutical composition

| Filter | Example Criteria |
|---|---|
| 1a | a dietary filter |
| 2a | a triglyceride level filter |

In one embodiment, the third plurality of filters includes at least filters 1a-2a as provided in Table 5. In another embodiment, the third plurality of filters includes at least filter 1a as provided in Table 5. In another embodiment, the first plurality of filters includes at least filter 2a as provided in Table 5.

It is contemplated that, in some embodiments, any one or more of the filters provided in Table 5 will not be included in the third plurality of filters. For example, in some embodiments, a characteristic associated with a particular survey result will be informative when qualifying a subject for one particular concentrated fish oil but not for another concentrated fish oil. Likewise, the skilled artisan may know of other filters, not provided in Table 5, which may be combined with any subset of the filters provided in Table 2 to form the third plurality of filters results used in the methods described herein. For brevity, all possible combinations of the filters provided in Table 5 are not specifically delineated here.

Referring to blocks 472 and 473, in some embodiments the third plurality of filters includes a dietary filter. In some embodiments, the dietary filter is as described above in relation to the first survey. In some embodiments, the dietary filter is configured to be fired at least when the first plurality of survey results indicates that the subject is not attempting to maintain an appropriate diet (e.g., a diet that is low in saturated fat, low in cholesterol, low in carbohydrates, and/or low in added sugar). When the dietary filter is fired, the subject is not permitted to obtain the concentrated fish oil-based pharmaceutical composition over-the-counter (e.g., the method is terminated without authorizing re-provision of the concentrated fish oil-based pharmaceutical composition to the subject). In some embodiments, the second plurality of survey results indicates that the subject is not attempting to maintain an appropriate diet when the survey results indicate that the subject has been diagnosed a poor diet since receiving their last provision of the concentrated fish oil-based pharmaceutical composition.

Referring to blocks 474-478, in some embodiments the third plurality of filters includes a triglyceride level filter. In some embodiments, the triglyceride level filter is as described above in relation to the first survey. In some embodiments, the triglyceride level filter is configured to be fired at least when the second plurality of survey results indicates that the subject is not maintaining an appropriate triglyceride level (e.g., the subject still has hypertriglyceridemia). In some embodiments, the second triglyceride filter is configured to be fired when a predetermined period of time has passed since the user took the last blood test indicated in the subject profile for the subject and the second plurality of survey results indicates that the subject has a triglyceride level that is above a maintenance triglyceride level (block 476). In some embodiments, the predetermined period of time is 12 months. In some embodiments, the predetermined period of time is 3 months, 6 months, 9 months, 15 months, or 18 months. In some embodiments, the maintenance triglyceride level used in the second triglyceride level filter is 500 mg/dL (block 478). In some embodiments, the maintenance triglyceride level used in the triglyceride level filter is 499 mg/dL or is 501 mg/dL.

In some embodiments, the device accounts for gaps in the subject's use of the concentrated fish oil-based pharmaceutical composition when determining whether the subject's triglyceride level is being effectively managed by administration of the composition (e.g., in some embodiments, where the device determines that the user has been without a provision of the concentrated fish oil-based pharmaceutical composition for a threshold period of time, the device bypasses the triglyceride level filter, or relaxes the requirements of the filter, for example, to a triglyceride level below that of a maintenance triglyceride level).

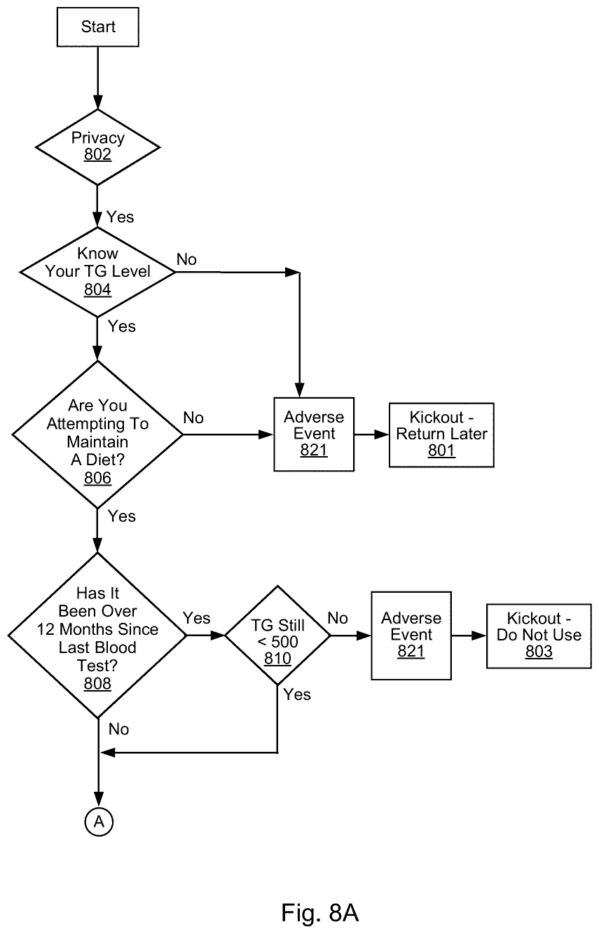

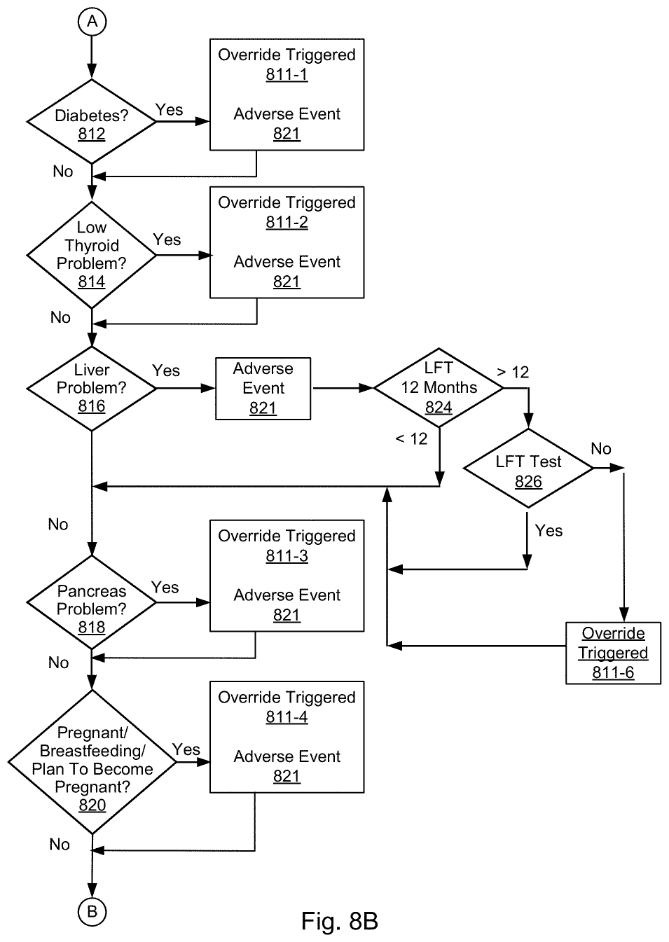

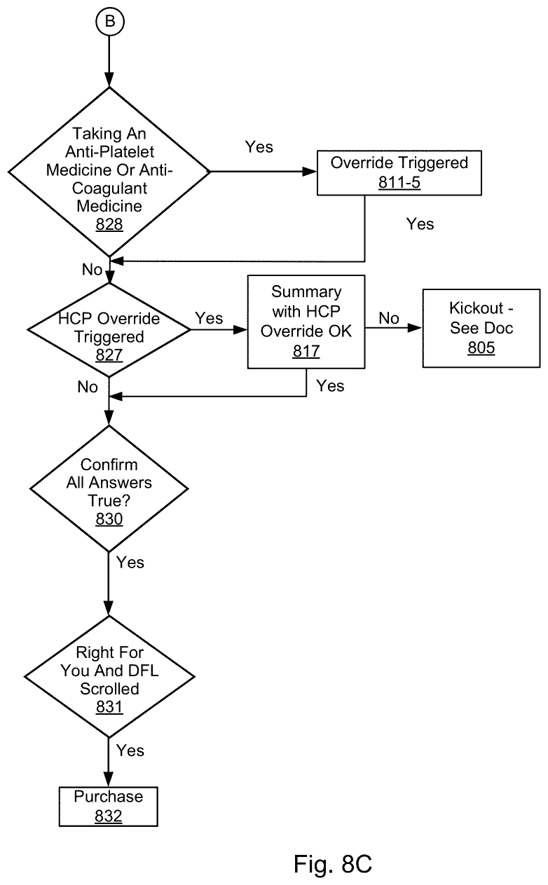

Referring to block 480 of FIG. 4F, the method also includes running all or a portion of the second survey results against a fourth plurality of filters of the second category class 220-2. When a respective filter in the fourth plurality of filters is fired, the subject is provided with a warning corresponding to the respective filter. In some embodiments, the warning is provided as a next step, e.g., prior to applying survey results to any subsequent filters, after the corresponding filter is fired. For example, with respect to FIGS. 8A-8C, in some embodiments, when the diabetes filter is triggered at 812, the device would provide the subject with a warning prior to proceeding to the thyroid disease filter at 814, e.g., requiring the subject confirm they have discussed their diabetes with a health care provider, e.g., and the healthcare provider still recommends taking a concentrated fish oil-based pharmaceutical composition. In some embodiments the warning is provided after applying survey results to all subsequent filters. For example, with respect to FIGS. 8A-8C, in some embodiments, when the diabetes filter is triggered at 812, the device would proceed to the thyroid disease filter at 814 prior to transmitting a warning to the subject, and then transmit all warnings corresponding to filters of the second category class, at 830, after survey results have been applied to all subsequent filters.

In some embodiments, the fourth plurality of filters of the second category class 220-2 includes some or all of the filters listed in Table 6. For example, in some embodiments, the fourth plurality of filters includes 2, 3, 4, 5, 6, or all 7 of the filters listed in Table 6. In some embodiments, the fourth plurality of filters of the second category class includes at least filters 1-6, as listed in Table 6.

TABLE 6
Example filters for risk factors associated with re-qualifying
a subject for an over-the-counter provision of a
metformin pharmaceutical composition

| Filter | Example Criteria |
|---|---|
| 1a | a diabetes filter |
| 2a | a thyroid disease filter |
| 3a | a liver disease filter |
| 4a | a pancreatic disease filter |
| 5a | a pregnancy filter |
| 6a | a coagulant medication filter |
| 7a | a side effects filter |

It is contemplated that, in some embodiments, any one or more of the filters provided in Table 6 will not be included in the fourth plurality of filters. For example, in some embodiments, a characteristic associated with a particular survey result will be informative when qualifying a subject for one particular concentrated fish oil but not for another concentrated fish oil. For instance, a survey question is queried for omega-3-carboxylic acid qualifying surveys but not for krill oil qualifying surveys. Accordingly, it is contemplated that the fourth plurality of filters includes any sub-set of filters provided in Table 6. Likewise, the skilled artisan may know of other filters, not provided in Table 6, that may be combined with any subset of the filters 222 provided in Table 6 to form the fourth plurality of filters results used in the methods described herein. For brevity, all possible combinations of the filters provided in Table 6 are not specifically delineated here.

Referring to block 482, in some embodiments, the fourth plurality of filters includes a diabetes filter. The diabetes filter is configured to be fired when the second plurality of survey results indicates that the subject has been diagnosed with diabetes since receiving their last provision of the concentrated fish oil-based pharmaceutical composition. In some embodiments, the second plurality of survey results indicates that the subject has developed diabetes when the second plurality of survey results include that the subject has been diagnosed with diabetes since receiving their last provision of the concentrated fish oil-based composition. When the diabetes filter is fired, the device transmits a warning corresponding to the diabetes filter, and requires the user to acknowledge the warning before authorizing a provision of the concentrated fish oil-based pharmaceutical composition.

Referring to block 482, in some embodiments, the fourth plurality of filters includes a thyroid disease filter that is configured to be fired at least when the second plurality of survey results indicates that the subject has been diagnosed with hypothyroidism since receiving their last provision of the concentrated fish oil-based pharmaceutical composition. In some embodiments, the second plurality of survey results indicates that the subject has developed hypothyroidism when the second plurality of survey results include that the subject has been diagnosed with hypothyroidism since receiving their last provision of the concentrated fish oil-based composition. When the thyroid disease filter is fired, the device transmits a warning corresponding to the thyroid disease filter, and requires the user to acknowledge the warning before authorizing a provision of the concentrated fish oil-based pharmaceutical composition

Referring to block 482, in some embodiments, the fourth plurality of filters includes a liver disease filter that is configured to be fired at least when the second survey results indicate that the subject has a liver disease. In some embodiments, the liver disease filter is configured to be fired when the second survey results indicate that the subject has developed symptoms of liver disease since receiving their last provision of the concentrated fish oil-based pharmaceutical composition. In some embodiments, the second plurality of survey results indicates that the subject has developed a liver disease when the second plurality of survey results include that the subject has been diagnosed with a liver disease since receiving their last provision of the concentrated fish oil-based composition. In some embodiments, the second plurality of survey results indicates that the subject has developed a liver disease when the second plurality of survey results include that the subject has experienced a symptom (e.g., a new and/or worsening symptom) of a liver disease since receiving their last provision of the concentrated fish oil-based composition, e.g., hepatic enzyme elevations (e.g., which are mostly consistent with cholestasis or hepatitis, jaundice, abdominal pain and/or swelling, swelling in the legs and/or ankles, irritated skin, a dark urine color, a pale, bloody, or tar-colored stool, chronic fatigue, nausea and/or vomiting, loss of appetite, and a tendency to bruise easily. When the liver disease filter is fired, the device transmits a warning corresponding to the liver disease filter, and requires the user to acknowledge the warning before authorizing a provision of the concentrated fish oil-based pharmaceutical composition.

Referring to block 482, in some embodiments, the fourth plurality of filters includes a pancreatic disease filter that is configured to be fired at least when the second plurality of survey results indicates that the subject has been diagnosed with pancreatic disease (e.g., pancreatitis) since receiving their last provision of the concentrated fish oil-based pharmaceutical composition. In some embodiments, the second plurality of survey results indicates that the subject has developed a pancreatic disease when the second plurality of survey results include that the subject has been diagnosed with a pancreatic disease since receiving their last provision of the concentrated fish oil-based composition. In some embodiments, the second plurality of survey results indicates that the subject has developed a pancreatic disease when the second plurality of survey results include that the subject has experienced a symptom (e.g., a new and/or worsening symptom) of a pancreatic disease since receiving their last provision of the concentrated fish oil-based composition. When the pancreatic disease filter is fired, the device transmits a warning corresponding to the pancreatic disease filter, and requires the user to acknowledge the warning before authorizing a provision of the concentrated fish oil-based pharmaceutical composition.

Referring to block 482, in some embodiments, the fourth plurality of filters includes a pregnancy filter (e.g., as described above in relation to the first survey). In some embodiments, the pregnancy filter is configured to be fired at least when the second plurality of survey results indicates that the subject is pregnant or the subject is breastfeeding. In some embodiments, the pregnancy filter is configured to be fired at least when the second plurality of survey results indicates that the subject is planning to become pregnant or planning to breastfeed. When the pregnancy filter is fired, the device transmits a warning corresponding to the pregnancy filter, and requires the user to acknowledge the warning before authorizing a provision of the concentrated fish oil-based pharmaceutical composition.

Referring to block 482, in some embodiments, the fourth plurality of filters includes a coagulant medication filter that is configured to be fired at least when the second plurality of survey results indicates that the subject is taking an anti-platelet medication or an anti-coagulant medication. When the coagulant medication filter is fired, the device transmits a warning corresponding to the coagulant filter, and requires the user to acknowledge the warning before authorizing a provision of the concentrated fish oil-based pharmaceutical composition.

Referring to block 484, in some embodiments the second survey results further include whether the subject has developed a side effect associated with the concentrated fish oil-based pharmaceutical composition since receiving their last provision of the concentrated fish oil-based pharmaceutical composition. Accordingly, in some embodiments, the fourth plurality of filters further includes a side effect filter that is configured to be fired at least when the second survey results indicate that the subject has developed a side effect since receiving their last provision of the concentrated fish oil-based pharmaceutical composition. Side effects that are capable of triggering the side effect filter include diarrhea, nausea, and abdominal discomfort. In some embodiments, side effects that are capable of triggering the side effect filter include to abdominal pain, abdominal distension, constipation, vomiting, fatigue, nasopharyngitis (e.g., a cold), dysgeusia (e.g., taste perversion), and/or eructation (e.g., belching). In some embodiments, side effects that are capable of triggering the side effect filter include arthralgia and/or oropharyngeal pain. In some embodiments, side effects that are capable of triggering the side effect filter include dyspepsia (e.g., indigestion), gastrointestinal disorder, an increase in alanine transaminase (ALT), an increase in aspartate aminotransferase (AST), pruritus, and/or skin rash. In some embodiments, side effects that are capable of triggering the side effect filter include a decreased appetite, heartburn, and/or flatulence. When the side effect filter is fired, the device transmits a warning corresponding to the side effect filter, and requires the user to acknowledge the warning before authorizing a provision of the concentrated fish-oil based pharmaceutical composition.

Referring to block 486, in some embodiments after triggering the liver disease filter, but prior to providing the subject with a warning corresponding to the triggering of the liver disease filter, a status of a previous liver function test taken by the subject is obtained. As previously described, in some embodiments information about the subject required for the methods described herein, e.g., one or more test results, is acquired from a preexisting database associated with the personal medical identification (e.g., a last liver function test or triglyceride level measurement determined for the subject). Similarly, in some embodiments information about the subject required for the methods described herein, e.g., one or more test results, is acquired directly from the subject.

Responsive to the obtained status of the previous liver function test taken by the subject a determination is made. When the status of the previous liver function test indicates the previous liver function test was taken less than a year ago, a determination is made to forgo providing the subject with the warning corresponding to the triggering of the liver disease filter. When the status of the previous liver function test indicates that the previous liver function test was taken more than a year ago, a status of the last liver function test taken by the subject is obtained in the second plurality of survey results.

Responsive to the obtained status of the last liver function test taken by the subject, a determination is made. When the status of the last liver function test indicates the last liver function test was taken less than a year ago, a determination is made to forgo providing the subject with the warning corresponding to triggering of the liver disease filter. When the status of the last liver function test indicates that the last liver function test was taken more than a year ago, the subject is provided with the warning corresponding to the triggering of the second liver disease filter.

In some embodiments, the second survey results further include whether the subject has developed an allergy to seafood and/or shellfish since receiving their last provision of the concentrated fish oil-based pharmaceutical composition. Accordingly, in some embodiments, the fourth plurality of filters further includes an allergy that is configured to be fired at least when the second survey results indicate that the subject has developed an allergy to seafood and/or shellfish since receiving their last provision of the concentrated fish oil-based pharmaceutical composition.

Referring to block 488 of FIG. 4G, in some embodiments when a respective filter in the third plurality of filters or fourth plurality of filters is fired, a record associated with the firing of the respective filter is stored (e.g., memorializing an adverse event that is required to be reported to a regulatory agency). This record is stored in an adverse event module 242 which comprises records of filter firing events associated with a plurality of subjects (e.g., an aggregation of adverse events associated with the concentrated fish oil-based pharmaceutical composition across a population of subjects taking the concentrated fish oil-based pharmaceutical composition over-the-counter). In some embodiments, an indication of the adverse event is communicated to a third party (e.g., a medical practitioner associated with the subject, a health care provider of the subject, a manufacturer/promoter of the concentrated fish oil-based pharmaceutical composition and/or a regulatory agency). In some embodiments, the indication is automatically stored in the adverse event module 242 when a response submitted by a subject as part of the second survey, triggers a filter associated with an adverse event.

Referring to block 490, in some embodiments the method also includes obtaining acknowledgment from the subject for each warning issued to the subject by any filter in the fourth plurality of filters. As described with respect to the warnings issued in conjunction with the second plurality of filters of the second category class, in some embodiments, the warning includes a prompt for the subject to indicate whether they have discussed the risk factor underlying the respective filter in the second plurality of filters that was fired with a health care practitioner (e.g., a licensed medical practitioner), e.g., and the health care practitioner indicated that the subject should take a concentrated fish oil-based pharmaceutical composition in view of the underlying risk factor. Accordingly, acknowledgement is obtained from the subject when the subject indicates that they have discussed the risk factor underlying the respective filter in the fourth plurality of filters that was fired with a health care provider.

Referring to block 492, in some embodiments the procedure further includes proceeding with the re-fulfillment process when the re-fulfillment process is not already terminated by the firing of a filter in the third plurality of filters (e.g., the second pregnancy filter). In order for completion of the re-fulfillment process, the subject is required to acknowledge each warning associated with each filter 222-2 in the fourth plurality of filters that was fired.

Referring to block 494, in some embodiments the re-fulfillment process also includes storing a record in the user profile 234 of the subject of a re-order 238 for the concentrated fish oil-based pharmaceutical composition.

The re-fulfillment process also includes communicating an over-the-counter drug facts label 230 for the concentrated fish oil-based pharmaceutical composition to the subject. As previously described, communication of the over-the-counter drug facts label 230 can occur in a variety of means. Upon confirmation from the subject that the over-the-counter drug facts label 230 has been received and read, the method includes authorizing a re-order provision of the concentrated fish oil-based pharmaceutical composition to the subject. In some embodiments, this re-order provision includes the destination of the subject.

FIG. 7 illustrates an example method (700) (e.g., performed at an electric device 102 or 250) for qualifying a subject for an over-the-counter concentrated fish oil-based pharmaceutical composition. In some embodiments, the method of FIG. 7 is utilized when the subject has not been previously qualified for the concentrated fish oil-based pharmaceutical composition. In some embodiments, the method of FIG. 7 is utilized when the subject was previously qualified for the concentrated fish oil-based pharmaceutical composition but a predetermined period of time elapsed since the previous qualification occurred (e.g., the most recent qualification/re-qualification of the subject was greater than one year ago).

Referring to FIG. 7, the device prompts (702) the subject to acknowledge a privacy notice. Since the present disclosure requires the subject to know and input sensitive medical information (e.g., information only the subject and a medical practitioner have access to), privacy of this information is important. Once the subject has acknowledged they have the requisite privacy for continuing, the device prompts (704) the user to confirm that they know their triglyceride level (e.g., because the subject must know their triglyceride level in order to complete the qualification process). If the subject indicates they do not know their triglyceride level, the process terminates (701) without authorizing provision of the concentrated fish oil-based pharmaceutical agent, and optionally transmits advice to the user to return later, e.g., once they know their triglyceride level. If the subject indicates they know their triglyceride level, the process continues.

The device prompts the subject to provide their triglyceride level and then applies (706,708) the answer received from the subject to a triglyceride level filter. When the triglyceride level filter is fired (e.g., when the answer indicates the subject has a triglyceride level that is less than 500 mg/dL or greater than 1000 mg/dL), the device terminates (705) the qualification process without authorizing provision of the concentrated fish oil-based pharmaceutical agent and, optionally, transmits advice for the subject to consult a medical professional.

When the triglyceride filter is not fired, the device proceeds with the qualification process, prompting the subject to provide their age and then applies (710) this information to an age filter. When the age filter is fired (e.g., when the subject is less than eighteen years old), the device terminates (703) the qualification process without authorizing provision of the concentrated fish oil-based pharmaceutical agent and, optionally, transmits advice to the user as to why they should not take the concentrated fish oil-based pharmaceutical agent.

When the age filter is not fired, the device proceeds with the qualification process, prompting the subject to provide a dietary status and then applies (712) the answer received from the subject to a dietary filter. When the dietary filter is fired (e.g., when the answer indicates the subject has a diet that is not low in saturated fat, low in cholesterol, low in carbohydrates, and/or low in added sugar), the device terminates (707) the qualification process without authorizing provision of the concentrated fish oil-based pharmaceutical agent and, optionally, transmits advice for the subject to maintain a healthy diet and to exercise.

When the diet filter is not fired, the device proceeds with the qualification process, prompting the subject to indicate their diabetes status and then applies (714) the answer received from the subject to a diabetes filter. When the diabetes filter is fired (e.g., when the answer indicates the subject has diabetes), the device initiates (711-1) an override procedure (e.g., creates a record indicating that the user must confirm they have discussed taking a concentrated fish oil-based pharmaceutical composition with a heath care provider). When the diabetes filter is not fired (e.g., when answer indicates the subject does not have diabetes), the device proceeds with the qualification process, prompting the subject to provide their thyroid status and then applies (716) the answer received from the subject to a thyroid disease filter. When the thyroid disease filter is fired (e.g., when the answer indicates the subject has thyroid disease), the device initiates (711-2) an override procedure (e.g., creates a record indicating that the user must confirm they have discussed taking a concentrated fish oil-based pharmaceutical composition with a heath care provider). When the thyroid disease filter is not fired (e.g., when answer indicates the subject does not have hypothyroidism), the device proceeds with the qualification process, prompting the subject to indicate whether the subject has a liver problem, and then applies (718) the answer received from the subject to a liver disease filter. When the liver disease filter is fired (e.g., when the answer indicates the subject has a liver problem), the device initiates (711-3) an override procedure (e.g., creates a record indicating that the user must confirm they have discussed taking a concentrated fish oil-based pharmaceutical composition with a heath care provider). When the liver disease filter is not fired (e.g., when answer indicates the subject does not have a liver problem), the device proceeds with the qualification process, prompting the subject to indicate whether the subject has a pancreatic problem, and then applies (720) the answer received from the subject to a pancreas disease filter. When the pancreas disease filter is fired (e.g., when the answer indicates the subject has a pancreatic problem), the device initiates (711-4) an override procedure (e.g., creates a record indicating that the user must confirm they have discussed taking a concentrated fish oil-based pharmaceutical composition with a heath care provider). When the pancreas disease filter is not fired (e.g., when answer indicates the subject does not have a pancreas problem), the device proceeds with the qualification process, prompting the subject to indicate whether the subject is one of pregnant, breastfeeding, or planning to become pregnant, and applies (722) the answer received from the subject to a pregnancy filter. When the pregnancy filter is fired (e.g., when the answer indicates the subject is one of pregnant, breastfeeding, or planning to become pregnant), the device initiates (711-5) an override procedure (e.g., creates a record indicating that the user must confirm they have discussed taking a concentrated fish oil-based pharmaceutical composition with a heath care provider). When the pregnancy filter is not fired (e.g., when the answer indicates the subject is not one of pregnant, breastfeeding, or planning to become pregnant), the device proceeds with the qualification process, prompting the subject to indicate whether the subject is allergic to seafood, and applies (723) the answer received from the subject to a seafood allergy filter. When the seafood allergy filter is fired (e.g., when the answer indicates the subject has an allergy to seafood), the device initiates (711-6) an override procedure (e.g., creates a record indicating that the user must confirm they have discussed taking a concentrated fish oil-based pharmaceutical composition with a heath care provider). When the seafood allergy filter is not fired (e.g., when the answer indicates the subject is not allergic to seafood), the device proceeds with the qualification process.

The device then proceeds with the qualification process, prompting the subject to provide information about their current anti-platelet or anti-coagulant medication use (e.g., whether the subject is currently taking an anti-platelet or an anti-coagulant medication) and then applies (724) the answer received from the subject to a coagulant medication filter. When coagulant medication filter is fired (e.g., when the answer indicates the subject is taking an anti-platelet or an anti-coagulant medication), the device initiates (711-7) an override procedure (e.g., creates a record indicating that the user must confirm they have discussed taking a concentrated fish oil-based pharmaceutical composition with a heath care provider).

The device proceeds with the qualification process, determining (725) whether the override procedure has been triggered (e.g., by firing of one or more of the diabetes, thyroid disease, liver disease, pancreas disease, pregnancy, seafood allergy and/or coagulant medication filters). If the override procedure has been triggered, the device prompts (717) the user to confirm that they have spoken with a medical professional about taking a concentrated fish oil-based pharmaceutical composition (e.g., in view of the underlying risk factor that triggered the diabetes, thyroid disease, liver disease, pancreas disease, pregnancy, seafood allergy and/or coagulant medication filters) and the medical professional recommended taking the concentrated fish oil-based pharmaceutical composition. If the user's response indicates they have not spoken with a medical professional or the medical professional did not recommend taking the concentrated fish oil-based pharmaceutical composition, the device terminates (703) the process and, optionally, transmits advice for the subject to consult a medical professional.

If the override procedure was not triggered, or the override procedure was triggered and the subject's response indicated that a medical professional recommended they take a concentrated fish oil-based pharmaceutical composition (e.g., in view of the underlying risk factor triggering the override procedure), the device then proceeds with the qualification process, prompting (726) the subject to confirm their answers. If the user confirms their answers, the device transmits (831) a drug facts label for the concentrated fish oil-based pharmaceutical composition and prompts the user to read the drug facts label. If the subject confirms they have read the drug facts label, the device proceeds to authorize (728) purchase of the concentrated fish oil-based pharmaceutical composition.

FIG. 8 illustrates an example method for qualifying a subject for a refill of an over-the-counter concentrated fish oil-based pharmaceutical composition (e.g., following a prescription from a medical professional or initial qualification by a method described herein). Referring to FIG. 8, the device prompts (802) the subject to acknowledge a privacy notice. Once the subject has acknowledged they have the requisite privacy for continuing, the device prompts (804) the user to confirm that they know their triglyceride level (e.g., because the subject must know their triglyceride level in order to complete the qualification process). In some embodiments, e.g., where the user has recently begun taking the concentrated fish oil-based pharmaceutical compound and/or the device has access to a recent a triglyceride level measurement from the subject, the device bypasses prompting the user to confirm that they know their triglyceride levels. If the subject indicates they do not know their triglyceride level, the device creates (821) a record of an adverse event (e.g., aggregated in an adverse event data store having records of adverse events from a plurality of users) and the process terminates (801) without authorizing provision of the concentrated fish oil-based pharmaceutical agent, and optionally transmits advice to the user to return later, e.g., once they know their triglyceride level. If the subject indicates they know their triglyceride level, the process continues, prompting the subject to indicate whether they are attempting to maintain an appropriate diet and applies (806) the answer received from the subject to a dietary filter. When the dietary filter is fired (e.g., when the answer indicates the subject is not attempting to maintain a diet which is low in saturated fats, low in cholesterol, low in carbohydrates, and/or low in added sugar), the device creates (821) a record of an adverse event (e.g., aggregated in an adverse event data store having records of adverse events from a plurality of users) and the process terminates (801) without authorizing provision of the concentrated fish oil-based pharmaceutical agent, and optionally transmits advice to the user to return later, e.g., once they have maintained an appropriate diet.

When the diet filter is not fired, the device proceeds with the qualification process, prompting the subject to provide information about their previous blood tests and then applies (808,810) the answer received from the subject to a triglyceride level filter. When the triglyceride level filter is fired (e.g., when the answer indicates the subject has not received a blood test in more than a year and has a triglyceride level that is greater than 500 mg/dL), the device creates (821) a record of an adverse event (e.g., aggregated in an adverse event data store having records of adverse events from a plurality of users), terminates (803) the qualification process and, optionally, transmits advice to the user as to why they should not take the concentrated fish oil-based pharmaceutical composition.

When the triglyceride level filter is not fired, the device proceeds with the qualification process, prompting the subject to indicate their diabetes status and then applies (812) the answer received from the subject to a diabetes filter. When the diabetes filter is fired (e.g. when the answer indicates that the subject has been diagnosed with diabetes since receiving their last provision of a concentrated fish oil-based pharmaceutical composition) the device initiates (811-1) an override procedure (e.g., creates a record indicating that the user must confirm they have discussed taking a concentrated fish oil-based pharmaceutical composition with a heath care provider). When the diabetes filter is not fired (e.g., when the answer indicates the subject has not been diagnosed with diabetes), the device proceeds with the qualification process, prompting the subject to provide their thyroid status and then applies (814) the answer received from the subject to a thyroid disease filter. When the thyroid disease filter is fired (e.g. when the answer indicates that the subject has been diagnosed with thyroid disease since receiving their last provision of a concentrated fish oil-based pharmaceutical composition) the device initiates (811-2) an override procedure (e.g., creates a record indicating that the user must confirm they have discussed taking a concentrated fish oil-based pharmaceutical composition with a heath care provider). When the thyroid disease filter is not fired (e.g., when answer indicates the subject has not been diagnosed with hypothyroidism), the device proceeds with the qualification process, prompting the subject to indicate a liver function status of the subject, and then applies (816) the answer received from the subject to a liver disease filter. When the liver disease filter is not fired (e.g., when answer indicates the subject has not been diagnosed with a liver problem), the device proceeds with the qualification process, prompting the subject to indicate whether the subject has a pancreatic problem, and then applies (818) the answer received from the subject to a pancreas disease filter. When the pancreas disease filter is fired (e.g. when the answer indicates that the subject has been diagnosed with pancreatic disease since receiving their last provision of a concentrated fish oil-based pharmaceutical composition) the device initiates (811-3) an override procedure (e.g., creates a record indicating that the user must confirm they have discussed taking a concentrated fish oil-based pharmaceutical composition with a heath care provider). When the pancreas disease filter is not fired (e.g., when answer indicates the subject has not been diagnosed with a pancreas problem), the device proceeds with the qualification process, prompting the subject to indicate whether the subject is one of pregnant, breastfeeding, or planning to become pregnant, and applies (820) the answer received from the subject to a pregnancy filter. When the pregnancy filter is fired (e.g. when the answer indicates that the subject is pregnant, breastfeeding, or planning to become pregnant since receiving their last provision of a concentrated fish oil-based pharmaceutical composition) the device initiates (811-4) an override procedure (e.g., creates a record indicating that the user must confirm they have discussed taking a concentrated fish oil-based pharmaceutical composition with a heath care provider). When the pregnancy filter is not fired (e.g., when the answer indicates the subject has not become one of pregnant, breastfeeding, or planning to become pregnant) the device proceeds with the qualification process. When any one of the diabetes filter, the thyroid disease filter, the liver disease filter, the pancreas disease filter, and/or the pregnancy filter are fired (e.g., when the answers indicates the subject has been diagnosed with diabetes, has been diagnosed with hypothyroidism, has been diagnosed with a liver problem, has been diagnosed with a pancreas problem, and/or has become one of pregnant, breastfeeding, or planning to become pregnant), after initiating one or more override procedures (811), the device creates (821) a record of an adverse event (e.g., aggregated in an adverse event data store having records of adverse events from a plurality of users). Upon creation of the record of the adverse event, the device determines (822) if the liver filter was fired and obtains a status of a last liver function test of the subject. The device applies (824) the obtained status of the last liver function test of the subject to the liver disease filter in order to determine (826) if the subject needs to take another liver function test. In some embodiments, after the user confirms they have spoken with a medical practitioner, the device prompts the user to confirm that they have actually discussed the underlying condition with a medical practitioner.

The device then proceeds with the qualification process, prompting the subject to provide information about their current coagulant medication use and then applies (828) the answer received from the subject to a coagulant medication filter. When the coagulant medication filter is fired (e.g., when the answer indicates the subject has started taking an anti-platelet or an anti-coagulant medication since receiving their last provision of a concentrated fish oil-based pharmaceutical composition), the device initiates (811-4) an override procedure (e.g., creates a record indicating that the user must confirm they have discussed taking a concentrated fish oil-based pharmaceutical composition with a heath care provider).

The device proceeds with the qualification process, determining (827) whether the override procedure has been triggered (e.g., by firing of one or more of the diabetes, thyroid disease, liver disease, pancreas disease, pregnancy, and/or coagulant medication filters). If the override procedure has been triggered, the device prompts (817) the user to confirm that they have spoken with a medical professional about taking a concentrated fish oil-based pharmaceutical composition (e.g., in view of the underlying risk factor that triggered the diabetes, thyroid disease, liver disease, pancreas disease, pregnancy, and/or coagulant medication filters) and the medical professional recommended taking the concentrated fish oil-based pharmaceutical composition. If the user's response indicates they have not spoken with a medical professional or the medical professional did not recommend taking the concentrated fish oil-based pharmaceutical composition, the device terminates (805) the process and, optionally, transmits advice for the subject to consult a medical professional.

If the override procedure was not triggered, or the override procedure was triggered and the subject's response indicated that a medical professional recommended they take a concentrated fish oil-based pharmaceutical composition (e.g., in view of the underlying risk factor triggering the override procedure), the device then proceeds with the qualification process, prompting (830) the subject to confirm their answers. If the user confirms their answers, the device transmits (831) a drug facts label for the concentrated fish oil-based pharmaceutical composition and prompts the user to read the drug facts label. If the subject confirms they have read the drug facts label, the device proceeds to authorize (832) purchase of the concentrated fish oil-based pharmaceutical composition.

### Specific Embodiments

In one aspect, the disclosure provides methods, software, and computer systems for qualifying a human subject for over-the-counter delivery of a concentrated fish oil-based pharmaceutical composition for lowering triglyceride levels, e.g., thereby, treating or preventing hypertriglyceridemia and/or heart disease. In one embodiment, a computer system (e.g., computer system 250 in FIG. 2) includes instructions for conducting a survey of the subject (e.g., survey module 204 in FIG. 2) to obtain information about the subject necessary to run against at least two series of filters (e.g., first filter category class 214 in FIG. 2 and second filter category class 220 in FIG. 2). The computer system also includes instructions for running the survey results against the filters. Filters 216 in the first series of filters prevent authorization for delivery of the OTC concentrated fish oil where the subject's survey results identify a contraindication for the OTC concentrated fish oil. Filters 222 in the second series of filters generate a warning 226 where the subject's survey results identify a risk factor for the OTC concentrated fish oil. In some embodiments, the warning 226 includes a prompt requiring the subject to confirm they have discussed the risk factor with a physician in order to proceed with qualification for the OTC concentrated fish oil.

In one aspect, the disclosure provides methods, software, and computer systems for qualifying a human subject for a re-order for over-the-counter delivery of a concentrated fish oil-based pharmaceutical composition for lowering triglyceride levels, e.g., thereby treating or preventing hypertriglyceridemia and/or heart disease. In one embodiment, a computer system includes instructions, responsive to receiving a re-order request from the subject for the concentrated fish oil-based pharmaceutical composition, performing a re-fulfillment procedure comprising, for conducting a second survey of the subject to obtain survey results for qualifying the subject for the re-order, e.g., associated with at least two series of filters (e.g., a third series of filters of a first category class 214-2 in FIG. 2 and fourth series of filters of a second category class 220-2 in FIG. 2). The computer system also includes instructions for running the survey results against the filters. Filters 216 in the third series of filters prevent authorization for delivery of the OTC concentrated fish oil where the subject's survey results identify a contraindication for the OTC concentrated fish oil. Filters 222 in the fourth series of filters generate a warning 226 where the subject's survey results identify a risk factor for the OTC concentrated fish oil. In some embodiments, the warning 226 includes a prompt requiring the subject to confirm they have discussed the risk factor with a physician in order to proceed with qualification for the OTC concentrated fish oil.

The computer system includes instructions for proceeding with a re-fulfillment process only when no filters in the third series of filters was fired and the subject acknowledged each warning associated with each filter in the fourth plurality of filters that was fired. The computer system also includes instructions for storing an indication in a subject profile of a re-order for the concentrated fish oil-based pharmaceutical composition The computer system also includes instructions for communicating an over-the-counter drug facts label 230 for the concentrated fish oil-based pharmaceutical composition to the subject and, upon confirmation that the over-the-counter drug facts label has been received and read, authorizing provision of the OTC concentrated fish oil-based pharmaceutical composition to the subject.

In one aspect, the disclosure provides a computer system for qualifying a human subject for over-the-counter delivery of a concentrated fish oil-based pharmaceutical composition for lowering triglyceride levels, e.g., thereby treating or preventing hypertriglyceridemia and/or heart disease. The computer system comprising one or more processors and a memory, the memory comprising non-transitory instructions which, when executed by the one or more processor, perform a method for qualifying a human subject for over-the-counter delivery of the concentrated fish oil-based pharmaceutical composition. The method includes conducting a first survey of the subject thereby obtaining a first plurality of survey results necessary to run against a first plurality of filters of a first category class and a second plurality of filters of a second category class. The method also includes running all or a portion of the first plurality of survey results against a first plurality of filters of a first category class, wherein, when a respective filter in the first plurality of filters is fired, the subject is deemed not qualified for delivery of the concentrated fish oil-based pharmaceutical composition and the method is terminated without delivery of the concentrated fish oil-based pharmaceutical composition to the subject. The method also includes running all or a portion of the first plurality of survey results against a second plurality of filters of a second category class, wherein, when a respective filter in the second plurality of filters is fired, the subject is provided with a warning corresponding to the respective filter. The method also includes obtaining acknowledgment from the subject for the warning issued to the subject by any filter in the second plurality of filters. The method also includes proceeding with a fulfillment process when no filter in the first plurality of filters has been fired and the subject has acknowledged each warning associated with each filter in the second plurality of filters that was fired. The fulfillment process includes: storing an indication in a subject profile of an initial order for the concentrated fish oil-based pharmaceutical composition, communicating an over-the-counter drug facts label for the concentrated fish oil-based pharmaceutical composition to the subject, and authorizing, upon confirmation from the subject that the over-the-counter drug facts label has been received and read, provision of the concentrated fish oil-based pharmaceutical composition to the subject. In some embodiments, the authorization includes a destination associated with the subject.

In some embodiments, the first plurality of survey results includes a plurality of survey results selected from the survey results listed in Table 1. In one embodiment, the first plurality of survey results includes: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation.

In some embodiments, the first plurality of filters includes a plurality of filters selected from the filters listed in Table 2. In one embodiment, the first plurality of filters includes a triglyceride level filter, an age filter, a dietary filter, and an allergy filter.

In some embodiments, the second plurality of filters includes a plurality of filters selected from the filters listed in Table 3. In one embodiment, the second plurality of filters includes a diabetes filter, a thyroid disease filter, a liver disease filter, a pancreatic disease filter, a pregnancy filter, an allergy filter, and a coagulant medication filter.

In some embodiments, the first and second plurality of filters includes filters selected from the filters listed in Table 8. In some embodiments, the first plurality of filters of the first category class include a first sub-plurality of the filters listed in Table 8, for example, 2, 3, 4, 5, 6, 7, 8, 9, 10, or all 11 of the filters listed in Table 8, and the second plurality of filters of the first category class include a second sub-plurality of the filters listed in Table 8, which is different from the first sub-plurality of filters, for example, 2, 3, 4, 5, 6, 7, 8, 9, 10, or all 11 of the filters listed in Table 8. In some embodiments, each of the filters in the first sub-plurality of filters is different from each of the filters in the second sub-plurality of filters (e.g., no filter listed in Table 8 is included in both the first sub-plurality and the second sub-plurality of filters). In some embodiments, a system for qualifying a subject for delivery of an over-the-counter concentrated fish oil-based pharmaceutical composition includes instructions for applying only one plurality of filters, e.g., only filters of a single category class of filters. In some embodiments, where the method, system, or software applies a single plurality of filters, the plurality of filters includes a plurality of filters selected from the filters listed in Table 8, e.g., at least 2, 3, 4, 5, 6, 7, 8, 9, 10, or all 11 of the filters listed in Table 8. In some embodiments, where a filter listed in Table 8 corresponds to a filter listed in Table 2 or Table 3, a threshold level sufficient to fire the corresponding filter listed in Table 2 or Table 3, as described in detail above, is sufficient to fire the filter listed in Table 8.

TABLE 8
Example filters for qualifying a subject for an over-the-counter provision
of a concentrated fish oil-based pharmaceutical composition

| Filter | Example Criteria |
|---|---|
| 1b | a triglyceride level filter |
| 2b | an age filter |
| 3b | a dietary filter |
| 4b | a fish oil allergy filter |
| 5b | a diabetes filter |
| 6b | a thyroid disease filter |
| 7b | a liver disease filter |
| 8b | a pancreatic disease filter |
| 9b | a pregnancy filter |
| 10b | a seafood allergy filter |
| 11b | a coagulant medication filter |

In one aspect, the disclosure provides methods, software, and computer systems for qualifying a human subject for a re-order of an over-the-counter provision of a concentrated fish oil-based pharmaceutical composition for lowering triglycerides, e.g., thereby treating or preventing heart disease. In one embodiment, a computer system includes instructions, responsive to receiving a re-order request from the subject for the concentrated fish oil-based pharmaceutical composition, for performing a re-fulfillment procedure comprising conducting a second survey of the subject, thereby obtaining a second plurality of survey results necessary to run against a third plurality of filters of a first category class and a fourth plurality of filters of a second category class. The method also includes running all or a portion of the second plurality of survey results against a third plurality of filters of a first category class, wherein, when a respective filter in the third plurality of filters is fired, the subject is deemed not qualified for delivery of the concentrated fish oil-based pharmaceutical composition and the method is terminated without delivery of the concentrated fish oil-based pharmaceutical composition to the subject. The method also includes running all or a portion of the second plurality of survey results against a fourth plurality of filters of a second category class, wherein, when a respective filter in the fourth plurality of filters is fired, the subject is provided with a warning corresponding to the respective filter. The method also includes obtaining acknowledgment from the subject for the warning issued to the subject by any filter in the fourth plurality of filters. The method also includes proceeding with a re-fulfillment process when no filter in the third plurality of filters has been fired and the subject has acknowledged each warning associated with each filter in the fourth plurality of filters that was fired. The re-fulfillment process includes: storing an indication in a subject profile of a re-order for the concentrated fish oil-based pharmaceutical composition, communicating the over-the-counter drug facts label for the concentrated fish oil-based pharmaceutical composition to the subject, and authorizing, upon confirmation from the subject that the over-the-counter drug facts label has been received and read, provision of the concentrated fish oil-based pharmaceutical composition to the subject.

In some embodiments, the third series of filters includes one or more filters listed in Table 5. In some embodiments, the third plurality of filters includes a dietary filter and a triglyceride filter.

In some embodiments, the fourth series of filters includes one or more filters listed in Table 6. In some embodiments, the fourth plurality of filters includes a diabetes filter, a thyroid disease filter, a liver disease filter, a pancreatic disease filter, a pregnancy filter, a coagulant medication filter, and a coagulant medication filter.

In some embodiments, the third and fourth plurality of filters includes filters selected from the filters listed in Table 9. In some embodiments, the third plurality of filters of the first category class include a third sub-plurality of the filters listed in Table 9, for example, 2, 3, 4, 5, or all 6 of the filters listed in Table 9, and the fourth plurality of filters of the first category class include a fourth sub-plurality of the filters listed in Table 9, which is different from the third sub-plurality of filters, for example, 2, 3, 4, 5, or all 6 of the filters listed in Table 9. In some embodiments, each of the filters in the third sub-plurality of filters is different from each of the filters in the fourth sub-plurality of filters (e.g., no filter listed in Table 9 is included in both the first sub-plurality and the second sub-plurality of filters). In some embodiments, a system for qualifying a subject for delivery of an over-the-counter concentrated fish oil-based pharmaceutical composition includes instructions for applying only one plurality of filters, e.g., only filters of a single category class of filters. In some embodiments, where the method, system, or software applies a single plurality of filters, the plurality of filters includes a plurality of filters selected from the filters listed in Table 9, e.g., at least 2, 3, 4, 5, or all 6 of the filters listed in Table 9. In some embodiments, where a filter listed in Table 9 corresponds to a filter listed in Table 2, Table 3, Table 5, or Table 6, a threshold level sufficient to fire the corresponding filter listed in Table 2, Table 3, Table 5, or Table 6, as described in detail above, is sufficient to fire the filter listed in Table 9.

TABLE 9
Example filters for re-qualifying a subject for an
over-the-counter provision of a concentrated
fish-oil based pharmaceutical composition

| Filter | Example Criteria |
|---|---|
| 1b | a dietary filter |
| 2b | a triglyceride level filter |
| 3b | a diabetes filter |
| 4b | a thyroid disease filter |
| 5b | a liver disease filter |
| 6b | a pancreatic disease filter |
| 7b | a pregnancy filter |
| 8b | a coagulant medication filter |
| 9b | a side effects filter |

In one aspect, the present disclosure provides a computer system for qualifying a human subject for over-the-counter delivery of a concentrated fish oil-based pharmaceutical composition for lowering triglyceride levels, e.g., thereby treating or preventing hypertriglyceridemia and/or heart disease, the computer system comprising one or more processors and a memory, the memory comprising non-transitory instructions which, when executed by the one or more processor, perform a method comprising: a) conducting a first survey of the subject thereby obtaining a first plurality of survey results, wherein the first plurality of survey results indicates: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation; b) running all or a portion of the first plurality of survey results against a first plurality of filters of a first category class, wherein, when a respective filter in the first plurality of filters is fired, the subject is deemed not qualified for delivery of the concentrated fish oil-based pharmaceutical composition and the method is terminated without delivery of the concentrated fish oil-based pharmaceutical composition to the subject, wherein the first plurality of filters comprises: a first triglyceride level filter that is fired at least when the first plurality of survey results indicates that the subject has a triglyceride level that is either below a floor triglyceride level or above a ceiling triglyceride level, an age filter, and a first dietary filter that is fired at least when the first plurality of survey results indicates that the subject's diet is not appropriate; c) running all or a portion of the first plurality of survey results against a second plurality of filters of a second category class, wherein, when a respective filter in the second plurality of filters is fired, the subject is provided with a warning corresponding to the respective filter, and wherein the second plurality of filters comprises: a first diabetes filter that is fired at least when the first plurality of survey results indicates that the subject has diabetes, a first thyroid disease filter that is fired at least when the first plurality of survey results indicates that the subject has hypothyroidism, a first liver disease filter that is fired at least when the first plurality of survey results indicates that the subject has a liver problem, a first pancreatic disease filter that is fired at least when the first plurality of survey results indicates that the subject has a pancreatic problem, a first pregnancy filter that is fired at least when the first plurality of survey results indicates that the subject is pregnant or the subject is breastfeeding, a first allergy filter that is fired at least when the first plurality of survey results indicates that the subject is allergic to fish or shellfish, and a first coagulant medication filter that is fired at least when the first plurality of survey results indicates that the subject is taking an anti-platelet medication or an anti-coagulant medication; d) obtaining acknowledgment from the subject for the warning issued to the subject by any filter in the second plurality of filters; and e) proceeding with a fulfillment process when (i) no filter in the first plurality of filters has been fired and (ii) the subject has acknowledged each warning associated with each filter in the second plurality of filters that was fired, wherein the fulfillment process comprises: storing an indication in a subject profile of an initial order for the concentrated fish oil-based pharmaceutical composition, communicating an over-the-counter drug facts label for the fish oil-based pharmaceutical composition to the subject, and authorizing, upon confirmation from the subject that the over-the-counter drug facts label has been received and read, provision of the fish oil-based pharmaceutical composition to the subject.

In some embodiments of the aspects disclosed above, the concentrated fish oil-based pharmaceutical composition includes an omega-3-carboxylic acid pharmaceutical composition.

In some embodiments of the aspects disclosed above, upon confirmation from the subject that the over the counter drug facts label has been received and read, the subject is authorized for provision of a dosage of from 0.5 g to 2.5 g of an omega-3-carboxylic acid pharmaceutical composition no more than once per day

In some embodiments of the aspects disclosed above, upon confirmation from the subject that the over the counter drug facts label has been received and read, the subject is authorized for provision of a dosage of 2 g of an omega-3-carboxylic acid pharmaceutical composition no more than once per day

In some embodiments of the aspects disclosed above, the concentrated fish oil-based pharmaceutical composition includes an omega-3 acid ethyl ester pharmaceutical composition, an ethyl eicosapentaenoic acid pharmaceutical composition, or a krill oil pharmaceutical composition.

In some embodiments of the aspects disclosed above, the floor triglyceride level used in the first triglyceride level filter is 500 mg/dL.

In some embodiments of the aspects disclosed above, the age filter is fired when the first plurality of survey results indicates that the subject is less than eighteen years old.

In some embodiments of the aspects disclosed above, the first diet filter is fired when the first plurality of survey results indicates that the subject's diet is not low in at least one of saturated fat, cholesterol, carbohydrates, and added sugar.

In some embodiments of the aspects disclosed above the first pregnancy filter is also fired at least when the first plurality of survey results indicates that the subject plans to become pregnant.

In some embodiments of the aspects disclosed above, the first plurality of survey results further comprises whether the subject is allergic to the concentrated fish oil-based pharmaceutical composition, and the first plurality of filters includes an adverse reaction filter that is fired when the first plurality of survey results indicates that the subject is allergic to the concentrated fish oil-based pharmaceutical composition.

In some embodiments of the aspects disclosed above, the warning corresponding to a respective filter in the second plurality of filters comprises a prompt for the subject to indicate whether they have discussed the risk factor underlying the respective filter in the second plurality of filters that was fired with a health care provider; and acknowledgement is obtained from the subject when the subject indicates that they have discussed the risk factor underlying the respective filter in the second plurality of filters that was fired with a health care provider.

In some embodiments of the aspects disclosed above, the fulfillment process further comprises: storing a destination associated with the subject in the subject profile.

In some embodiments of the aspects disclosed above, the fulfillment process further comprises: coordinating shipping of the concentrated fish oil-based pharmaceutical composition to a physical address associated with the subject.

In some embodiments of the aspects disclosed above, the method further comprises: f) responsive to receiving a re-order request from the subject for the concentrated fish oil-based pharmaceutical composition, performing a re-fulfillment procedure comprising: (i) conducting a second survey of the subject thereby obtaining a second plurality of survey results, wherein the second plurality of survey results indicate: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation; (ii) running all or a portion of the second plurality of survey results against a third plurality of filters of the first category class, wherein, when a respective filter in the third plurality of filters is fired, the subject is deemed not qualified for the concentrated fish oil-based pharmaceutical composition and the re-fulfillment process is terminated without delivery of the concentrated fish oil-based pharmaceutical composition to the subject, wherein the third plurality of filters comprise: a second dietary filter that is fired at least when the second plurality of survey results indicates that the subject is not attempting to maintain an appropriate diet, and a second triglyceride level filter that is fired at least when the second plurality of survey results indicates that the subject is not maintaining an appropriate triglyceride level; (iii) running all or a portion of the second plurality of survey results against a fourth plurality of filters of the second category class, wherein, when a respective filter in the fourth plurality of filters is fired, the subject is provided with a warning corresponding to the respective filter, and wherein the fourth plurality of filters comprises: a second diabetes filter that is fired at least when the second plurality of survey results indicates that the subject has been diagnosed with diabetes since receiving their last provision of the concentrated fish oil-based pharmaceutical composition, a second thyroid disease filter that is fired at least when the second plurality of survey results indicates that the subject has been diagnosed with hypothyroidism since receiving their last provision of the concentrated fish oil-based pharmaceutical composition, a second liver disease filter that is fired at least when the second plurality of survey results indicates that the subject has liver disease, a second pancreatic disease filter that is fired at least when the second plurality of survey results indicates that the subject has been diagnosed with pancreatic disease since receiving their last provision of the concentrated fish oil-based pharmaceutical composition, a second pregnancy filter that is fired at least when the second plurality of survey results indicates that the subject is pregnant or the subject is breastfeeding, and a second coagulant medication filter that is fired at least when the second plurality of survey results indicates that the subject is taking an anti-platelet medication or an anti-coagulant medication; (iv) obtaining acknowledgment from the subject for the warning issued to the subject by any filter in the fourth plurality of filters; (v) proceeding with the re-fulfillment process when (a) the re-fulfillment process is not already terminated by the firing of a filter in the third plurality of filters and (b) the subject has acknowledged each warning associated with each filter in the fourth plurality of filters that was fired, wherein the re-fulfillment process further comprises: storing an indication in the subject profile of a re-order for the concentrated fish oil-based pharmaceutical composition, communicating the over-the-counter drug facts label for the concentrated fish oil-based pharmaceutical composition to the subject, and authorizing, upon confirmation from the subject that the over-the-counter drug facts label has been received and read, a re-order provision of the concentrated fish oil-based pharmaceutical composition to the subject.

In some embodiments of the aspects disclosed above, the second dietary filter is fired when the second plurality of survey results indicates that the subject is not attempting to maintain a diet that is low in saturated fat, low in cholesterol, low in carbohydrates, and low in added sugar.

In some embodiments of the aspects disclosed above, the second triglyceride level filter is fired when a predetermined period of time has passed since the user took the last blood test indicated in the subject profile for the subject, and the second plurality of survey results indicates that the subject has a triglyceride level that is above a maintenance triglyceride level.

In some embodiments of the aspects disclosed above, the maintenance triglyceride level used in the second triglyceride level filter is 500 mg/dL.

In some embodiments of the aspects disclosed above, after triggering the second liver disease filter and prior to providing the subject with a warning corresponding to the triggering of the second liver disease filter, a status of a previous liver function test taken by the subject is obtained. Responsive to this: when the status of the previous liver function test indicates the previous liver function test was taken less than a year ago, forgo providing the subject with the warning corresponding to the triggering of the second liver disease filter, and when the status of the previous liver function test indicates that the previous liver function test was taken less than a year ago, obtaining, in the second plurality of survey results, a status of the last liver function test taken by the subject. Moreover, responsive to obtaining the status of the last liver function test taken by the subject: when the status of the last liver function test indicates the last liver function test was taken less than a year ago, forgo providing the subject with the warning corresponding to triggering of the second liver disease filter, and when the status of the last liver function test indicates that the last liver function test was taken less than a year ago, proceeding to provide the subject with the warning corresponding to the triggering of the second liver disease filter.

In some embodiments of the aspects disclosed above, the second plurality of survey results further comprises whether the subject has developed a side effect associated with the concentrated fish oil-based pharmaceutical composition since receiving their last provision of the concentrated fish oil-based pharmaceutical composition, and the fourth plurality of filters further comprises a side effect filter that is fired at least when the second plurality of survey results indicates that the subject has developed, since receiving their last provision of the concentrated fish oil-based pharmaceutical composition, a side effect selected from the group consisting of diarrhea, nausea, and abdominal discomfort.

In one aspect, the disclosure provides a method for lowering triglycerides in a subject in need thereof, the method comprising: administering a (e.g., low-dose) concentrated fish oil-based pharmaceutical composition to a subject qualified for over-the-counter access to the concentrated fish oil-based pharmaceutical composition. In some embodiments, the subject is qualified for the over-the-counter access to the concentrated fish oil-based pharmaceutical composition using a method, system, or computer readable medium disclosed herein.

In some embodiments, the concentrated fish oil-based pharmaceutical composition includes an omega-3-carboxylic acid pharmaceutical composition.

In some embodiments of the aspects disclosed above, upon confirmation from the subject that the over the counter drug facts label has been received and read, the subject is authorized for provision of a dosage of from 0.5 g to 2.5 g of an omega-3-carboxylic acid pharmaceutical composition no more than once per day

In some embodiments of the aspects disclosed above, upon confirmation from the subject that the over the counter drug facts label has been received and read, the subject is authorized for provision of a dosage of 2 g of an omega-3-carboxylic acid pharmaceutical composition no more than once per day.

In some embodiments of the aspects disclosed above, the concentrated fish oil-based pharmaceutical composition includes an omega-3 acid ethyl ester pharmaceutical composition, an ethyl eicosapentaenoic acid pharmaceutical composition, or a krill oil pharmaceutical composition.

In some embodiments of the aspects disclosed above, the concentrated fish oil is ethyl eicosapentaenoic acid and the subject is administered a dosage of from 1 g to 4 g per day. In some embodiments, the dosage of ethyl eicosapentaenoic acid is from 2 g to 4 g per day. In some embodiments, the dosage of ethyl eicosapentaenoic acid is 4 g per day.

In some embodiments of the aspects disclosed above, the concentrated fish oil is krill oil and the subject is administered a dosage of from 1 g to 4 g per day. In some embodiments, the dosage of krill oil is from 2 g to 4 g per day. In some embodiments, the dosage of krill oil is 4 g per day.

In some embodiments, the disclosure provides methods for lowering triglycerides with an over the counter concentrated fish oil-based pharmaceutical composition. The method includes providing a first survey for obtaining a first information set from the human, via a computer system having a processor programed to perform the first survey, where the first information set includes information about the human that relates to potential risk factors and contraindications for the concentrated fish oil-based pharmaceutical composition, as described herein. The method also includes applying an algorithm to the first information set, via a computer system having a processor programed to perform the algorithm. The algorithm runs all or a portion of the first information set against a first plurality of filters, where the human is deemed not qualified for treatment with the over the counter concentrated fish oil-based pharmaceutical composition for lowering triglycerides when a respective filter in the first plurality of filters is fired and the method is terminated without authorizing provision of the concentrated fish oil-based pharmaceutical composition to the human, where the first plurality of filters includes filters related to contraindications of the concentrated fish oil-based pharmaceutical composition as described herein. The algorithm also runs all or a portion of the first information set against a second plurality of filters, where, when a respective filter in the second plurality of filters is fired, the human is provided with a warning corresponding to the respective filter, and where the second plurality of filters includes filters related to risk factors for the concentrated fish oil-based pharmaceutical composition as described herein. The algorithm also obtains acknowledgment from the human of the risk factor associated with each warning issued to the human by any filter in the second plurality of filters. In some embodiments, the acknowledgement includes confirmation that the human has discussed the risk factor with a physician. The algorithm proceeds with a fulfillment process when (a) no filter in the first plurality of filters has been fired and (b) the human has acknowledged each warning associated with each filter in the second plurality of filters that was fired. The fulfillment process includes storing an indication in a subject profile of an initial order for the concentrated fish oil-based pharmaceutical composition, communicating an over the counter drug facts label for the concentrated fish oil-based pharmaceutical composition to the human, and authorizing, upon confirmation from the subject that the over the counter drug facts label has been received and read, provision of the concentrated fish oil-based pharmaceutical composition to the human, where the authorization includes a destination associated with the subject. In some embodiments, the method also includes treating the human to lower the triglycerides of the human, upon authorization of the provision e.g., by providing access to the concentrated fish oil-based pharmaceutical composition to the human and/or by administering the concentrated fish oil-based pharmaceutical composition to lower triglycerides in the human.

### EXAMPLES

### Example 1

A computer system is configured for qualifying a subject for over-the-counter delivery of an omega-3-carboxylic acid pharmaceutical composition (e.g., (4Z, 7Z, 10Z, 13Z, 16Z, 19Z)-docosa-4,7,10,13,16,19-hexaenoic acid;(4Z, 7Z, 10Z, 13Z, 16Z)-docosa-4,7,10,13,16-pentaenoic acid;(7Z, 10Z, 13Z, 16Z, 19Z)-docosa-7,10,13,16,19-pentaenoic acid;(6Z, 9Z, 12Z, 15Z, 18Z)-henicosa-6,9,12,15,18-pentaenoic acid;(5Z, 8Z, 11Z, 14Z, 17Z)-icosa-5,8,11,14,17-pentaenoic acid;(8Z, 11Z, 14Z, 17Z)-icosa-8,11,14,17-tetraenoic acid;(9Z, 12Z)-octadeca-9,12-dienoic acid;(6Z, 9Z, 12Z)-octadeca-6,9,12-trienoic acid;(9Z, 12Z, 15Z)-octadeca-9,12,15-trienoic acid) for lowering triglyceride levels, e.g., thereby, treating or preventing hypertriglyceridemia and/or heart disease. The computer system includes instructions for conducting a survey of the subject. The survey is utilized to obtain one or more results of: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation.

The computer system runs survey results against a first series of filters that are each associated with a first filter category class. The first filter category class is configured to prevent authorization for OTC delivery of the OTC omega-3-carboxylic acid when the subject's survey results identify a contraindication for the omega-3-carboxylic acid. In some embodiments, the first series of filters includes one or more of a first triglyceride level filter, an age filter, and a first dietary filter. The triglyceride level filter is configured to ensure the subject has a high triglyceride level. The age filter is configured to ensure the subject is greater than or equal to eighteen years old. Furthermore, the dietary filter is configured to ensure the subject has a diet that is low in saturated fat, low in cholesterol, low in carbohydrates, and/or low in added sugar.

The computer system runs survey results against a second series of filters that each generates a warning where the subject's survey results identify a risk factor for the OTC omega-3-carboxylic acid. In some embodiments, the second series of filters includes a first diabetes filter, a first thyroid filter, a first liver disease filter, a first pancreatic disease filter, a first pregnancy filter, a fire allergy filter, and a first coagulant medication filter. The first diabetes filter is configured to ensure that the subject does not have diabetes. The first thyroid disease filter is configured to ensure that the subject does not have hypothyroidism. The first liver disease filter is configured to ensure that the subject does not have a liver problem. The first pancreatic disease filter is configured to ensure that the subject does not have a pancreatic problem. The first pregnancy filter is configured to ensure that the subject is not pregnant or the subject is not breastfeeding. The first allergy filter is configured to ensure that the subject is not allergic to fish or shellfish. Furthermore, the first coagulant medication filter is configured to ensure that the subject is not taking an anti-platelet medication or an anti-coagulant medication.

The computer system then prompts the subject to acknowledge or deny having discussed these warnings with a medical professional (e.g., their physician or healthcare provider). The computer system then proceeds with a fulfillment process only when none of the first series of filters was fired and the subject acknowledged that they discussed each warning issued in association with the second series of filters that was fired.

The computer system stores an indication of an initial order of the OTC omega-3-carboxylic acid in a subject profile, and communicates an over-the-counter drug facts label for the omega-3-carboxylic acid pharmaceutical composition to the subject. Upon confirmation from the subject that they have received and read the over-the-counter drug facts label, the computer system authorizes provision of the OTC omega-3-carboxylic acid pharmaceutical composition to the subject.

In some embodiments, the computer system includes instructions for conducting another survey of the subject responsive to a re-order request of the omega-3-carboxylic acid pharmaceutical composition. This survey is utilized to obtain one or more results of: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation.

The computer system runs survey results against a third series of filters that are each associated with the first filter category class. In some embodiments, the third series of filters includes one or more of a second dietary filter and a second triglyceride level filter. This dietary filter is fired at least when the second plurality of survey results indicates that the subject is not attempting to maintain an appropriate diet. This triglyceride level filter is fired at least when the second plurality of survey results indicates that the subject is not maintaining an appropriate triglyceride level.

The computer system runs survey results against a fourth series of filters that each generates a warning where the subject's survey results identify a risk factor for the OTC omega-3-carboxylic acid. In some embodiments, the fourth series of filters includes a second diabetes filter, a second thyroid disease filter, a second liver disease filter, a second pancreatic disease filter, a second pregnancy filter, and a second coagulant medication filter. The second diabetes filter is fired at least when the second plurality of survey results indicates that the subject has been diagnosed with diabetes since receiving their last provision of the omega-3-carboxylic acid pharmaceutical composition. The second thyroid disease filter is fired at least when the second plurality of survey results indicates that the subject has been diagnosed with hypothyroidism since receiving their last provision of the omega-3-carboxylic acid pharmaceutical composition. The second liver disease filter is fired at least when the second plurality of survey results indicates that the subject has liver disease. The second pancreatic disease filter is fired at least when the second plurality of survey results indicates that the subject has been diagnosed with pancreatic disease since receiving their last provision of the omega-3-carboxylic acid pharmaceutical composition. The second pregnancy filter is fired at least when the second plurality of survey results indicates that the subject is pregnant or the subject is breastfeeding. The second coagulant medication filter is fired at least when the second plurality of survey results indicates that the subject is taking an anti-platelet medication or an anti-coagulant medication.

The computer system then prompts the subject to acknowledge or deny having discussed these warnings with a medical professional (e.g., their physician or healthcare provider). The computer system then proceeds with a re-fulfillment process only when none of the third series of filters was fired the subject acknowledged that they discussed each warning issued in association with the fourth series of filters that was fired.

The computer system stores an indication of a re-order of the OTC omega-3-carboxylic acid in the subject profile, and communicates the over-the-counter drug facts label for the omega-3-carboxylic acid pharmaceutical composition to the subject. Upon confirmation from the subject that they have received and read the over-the-counter drug facts label, the computer system authorizes provision of the OTC omega-3-carboxylic acid pharmaceutical composition to the subject.

### Example 2

A computer system is configured for qualifying a subject for over-the-counter delivery of an omega-3 acid ethyl ester pharmaceutical composition (e.g., ethyl (4Z, 7Z, 10Z, 13Z, 16Z, 19Z)-docosa-4,7,10,13,16,19-hexaenoate;ethyl (5Z, 8Z, 11Z, 14Z, 17Z)-icosa-5,8,11,14,17-pentaenoate) for lowering triglyceride levels, e.g., thereby, treating or preventing hypertriglyceridemia and/or heart disease. The computer system includes instructions for conducting a survey of the subject. The survey is utilized to obtain one or more results of: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation.

The computer system runs survey results against a first series of filters that are each associated with a first filter category class. The first filter category class is configured to prevent authorization for OTC delivery of the OTC omega-3 acid ethyl ester when the subject's survey results identify a contraindication for the omega-3 acid ethyl ester. In some embodiments, the first series of filters includes one or more of a first triglyceride level filter, an age filter, and a first dietary filter. The triglyceride level filter is configured to ensure the subject has a high triglyceride level. The age filter is configured to ensure the subject is greater than or equal to eighteen years old. Furthermore, the dietary filter is configured to ensure the subject has a diet that is low in saturated fat, low in cholesterol, low in carbohydrates, and/or low in added sugar.

The computer system runs survey results against a second series of filters that each generates a warning where the subject's survey results identify a risk factor for the OTC omega-3 acid ethyl ester. In some embodiments, the second series of filters includes a first diabetes filter, a first thyroid filter, a first liver disease filter, a first pancreatic disease filter, a first pregnancy filter, a fire allergy filter, and a first coagulant medication filter. The first diabetes filter is configured to ensure that the subject does not have diabetes. The first thyroid disease filter is configured to ensure that the subject does not have hypothyroidism. The first liver disease filter is configured to ensure that the subject does not have a liver problem. The first pancreatic disease filter is configured to ensure that the subject does not have a pancreatic problem. The first pregnancy filter is configured to ensure that the subject is not pregnant or the subject is not breastfeeding. The first allergy filter is configured to ensure that the subject is not allergic to fish or shellfish. Furthermore, the first coagulant medication filter is configured to ensure that the subject is not taking an anti-platelet medication or an anti-coagulant medication.

The computer system then prompts the subject to acknowledge or deny having discussed these warnings with a medical professional (e.g., their physician or healthcare provider). The computer system then proceeds with a fulfillment process only when none of the first series of filters was fired and the subject acknowledged that they discussed each warning issued in association with the second series of filters that was fired.

The computer system stores an indication of an initial order of the OTC omega-3 acid ethyl ester in a subject profile, and communicates an over-the-counter drug facts label for the omega-3 acid ethyl ester pharmaceutical composition to the subject. Upon confirmation from the subject that they have received and read the over-the-counter drug facts label, the computer system authorizes provision of the OTC omega-3 acid ethyl ester pharmaceutical composition to the subject.

In some embodiments, the computer system includes instructions for conducting another survey of the subject responsive to a re-order request of the omega-3 acid ethyl ester pharmaceutical composition. This survey is utilized to obtain one or more results of: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation.

The computer system runs survey results against a third series of filters that are each associated with the first filter category class. In some embodiments, the third series of filters includes one or more of a second dietary filter and a second triglyceride level filter. This dietary filter is fired at least when the second plurality of survey results indicates that the subject is not attempting to maintain an appropriate diet. This triglyceride level filter is fired at least when the second plurality of survey results indicates that the subject is not maintaining an appropriate triglyceride level.

The computer system runs survey results against a fourth series of filters that each generates a warning where the subject's survey results identify a risk factor for the OTC omega-3 acid ethyl ester. In some embodiments, the fourth series of filters includes a second diabetes filter, a second thyroid disease filter, a second liver disease filter, a second pancreatic disease filter, a second pregnancy filter, and a second coagulant medication filter. The second diabetes filter is fired at least when the second plurality of survey results indicates that the subject has been diagnosed with diabetes since receiving their last provision of the omega-3 acid ethyl ester pharmaceutical composition. The second thyroid disease filter is fired at least when the second plurality of survey results indicates that the subject has been diagnosed with hypothyroidism since receiving their last provision of the omega-3 acid ethyl ester pharmaceutical composition. The second liver disease filter is fired at least when the second plurality of survey results indicates that the subject has liver disease. The second pancreatic disease filter is fired at least when the second plurality of survey results indicates that the subject has been diagnosed with pancreatic disease since receiving their last provision of the omega-3 acid ethyl ester pharmaceutical composition. The second pregnancy filter is fired at least when the second plurality of survey results indicates that the subject is pregnant or the subject is breastfeeding. The second coagulant medication filter is fired at least when the second plurality of survey results indicates that the subject is taking an anti-platelet medication or an anti-coagulant medication.

The computer system then prompts the subject to acknowledge or deny having discussed these warnings with a medical professional (e.g., their physician or healthcare provider). The computer system then proceeds with a re-fulfillment process only when none of the third series of filters was fired the subject acknowledged that they discussed each warning issued in association with the fourth series of filters that was fired.

The computer system stores an indication of a re-order of the OTC omega-3 acid ethyl ester in the subject profile, and communicates the over-the-counter drug facts label for the omega-3 acid ethyl ester pharmaceutical composition to the subject. Upon confirmation from the subject that they have received and read the over-the-counter drug facts label, the computer system authorizes provision of the OTC omega-3 acid ethyl ester pharmaceutical composition to the subject.

### Example 3

A computer system is configured for qualifying a subject for over-the-counter delivery of an ethyl eicosapentaenoic acid pharmaceutical composition Ethyl (5Z, 8Z, 11Z, 14Z, 17Z)-eicosa-5,8,11,14,17-pentaenoate for lowering triglyceride levels, e.g., thereby treating or preventing hypertriglyceridemia and/or heart disease. The computer system includes instructions for conducting a survey of the subject. The survey is utilized to obtain one or more results of: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation.

The computer system runs survey results against a first series of filters that are each associated with a first filter category class. The first filter category class is configured to prevent authorization for OTC delivery of the OTC ethyl eicosapentaenoic acid when the subject's survey results identify a contraindication for the ethyl eicosapentaenoic acid. In some embodiments, the first series of filters includes one or more of a first triglyceride level filter, an age filter, and a first dietary filter. The triglyceride level filter is configured to ensure the subject has a high triglyceride level. The age filter is configured to ensure the subject is greater than or equal to eighteen years old. Furthermore, the dietary filter is configured to ensure the subject has a diet that is low in saturated fat, low in cholesterol, low in carbohydrates, and/or low in added sugar.

The computer system runs survey results against a second series of filters that each generates a warning where the subject's survey results identify a risk factor for the OTC ethyl eicosapentaenoic acid. In some embodiments, the second series of filters includes a first diabetes filter, a first thyroid filter, a first liver disease filter, a first pancreatic disease filter, a first pregnancy filter, a fire allergy filter, and a first coagulant medication filter. The first diabetes filter is configured to ensure that the subject does not have diabetes. The first thyroid disease filter is configured to ensure that the subject does not have hypothyroidism. The first liver disease filter is configured to ensure that the subject does not have a liver problem. The first pancreatic disease filter is configured to ensure that the subject does not have a pancreatic problem. The first pregnancy filter is configured to ensure that the subject is not pregnant or the subject is not breastfeeding. The first allergy filter is configured to ensure that the subject is not allergic to fish or shellfish. Furthermore, the first coagulant medication filter is configured to ensure that the subject is not taking an anti-platelet medication or an anti-coagulant medication.

The computer system then prompts the subject to acknowledge or deny having discussed these warnings with a medical professional (e.g., their physician or healthcare provider). The computer system then proceeds with a fulfillment process only when none of the first series of filters was fired and the subject acknowledged that they discussed each warning issued in association with the second series of filters that was fired.

The computer system stores an indication of an initial order of the OTC ethyl eicosapentaenoic acid in a subject profile, and communicates an over-the-counter drug facts label for the ethyl eicosapentaenoic acid pharmaceutical composition to the subject. Upon confirmation from the subject that they have received and read the over-the-counter drug facts label, the computer system authorizes provision of the OTC ethyl eicosapentaenoic acid pharmaceutical composition to the subject.

In some embodiments, the computer system includes instructions for conducting another survey of the subject responsive to a re-order request of the ethyl eicosapentaenoic acid pharmaceutical composition. This survey is utilized to obtain one or more results of: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation.

The computer system runs survey results against a third series of filters that are each associated with the first filter category class. In some embodiments, the third series of filters includes one or more of a second dietary filter and a second triglyceride level filter. This dietary filter is fired at least when the second plurality of survey results indicates that the subject is not attempting to maintain an appropriate diet. This triglyceride level filter is fired at least when the second plurality of survey results indicates that the subject is not maintaining an appropriate triglyceride level.

The computer system runs survey results against a fourth series of filters that each generates a warning where the subject's survey results identify a risk factor for the OTC ethyl eicosapentaenoic acid. In some embodiments, the fourth series of filters includes a second diabetes filter, a second thyroid disease filter, a second liver disease filter, a second pancreatic disease filter, a second pregnancy filter, and a second coagulant medication filter. The second diabetes filter is fired at least when the second plurality of survey results indicates that the subject has been diagnosed with diabetes since receiving their last provision of the ethyl eicosapentaenoic acid pharmaceutical composition. The second thyroid disease filter is fired at least when the second plurality of survey results indicates that the subject has been diagnosed with hypothyroidism since receiving their last provision of the ethyl eicosapentaenoic acid pharmaceutical composition. The second liver disease filter is fired at least when the second plurality of survey results indicates that the subject has liver disease. The second pancreatic disease filter is fired at least when the second plurality of survey results indicates that the subject has been diagnosed with pancreatic disease since receiving their last provision of the ethyl eicosapentaenoic acid pharmaceutical composition. The second pregnancy filter is fired at least when the second plurality of survey results indicates that the subject is pregnant or the subject is breastfeeding. The second coagulant medication filter is fired at least when the second plurality of survey results indicates that the subject is taking an anti-platelet medication or an anti-coagulant medication.

The computer system then prompts the subject to acknowledge or deny having discussed these warnings with a medical professional (e.g., their physician or healthcare provider). The computer system then proceeds with a re-fulfillment process only when none of the third series of filters was fired the subject acknowledged that they discussed each warning issued in association with the fourth series of filters that was fired.

The computer system stores an indication of a re-order of the OTC ethyl eicosapentaenoic acid in the subject profile, and communicates the over-the-counter drug facts label for the ethyl eicosapentaenoic acid pharmaceutical composition to the subject. Upon confirmation from the subject that they have received and read the over-the-counter drug facts label, the computer system authorizes provision of the OTC ethyl eicosapentaenoic acid pharmaceutical composition to the subject.

### Example 4

A computer system is configured for qualifying a subject for over-the-counter delivery of a krill oil pharmaceutical composition for lowering triglyceride levels, e.g., thereby treating or preventing hypertriglyceridemia and/or heart disease. The computer system includes instructions for conducting a survey of the subject. The survey is utilized to obtain one or more results of: a triglyceride level of the subject, an age of the subject, a dietary status of the subject, a diabetes status of the subject, a thyroid status of the subject, whether the subject has a liver problem, whether the subject has a pancreatic problem, whether the subject is one of pregnant, breastfeeding, or planning to become pregnant, whether the subject has a seafood allergy, and whether the subject is taking a medication the affects coagulation.

The computer system runs survey results against a first series of filters that are each associated with a first filter category class. The first filter category class is configured to prevent authorization for OTC delivery of the OTC krill oil when the subject's survey results identify a contraindication for the krill oil. In some embodiments, the first series of filters includes one or more of a first triglyceride level filter, an age filter, and a first dietary filter. The triglyceride level filter is configured to ensure the subject has a high triglyceride level. The age filter is configured to ensure the subject is greater than or equal to eighteen years old. Furthermore, the dietary filter is configured to ensure the subject has a diet that is low in saturated fat, low in cholesterol, low in carbohydrates, and/or low in added sugar.

The computer system runs survey results against a second series of filters that each generates a warning where the subject's survey results identify a risk factor for the OTC krill oil. In some embodiments, the second series of filters includes a first diabetes filter, a first thyroid filter, a first liver disease filter, a first pancreatic disease filter, a first pregnancy filter, a fire allergy filter, and a first coagulant medication filter. The first diabetes filter is configured to ensure that the subject does not have diabetes. The first thyroid disease filter is configured to ensure that the subject does not have hypothyroidism. The first liver disease filter is configured to ensure that the subject does not have a liver problem. The first pancreatic disease filter is configured to ensure that the subject does not have a pancreatic problem. The first pregnancy filter is configured to ensure that the subject is not pregnant or the subject is not breastfeeding. The first allergy filter is configured to ensure that the subject is not allergic to fish or shellfish. Furthermore, the first coagulant medication filter is configured to ensure that the subject is not taking an anti-platelet medication or an anti-coagulant medication.

The computer system then prompts the subject to acknowledge or deny having discussed these warnings with a medical professional (e.g., their physician or healthcare provider). The computer system then proceeds with a fulfillment process only when none of the first series of filters was fired and the subject acknowledged that they discussed each warning issued in association with the second series of filters that was fired.

The computer system stores an indication of an initial order of the OTC krill oil in a subject profile, and communicates an over-the-counter drug facts label for the krill oil pharmaceutical composition to the subject. Upon confirmation from the subject that they have received and read the over-the-counter drug facts label, the computer system authorizes provision of the OTC krill oil pharmaceutical composition to the subject.

In some embodiments, the computer system includes instructions for conducting another survey of the subject responsive to a re-order request of the krill oil pharmaceutical composition. This survey is utilized to obtain one or more results of: a dietary status of the subject, if a predetermined period of time has passed since the user took a previous blood test, a triglyceride level status of the subject, a diabetes status of the subject, a thyroid status of the subject, a liver function status of the subject, a pancreatic function status of the subject, whether the subject is one of pregnant, breastfeeding, or planning to become pregnant, and whether the subject is taking a medication that affects coagulation.

The computer system runs survey results against a third series of filters that are each associated with the first filter category class. In some embodiments, the third series of filters includes one or more of a second dietary filter and a second triglyceride level filter. This dietary filter is fired at least when the second plurality of survey results indicates that the subject is not attempting to maintain an appropriate diet. This triglyceride level filter is fired at least when the second plurality of survey results indicates that the subject is not maintaining an appropriate triglyceride level.

The computer system runs survey results against a fourth series of filters that each generates a warning where the subject's survey results identify a risk factor for the OTC krill oil. In some embodiments, the fourth series of filters includes a second diabetes filter, a second thyroid disease filter, a second liver disease filter, a second pancreatic disease filter, a second pregnancy filter, and a second coagulant medication filter. The second diabetes filter is fired at least when the second plurality of survey results indicates that the subject has been diagnosed with diabetes since receiving their last provision of the krill oil pharmaceutical composition. The second thyroid disease filter is fired at least when the second plurality of survey results indicates that the subject has been diagnosed with hypothyroidism since receiving their last provision of the krill oil pharmaceutical composition. The second liver disease filter is fired at least when the second plurality of survey results indicates that the subject has liver disease. The second pancreatic disease filter is fired at least when the second plurality of survey results indicates that the subject has been diagnosed with pancreatic disease since receiving their last provision of the krill oil pharmaceutical composition. The second pregnancy filter is fired at least when the second plurality of survey results indicates that the subject is pregnant or the subject is breastfeeding. The second coagulant medication filter is fired at least when the second plurality of survey results indicates that the subject is taking an anti-platelet medication or an anti-coagulant medication.

The computer system then prompts the subject to acknowledge or deny having discussed these warnings with a medical professional (e.g., their physician or healthcare provider). The computer system then proceeds with a re-fulfillment process only when none of the third series of filters was fired the subject acknowledged that they discussed each warning issued in association with the fourth series of filters that was fired.

The computer system stores an indication of a re-order of the OTC krill oil in the subject profile, and communicates the over-the-counter drug facts label for the krill oil pharmaceutical composition to the subject. Upon confirmation from the subject that they have received and read the over-the-counter drug facts label, the computer system authorizes provision of the OTC krill oil pharmaceutical composition to the subject.

### References Cited and Alternative Embodiments

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 1, 2, and 3 and/or described in FIG. 4 or 5. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A computer system for qualifying a human subject for over-the-counter delivery of a concentrated fish oil-based pharmaceutical composition for lowering triglycerides, the computer system comprising one or more processors and a memory, the memory comprising non-transitory instructions which, when executed by the one or more processor, perform a method comprising:
a) conducting a first survey of the subject thereby obtaining a first plurality of survey results, wherein the first plurality of survey results comprises:
a triglyceride level of the subject,
an age of the subject,
a dietary status of the subject,
a diabetes status of the subject,
a thyroid status of the subject,
whether the subject has a liver problem,
whether the subject has a pancreatic problem,
whether the subject is pregnant or breastfeeding,
whether the subject has a seafood allergy, and
whether the subject is taking a medication the affects coagulation;
b) running all or a portion of the first plurality of survey results against a first plurality of filters of a first category class, wherein, when a respective filter in the first plurality of filters is fired, the subject is deemed not qualified for delivery of the concentrated fish oil-based pharmaceutical composition and the method is terminated without delivery of the concentrated fish oil-based pharmaceutical composition to the subject, wherein the first plurality of filters comprises:
a first triglyceride level filter that is fired at least when the first plurality of survey results indicates that the subject has a triglyceride level that is either (i) below a baseline triglyceride level, or (ii) above a ceiling triglyceride level,
an age filter, and
a first dietary filter that is fired at least when the first plurality of survey results indicates that the subject's diet is not appropriate;
c) running all or a portion of the first plurality of survey results against a second plurality of filters of a second category class, wherein, when a respective filter in the second plurality of filters is fired, the subject is provided with a warning corresponding to the respective filter, and wherein the second plurality of filters comprises:
a first diabetes filter that is fired at least when the first plurality of survey results indicates that the subject has diabetes,
a first thyroid disease filter that is fired at least when the first plurality of survey results indicates that the subject has hypothyroidism,
a first liver disease filter that is fired at least when the first plurality of survey results indicates that the subject has a liver problem,
a first pancreatic disease filter that is fired at least when the first plurality of survey results indicates that the subject has a pancreatic problem,
a first pregnancy filter that is fired at least when the first plurality of survey results indicates that the subject is pregnant or the subject is breastfeeding,
a first allergy filter that is fired at least when the first plurality of survey results indicates that the subject is allergic to fish or shellfish, and
a first coagulant medication filter that is fired at least when the first plurality of survey results indicates that the subject is taking an anti-platelet medication or an anti-coagulant medication;
d) obtaining acknowledgment from the subject for the warning issued to the subject by any filter in the second plurality of filters; and
e) proceeding with a fulfillment process when (i) no filter in the first plurality of filters has been fired and (ii) the subject has acknowledged each warning associated with each filter in the second plurality of filters that was fired, wherein the fulfillment process comprises:
storing an indication in a subject profile of an initial order for the concentrated fish oil-based pharmaceutical composition,
communicating an over the counter drug facts label for the concentrated fish oil-based pharmaceutical composition to the subject, and
authorizing, upon confirmation from the subject that the over the counter drug facts label has been received and read, provision of the concentrated fish oil-based pharmaceutical composition to the subject.

2. The computer system of claim 1, wherein the concentrated fish oil-based pharmaceutical composition is an omega-3-carboxylic acid pharmaceutical composition.

3. The computer system of claim 2, wherein the over the counter drug facts label specifies that the omega-3-carboxylic acid pharmaceutical composition is to be taken by the subject at a dosage of 0.5 g to 2 g per day.

4. The computer system of claim 2, wherein the over the counter drug facts label specifies that the omega-3-carboxylic acid pharmaceutical composition is to be taken by the subject at a dosage of 2 g per day.

5. The computer system of claim 1, wherein the concentrated fish oil-based pharmaceutical composition is an omega-3 acid ethyl ester pharmaceutical composition, an ethyl eicosapentaenoic acid pharmaceutical composition, or a krill oil pharmaceutical composition.

6. The computer system of claim 1, wherein the baseline triglyceride level used in the first triglyceride level filter is 500 mg/dL.

7. The computer system of claim 1, wherein the ceiling triglyceride level is used in the first triglyceride level filter is 1000 mg/dL.

8. The computer system of claim 1, wherein the age filter is fired when the first plurality of survey results indicates that the subject is less than eighteen years old.

9. The computer system of claim 1, wherein the first diet filter is fired when the first plurality of survey results indicates that the subject's diet is not low in at least one of saturated fat, cholesterol, carbohydrates, and added sugar.

10. (canceled)

11. The computer system of claim 1, wherein:
the first plurality of survey results further comprises whether the subject is allergic to the concentrated fish oil-based pharmaceutical composition, and
the first plurality of filters includes a second allergy filter that is fired when the first plurality of survey results indicates that the subject is allergic to the concentrated fish oil-based pharmaceutical composition.

12. The computer system of claim 1, wherein:
the warning corresponding to a respective filter in the second plurality of filters comprises a prompt for the user to indicate whether they have discussed the risk factor underlying the respective filter in the second plurality of filters that was fired with a health care provider; and
acknowledgement is obtained from the subject when the subject indicates that they have discussed the risk factor underlying the respective filter in the second plurality of filters that was fired with a health care provider.

13. The computer system of claim 1, wherein the fulfillment process further comprises:
storing a destination associated with the subject in the subject profile.

14. The computer system of claim 1, wherein the fulfillment process further comprises:
coordinating shipping of the concentrated fish oil-based pharmaceutical composition to a physical address associated with the subject.

15. The computer system of claim 1, wherein the method further comprises:
f) responsive to receiving a re-order request from the subject for the concentrated fish oil-based pharmaceutical composition, performing a re-fulfillment procedure comprising:
(i) conducting a second survey of the subject thereby obtaining a second plurality of survey results, wherein the second plurality of survey results comprises:
a dietary status of the subject,
if a threshold amount of time has passed since the user took a previous blood test, a triglyceride level status of the subject,
a diabetes status of the subject,
a thyroid status of the subject,
a liver function status of the subject,
a pancreatic function status of the subject,
whether the subject is pregnant or breastfeeding, and
whether the subject is taking a medication that affects coagulation;
(ii) running all or a portion of the second plurality of survey results against a third plurality of filters of the first category class, wherein, when a respective filter in the third plurality of filters is fired, the subject is deemed not qualified for the concentrated fish oil-based pharmaceutical composition and the re-fulfillment process is terminated without delivery of the concentrated fish oil-based pharmaceutical composition to the subject, wherein the third plurality of filters comprises:
a second dietary filter that is fired at least when the second plurality of survey results indicates that the subject is not attempting to maintain an appropriate diet, and
a second triglyceride level filter that is fired at least when the second plurality of survey results indicates that the subject is not maintaining an appropriate triglyceride level;
(iii) running all or a portion of the second plurality of survey results against a fourth plurality of filters of the second category class, wherein, when a respective filter in the fourth plurality of filters is fired, the subject is provided with a warning corresponding to the respective filter, and wherein the fourth plurality of filters comprises:
a second diabetes filter that is fired at least when the second plurality of survey results indicates that the subject has been diagnosed with diabetes since receiving their last provision of the concentrated fish oil-based pharmaceutical composition,
a second thyroid disease filter that is fired at least when the second plurality of survey results indicates that the subject has been diagnosed with hypothyroidism since receiving their last provision of the concentrated fish oil-based pharmaceutical composition,
a second liver disease filter that is fired at least when the second plurality of survey results indicates that the subject has liver disease,
a second pancreatic disease filter that is fired at least when the second plurality of survey results indicates that the subject has been diagnosed with pancreatic disease since receiving their last provision of the concentrated fish oil-based pharmaceutical composition,
a second pregnancy filter that is fired at least when the second plurality of survey results indicates that the subject is pregnant or the subject is breastfeeding, and
a second coagulant medication filter that is fired at least when the second plurality of survey results indicates that the subject is taking an anti-platelet medication or an anti-coagulant medication;
(iv) obtaining acknowledgment from the subject for the warning issued to the subject by any filter in the fourth plurality of filters; and
(v) proceeding with the re-fulfillment process when (a) the re-fulfillment process is not already terminated by the firing of a filter in the third plurality of filters and (b) the subject has acknowledged each warning associated with each filter in the fourth plurality of filters that was fired, wherein the re-fulfillment process further comprises:
storing an indication in a subject profile of a re-order for the concentrated fish oil-based pharmaceutical composition,
communicating the over the counter drug facts label for the concentrated fish oil-based pharmaceutical composition to the subject, and
authorizing, upon confirmation from the subject that the over the counter drug facts label has been received and read, a re-order provision of the concentrated fish oil-based pharmaceutical composition to the subject.

16. The computer system according of claim 15, wherein the second dietary filter is fired when the second plurality of survey results indicates that the subject is not attempting to maintain a diet that is low in saturated fat, low in cholesterol, low in carbohydrates, and low in added sugar.

17. The computer system of claim 15, wherein the second triglyceride level filter is fired when:
(A) a predetermined period of time has passed since the user took the last blood test indicated in the subject profile for the subject, and
(B) the second plurality of survey results indicates that the subject has a triglyceride level that is above a maintenance triglyceride level.

18. The computer system of claim 17, wherein the maintenance triglyceride level used in the second triglyceride level filter is 500 mg/dL.

19. The computer system of claim 15, further comprising, after triggering the second liver disease filter and prior to providing the subject with a warning corresponding to the triggering of the second liver disease filter:
obtaining a status of a previous liver function test taken by the subject; and
responsive to obtaining the status of the previous liver function test taken by the subject:
when the status of the previous liver function test indicates the previous liver function test was taken less than a year ago, forgo providing the subject with the warning corresponding to the triggering of the second liver disease filter, and
when the status of the previous liver function test indicates that the previous liver function test was taken less than a year ago, obtaining, in the second plurality of survey results, a status of the last liver function test taken by the subject and, responsive to obtaining the status of the last liver function test taken by the subject:
when the status of the last liver function test indicates the last liver function test was taken less than a year ago, forgo providing the subject with the warning corresponding to triggering of the second liver disease filter, and
when the status of the last liver function test indicates that the last liver function test was taken less than a year ago, proceeding to provide the subject with the warning corresponding to the triggering of the second liver disease filter.

20. The computer system of claim 15, wherein:
the second plurality of survey results further comprises whether the subject has experienced a side effect from the concentrated fish oil-based pharmaceutical composition, and
the fourth plurality of filters further comprises a side effect filter that is fired at least when the second plurality of survey results indicates that the subject has experienced, since receiving their last provision of the concentrated fish oil-based pharmaceutical composition, a side effect selected from the group consisting of diarrhea, nausea, and abdominal discomfort.

21. A method for lowering triglycerides in a subject in need thereof, the method comprising:
administering a concentrated fish oil-based pharmaceutical composition to a subject qualified for over-the-counter access to the concentrated fish oil-based blocker pharmaceutical composition.

22-28. (canceled)
